(19) United States
(12) Patent Application Publication
(10) Pub. No.: US 2022/0305250 A1
(43) Pub. Date: Sep. 29, 2022

(54) Cannula System Comprising Two Cannulas and Corresponding Method

(71) Applicant: ReCO2very Therapies GmbH, Bad Klosterlausnitz (DE)

(72) Inventor: Torsten HEILMANN, Bad Klosterlausnitz (DE)

(21) Appl. No.: 17/635,771

(22) PCT Filed: Aug. 19, 2020
(86) PCT No.: PCT/EP2020/073249

§ 371 Date: Feb. 16, 2022

(30) Foreign Application Priority Data
Aug. 19, 2019 (EP) PCT/EP2019/072164

(51) Int. Cl. A61M 60/13; A61M 60/148; A61M 60/139; A61M 60/237; A61M 60/32; A61M 60/414; A61M 60/569

(52) U.S. Cl. CPC A61M 60/13; A61M 60/148; A61M 60/139; A61M 60/237; A61M 60/32; A61M 60/414; A61M 60/569

(57) ABSTRACT

Disclosed is a Cannula system (CS1 to CS3, 210, 310, 1010, 1040, 1110) comprising: a first cannula (O1 to O3) having a minimum inner diameter or width, a second cannula (I1 to I3) having a maximum outer diameter or width, wherein the minimum inner diameter or width is greater than the maximum outer diameter or width, wherein the second cannula (I1 to I3) is adapted to be guided into the first cannula (O1 to O3) such that a first lumen of the first cannula (O1 to O3) remains in the first cannula (O1 to O3) between an outer surface the second cannula (I1 to I3) and an inner surface of the first cannula (O1 to O3), and wherein the first lumen of the first cannula (O1 to O3) defines a first fluid conduit and a first lumen of the second cannula (I1 to I3) defines a second fluid conduit.

The invention relates to a cannula system comprising at least two cannulas, for instance an outer cannula or a first cannula and an inner cannula or second cannula. Dual lumen cannulas are known for a long time. Usually both lumen are guided into the body of a subject at the same time.

The basic principle of an endovascular catheter/cannula therapy may be a treatment of vessels and/or by using vessels for the advancement of a catheter, for instance plastic tubes or plastic tubes that are armed with metal. An incision may be made into the skin of a patient. The incision may have a length that is less than 5 cm (centimeter), less than 3 cm or less than 1 cm. Local anesthesia may be used thereby. A cannula may be used to insert a guide wire and/or dilators to expand the incision and/or an opening within the vessel. The catheter may be inserted using the guide wire and/or an introducing member.

No thoracotomy may be necessary if cannulas or catheters are used. A cannula may be a tube that can be inserted into the body, often for the delivery or removal of fluid or for the gathering of data. A catheter may be a thin tube made from medical grade materials serving a broad range of functions. Catheters may be medical devices that can be inserted in the body to treat diseases or perform a surgical procedure. Both terms are used interchangeable in the following if not stated otherwise.

By modifying the material or adjusting the way cannulas/catheters are manufactured, it is possible to tailor them for cardiovascular, urological, gastrointestinal, neurovascular, and ophthalmic applications. A catheter/cannula left inside the body, either temporarily or permanently, may be referred to as an “indwelling catheter/cannula” (for example, a peripherally inserted central catheter/cannula).

Catheters and cannulas may be inserted into a body cavity, duct, or vessel. Functionally, they allow drainage, administration of fluids or gases, access by surgical instruments, and/or also perform a wide variety of other tasks depending on the type of catheter or cannula. The process of inserting a catheter is “catheterization”. The process of inserting a cannula is “cannulization”. In most uses, a catheter/cannula is a thin, flexible tube (“soft”) though catheters/cannulas are available in varying levels of stiffness depending on the application.

It is an object of the invention to disclose a cannula system that is easy to use and that especially reduces the risk of injuries during insertion and or removal of catheters/cannulas. Furthermore, it is an object of the invention to disclose corresponding methods.

This object is solved by a cannula system according to claim 1. Embodiments are disclosed in the sub claims. Furthermore, the object is solved by a method according to the independent method claim. Embodiments are disclosed in the sub claims.

The proposed cannula system comprises:

a first cannula, preferably an outer cannula and/or preferably an elongated first cannula, having a minimum inner diameter or width,

a second cannula, preferably an inner cannula and/or preferably an elongated second cannula, having a maximum outer diameter or width, wherein the minimum inner diameter or width is greater than the maximum outer diameter or width,

wherein the second cannula is adapted to be guided into the first cannula, preferably if the first cannula is already inserted into a body of a subject, such that a first lumen of the first cannula remains in the first cannula between an outer surface the second cannula and an inner surface of the first cannula, and wherein the first lumen of the first cannula defines a first fluid conduit and a first lumen of the second cannula defines a second fluid conduit.

Thus, it is possible to perform for instance medical treatments with artificial blood circulations thereby preventing trauma due to successive insertion of both cannulas of a dual cannula or of the cannulas of a multi cannula system.

The first fluid conduit may be a conduit for a liquid. The second fluid conduit may also be a conduit for liquid. However, other kind of fluid conduits may also be used, for instance for a conduit for a gas.

The cannulas may comprise an internal metal scaffold or framework that is for instance spirally wounded. However other frameworks or no framework are also possible. The material of the cannulas may be biocompatible and/or comprise or consist of urethane and/or silicone and/or polyvinyl chloride.

The cannulas may be introduced into a body of a subject, e.g. a human patient, along a guide wire. Snares may be used for positioning the guide wire and or for advancing one of the cannulas. Seldinger techniques may be used for instance.

A first introducer may be used for introducing the first cannula. A second introducer may be used for introducing the second cannula. The introducer may leave only a small gap or offset to the inner surface of the respective cannula preventing or reducing blood flow out of the cannula during the placement of the cannula. A guide wire may also guide the introducer, e.g. the introducer may have a longitudinal central opening for a guide wire.

In this application document the definition for “distal” is far from a person that inserts the cannula/catheter. “Proximal” means near to the person that inserts the cannula/catheter. In the following the longitudinal axis of lumen portion or the extension thereof beyond the lumen portion may be used as a reference axis. The terms “radial”, “axial” and/or “angularly” may be used with regard to this reference axis, similarly to cylinder coordinates are used in a cylindrical coordinate system.

Apertures may be arranged on sidewalls of the first cannula, preferably at the distal end portion. Alternatively, the first cannula may have a first tip that may comprise apertures in its side wall. Furthermore, apertures may be arranged on sidewalls of the second cannula or within sidewalls of separate tip that is mounted on distal end of second cannula. “Sand blasting effect” on vessels may be prevented by using multiple apertures at one or at a single injecting or infusing cannula. Multiple apertures may also be useful on a cannula that is used as an outlet because clogging of apertures may be prevented or if some of the apertures are clogged by surrounding tissue other apertures may still be open.

The first fluid conduit and the second fluid conduit may be separate conduits, no direct fluid transfer may be possible between first fluid conduit and the second fluid conduit. The first fluid conduit and the second fluid conduit may be connected only via a circuitry that is outside of the body, for instance a circuitry that comprises flexible tubes and/or at least one pump and/or medical devices like an oxygenator, filter etc. The first fluid conduit may be usable for guiding the first fluid through the first cannula. The second fluid conduit may be usable for guiding the first fluid or the second fluid through the second cannula.

The cannula system may be used for lung assist, for only left lung lobe assist or for only right lung lobe assist. Lung assist may refer to an enrichment of oxygen content of blood and/or to a reduction of the content of carbon dioxide. Further, the cannula system may be used for lung perfusion, for only left lung lobe perfusion or for only right lung lobe perfusion. Perfusion may refer to a process in which a fluid flow through an organ is isolated completely or almost completely from other fluid flows in the same body. Almost completely may mean that less than 10 percent of volume or less than 5 percent of volume of the perfused fluid mixes with other fluid flows of the body. The following diseases may be treated: chronic obstructive pulmonary disease (COPD), acute respiratory distress syndrome (ARDS), pulmonary arterial hypertension (PAH) etc.

The cannula system may be used for heart assist, for only left heart assist or for only right heart assist. The following diseases may be treated: right ventricle failure (RVF), left ventricle failure (LVF), etc.

Furthermore, the cannula system may be used for kidney assist or perfusion, for liver assist or perfusion or for other organs assist or perfusion. The cannula system may be used to bridge the time for patients on waiting list to organ transplantation (BTT), especially lung or heart transplantation or other organ transplantations.

The cannula system may be a dual lumen cannula system or a multiple lumen cannula system comprising more than two lumens for guiding a fluid. The cannula system may be used also advantageous if no diameter variable arrangement, e.g. cage arrangement, is used and/or if no membranes on the diameter variable arrangement are used.

However, the usage of at least one diameter variable arrangement may promote the usage of a stepwise introduction of the cannulas, i.e. first insertion of first cannula and after complete insertion of the first cannula insertion of the second cannula. As far as the words “diameter variable arrangement” (DVA) are used in this application they shall refer to an expandable arrangement that has a first embraced volume in a non-expanded state and a second embraced volume in the expanded state. The second volume may be greater than 2, 3, 4, 5 etc. times the first volume. The diameter may increase also by at least factor 2, 3, 4 or more comparing the non-expanded state and the expanded state. However, the second volume may be smaller than 100 times the first volume. The diameter in the expanded state may be smaller than 20 times or smaller than 10 times the diameter in the non-expanded state, for instance using the maximum diameter in the respective state.

Radiopaque markers or other marker techniques may be used to ease placement of the distal tips of both cannulas. Radiopaque markers may be visible in a CT (Computer Tomography) or MRT (Magneto Resonance Tomography).

The first lumen of the first cannula may be limited by an outer surface of the second cannula such that fluid guidable in the first lumen of the first cannula may be in physical contact with the outer surface of the second cannula. This feature may be realized along a length that is equal to the axial length of a portion of the first cannula in which the second cannula is arranged within the first cannula, or at least 90 percent of the axial length of this portion. This means that no further separating element is used between the first cannula and the second cannula. Thus, it is possible to have a cannula system with low bending stiffness.

The first cannula may be flexible and bendable such that it is insertable into a vessel of the body, preferably into a blood vessel. The first cannula may be flexible and bendable such that it is guidable into the internal jugular vein, the subclavian artery or the subclavian vein, thereafter into the superior vena cava and then into the right atrium preferably at least up to or through the atrial septum. This may allow mobility of a patient even if the cannula system is still in his or her body. However, it is also possible to use femoral access.

Alternatively, the first cannula may be bendable such that it is insertable intravascular to the superior vena cava and up to the right atrium or up to the right ventricle.

The unit of bending stiffness is N mm\hat{ }2 (newton square millimeter). The bending stiffness may be calculated according to the following equation:

bending stiffness=Young's module E*second moment of area I.

The second moment of area may have the unit m\hat{ }4, i.e. (meter)\hat{ }4, for instance.

Young's module is defined as:

E=sigma/epsilon,

wherein sigma is the uniaxial stress=F/A=force/area and epsilon is the strain or proportional deformation=delta length/original length. The unit of Young's module may be MPa or N/mm². It may also be practicable to measure the bending stiffness by an appropriate method in order to classify the cannula system for specific applications. Another possibility is to refer to the outer diameter of the cannulas, for instance using the unit French, i.e. 1 French equal to about 3 Millimeters. A greater value of the diameter may also result in a higher bending stiffness.

A portion of the first cannula through which the second cannula is guidable within the first cannula may have a length of at least 20 cm (centimeter) or at least 40 cm. The length of the common or overlapping portion may be at most 60 cm length. These length values may be valid for adult persons having a body height in the range of 160 cm to 200 cm (centimeter). The second cannula may be guidable beyond a distal end of the first cannula by at least 5 cm or by at least 10 cm. These length values may be valid for adult persons having a body height in the range of 160 cm to 200 cm (centimeter). Thus, the cannula system may be usable for heart surgery. These length may be adapted for child body heights between 140 cm and 160 cm

The first cannula and/or the second cannula may be pre-bended by an angle within the range of 60 degrees to 175 degrees or within the range of 70 to 145 degrees. Exemplary values are: 75 degrees, 90 degrees and 135 degrees and angles within the range of plus or minus 5 degrees around these values. These angles may ease an insertion of the second cannula or of both cannulas through the septum of the heart, preferably through the atrial septum coming from internal jugular vein or subclavian vein through the right atrium. The pre-bending of both cannulas may be at a common position if both cannulas are placed at their final destinations. Alternatively, the pre bending may be only at the second cannula within a portion that is outside of the first cannula if both cannulas are arranged in their final positions. Furthermore, it is possible to pre bend only the outer cannula at a position at which the inner cannula (second) is not bend if both cannulas are in their final positions. Pre bending may facilitate the advancement of the cannula around “corners” or around angles that are greater than 45 degrees within the body. This may be preferable at bifurcation points of vessels or if vessels open out into cavities, for instance the vena cava into the right atrium of the heart.

According to a further embodiment, the first cannula may comprise a first coupling port, wherein the first coupling port may be adapted to be coupled or may be coupled to a blood pump, preferably to an inlet port of the blood pump or to an outlet port of the blood pump. Alternatively and/or additionally, the second cannula may comprise a second coupling port, wherein the second coupling port may be adapted to be coupled or may be coupled to a blood pump or to the blood pump, preferably to an inlet of the blood pump or to an outlet of the blood pump. The blood pump may be a membrane pump or other type of pump. Thus, it is possible to perform medical treatments with artificial blood circulations thereby preventing trauma due to successive insertion of both cannulas of a dual cannula.

The cannula system may comprise an opening in the outer wall of the first cannula through which the second cannula is insertable into the lumen of the first cannula. The opening may be located within a sidewall of the first cannula or the opening may be located at a proximal end of the first cannula, preferably centrally with respect to a longitudinal axis of the first cannula. Other positions of the opening are possible as well. The insertion of the second cannula into the first cannula at the proximal end portion of the cannula system may be realized without a separate connector. However, in other embodiments a separate connector portion may be used to connect proximal ends or portions of the first cannula and of the second cannula mechanically but not fluidly.

There may be at least one sealing element that is arranged between the first cannula and the second cannula, preferably arranged within the opening. The sealing element may comprise or consist of a retaining ring, a sealing ring or a gasket. Appropriate materials for the sealing element may be softer than the material of the cannulas. Rubber may be used as a material for the sealing element, e.g. natural rubber (caoutchouc) or synthetic rubber. An O-ring seal or sealing is the simplest sealing element that may be used. Furthermore, a multi-flap valve or another self-sealing member may be used what is explained below in more detail, i.e. for instance two flexible membranes.

The cannula system may comprise a distal closure element that prevents the passage of fluid through the distal end of the outer cannula beyond the closure element into the first lumen of the first cannula. The closure element may be arranged on a distal end portion of the first cannula. The closure element may be adapted to be in a closed state or in an at least partially opened state if the second cannula and/or an introducer is arranged within an opening of the closure element. The introducer may be used to introduce the first cannula into the body or to introduce the second cannula. If the introducer is used to introduce the first cannula it may also be used to stretch a diameter variable arrangement that is detached or mounted on a distal end portion or another portion of the first cannula but beyond the closure element. The distal closure element may comprise at least one sealing membrane, preferably at least one sealing membrane comprising an aperture, and/or at least one self-sealing flap. The distal closure element may also prevent flow of fluid out of the first lumen through distal tip of the first cannula if the first cannula is used as an inlet. Thus, at least one sealing membrane with an aperture or a sealing membrane without an aperture may be used. In the latter case the membrane may be pierced by the second cannula, an introducer or an auxiliary tool. Furthermore, a multi-flap valve or another self-sealing member may be used what is explained below in more detail, i.e. for instance two flexible membranes.

The cannula system may comprise a first introducer for introducing at least the first cannula and a second introducer for introducing the second cannula. The second introducer may be longer than the first introducer, preferably by at least 5 cm or at least 10 cm. Furthermore, the second introducer may be thinner than the first introducer allowing the diameter of both introducers to be optimized for the different inner diameters/inner width of both cannulas. The second introducer may be at most 30 cm longer than the first introducer. The introducer may have further functions in addition to ease forwarding of the respective cannula. Thus, the first introducer and/or the second introducer may be used to insert at least one diameter variable arrangement, for instance to hold them in a stretched state, i.e. with small diameter. If and when introducer is pulled back, the diameter variable arrangement may self-expand. A guide wire may be used that may extend also through the introducer, e.g. its longitudinal axis.

The first cannula may be configured such that the second cannula is arranged coaxially within the first cannula if inserted into the first cannula, preferably along the whole length of an overlapping region of the first cannula and of the second cannula or along at least 90 percent of the length of the overlapping region. The first cannula may comprise at least one supporting element at its inner surface and/or the second cannula may comprise at least one supporting element at its outer surface. The at least one supporting element may be adapted to radially support a part of the second cannula relative to the first cannula to maintain the first cannula and second cannula in a predetermined relative arrangement with respect to one another. Coaxially may mean in the center of the first cannula. The supporting element may protrude radially inwardly from an inner surface of the outer cannula (first cannula) or radially outwardly from an outer surface of the inner cannula (second cannula). At least three supporting elements may be circumferentially distributed evenly at a specific axial position of the first cannula. Thus, the outer surface of second cannula may be supported with distance to inner surface of first cannula. This may guarantee that the fluid is in movement at all positions of the first lumen of the first cannula and/or that shearing forces that may destroy blood cells are reduced.

However, alternatively, the first cannula may be formed such that the second cannula is arranged non-coaxially within the first cannula if inserted into the first cannula. The second cannula may be arranged loosely within the first cannula, preferably along the whole length of the overlapping length of the first cannula and the second cannula if the second cannula is completely inserted or along at least 90 percent of the overlapping length of both cannulas. No additional mounting elements within first cannula will be necessary resulting in a simple manufacturing process.

The first cannula may have a circular or oval inner cross section and the second cannula may have a circular or oval outer cross section, preferably along its whole length or along at least 90 percent of the length of the second (outer) cannula, e.g. axial length.

The cannula system may comprise a first diameter variable arrangement mounted/attached on the first cannula, preferably on a distal end portion of the first cannula. The first diameter variable arrangement may comprise a first cage arrangement. The cage arrangement may comprise wires or stripes that may be arranged without crossing each other in the portion that has a variable diameter. The wires or stripes may be arranged nearly in parallel when in the non-expanded state i.e. during introducing of the first cannula and of the first diameter variable arrangement therewith. The diameter variable arrangement may be made of a different material compared to the material of the first cannula. However, it is also possible that the first cannula “extends” into the diameter variable arrangement from a portion that has a diameter that is not variable in the sense that the cross section may be made greater, for instance may be made greater by at least factor two or at least factor three. A preferred material for the wires is a shape memory alloy (SMA) or material, for instance a material that changes its shape depending on the temperature of the material. Nitinol (Nickel Titanium Naval Ordnance Laboratory) is an example for such a material. However, other materials may also be used, for instance NiTi (nickel titan), NiTiCu (nickel titan copper) CuZn (copper zinc), CuZnAl (copper zinc aluminum), CuAlNi (copper aluminum nickel). Further materials that may be used are super elastic materials, stainless steel wire, cobalt-chrome alloys or cobalt-chromium-nickel-molybdenum-iron alloy. Further materials that may be used are super elastic materials, stainless steel wire, cobalt-chrome alloys or cobalt-chromium-nickel-molybdenum-iron alloy. The thickness and/or diameter of the wires may be in the range of 0.1 mm (millimetre) to 2 mm, especially if only three or four wires are used within the expandable arrangement that may also be named as cage arrangement. The thickness and/or dimeter of the wires may be in the range of 0.1 mm (milli meter) to 1 mm or in the range of 0.25 mm to 0.75 mm. Thinner wires may be useful if more than four wires are comprised within the cage arrangement.

The first diameter variable arrangement may have an expanded state having a maximum outer diameter that is greater than the maximum outer diameter in a non-expanded state of the diameter variable arrangement, preferably greater by at least factor two or at least factor three. The factor may be less than factor 50 or less than factor 10.

The same features that apply to the first diameter variable arrangement may also apply to a second diameter variable arrangement that may be mounted/attached on the second cannula. There are at least three possibilities for realization, i.e. only one diameter variable arrangement on the first cannula or on the second cannula, or two diameter variable arrangements on the cannula system, i.e. one on the first cannula and one on the second cannula. In the latter case the second diameter variable arrangement is inserted through the first diameter variable arrangement, e.g. second cage arrangement through first cage arrangement. It may be easier to insert for instance a cage arrangement through another cage arrangement if some of the cage wires are omitted depending preferably on the desired direction of forwarding of the second cannula/second cage arrangement.

The non-expanded state may be a state in which the respective diameter variable arrangement can be introduced into the body and/or into the first cannula, preferably using an introducer that stretches the diameter variable arrangement. The diameter variable arrangement may have one or several of the following functions: fixation, holding of membrane, preventing that a sidewall of the vessel closes drainage openings, weakens sand blasting effect for injection cannulas, etc.

The lumen may be adapted to guide an introducer member that preferably comprises or consists of an elongated structure, e.g. a long rod. In the case of a split tip cannula a split tip introducer member may be used or at least two separate introducer members. The expandable arrangement may comprise a contact area that is adapted to have mechanical contact with the introducer member. In the expanded state, preferably also in the non-expanded state, the contact area may overlap with an opening of the lumen as seen in top view onto the opening along a longitudinal axis defined by the lumen. The contact area may be opposite to an opening of the lumen. The expandable arrangement may be configured such that it changes from the non-expanded state to the expanded state if the introducer member is moved away from the contact area. The expandable arrangement may be configured such that it changes from the expanded state to the non-expanded state if the introducer member makes contact to the contact area.

The diameter variable arrangement may be different from a cannula that is diameter variable along its entire length or almost its entire length, i.e. along at least 90 percent of its length.

The first cage arrangement and/or the second cage arrangement may comprise a plurality of cage wires that are easy to fabricate and/or easy to pre bend. There may be 2 to 15 cage wires, preferably 3 to 12 cage wires, i.e. 3, 4, 5, 6, 7, 8, 9, 10, 11 or 12 cage wires.

One or each of the cage arrangement may comprise in the expanded state of the cage arrangement:

a first or proximal portion in which the distance between neighboring wires increases with increasing distance to a mounting portion of the wires,

an optional transition portion wherein distance between neighboring wires is constant with increasing distance to the mounting portion of the wires,

and a third or distal portion in which the distance between neighboring wires decreases with increasing distance to the mounting portion of the wires.

The first cage arrangement and/or the second cage arrangement may comprise a backwardly bended portion which follows the distal portion, especially if along the course of the wires.

Within the backwardly bended portion, the wires may change direction and/or neighboring wires may have decreasing distances with decreasing distance to the mounting portions of the wires. The expandable arrangement may comprises a cage tip portion following the backwardly bended portion, wherein in the cage tip portion the cage wires are connected with each other. There may be a backward bending by more than 90 degrees, by more than 110 degrees, by more than 120 degrees, or more than 140 degrees up to for instance 180 degrees. Alternatively, the expandable arrangement may not comprise a backwardly bended portion. Additionally to the backwardly bended portion, there may be a radially extending straight portion in which the wire extends only radially inward at the same axial position for at least 1 mm, at least 2 mm, at least 3 mm, at least 4 mm or at least 5 mm.

The wires may not have angular overlap between two neighboring wires within each portion and also over all of the portions thus reducing the outer diameter in the stretched or insertion state. The wires may not cross each other in free portions, crossing may be possible only within a proximal mounting portion of the wires or at the distal end connecting portion. This arrangement of the wires is different form an interwoven or braided material.

The proximal ends of the cage wires of the first cage arrangement on the first cannula may be wound around an outer surface of the first cannula, preferably around a distal end portion of the first cannula. The same features may be valid for the second cage arrangement with regard to the second cannula. To coil up wires is a simple but efficient technology for mounting the cage arrangement. Further, the diameter is not increased significantly.

The distal ends of the cage wires may be connected with each other, preferably using a connecting element and/or by twisting them together. Again, a simple technique is used. Furthermore, it may be easier to fasten the distal ends of the wires on each other than on the first cannula or on the second cannula. Moreover, the distal ends may be arranged on a longitudinal or central axis of the respective cannula allowing the usage of a straight introducer to stretch the respective cage arrangement during insertion.

The cage wires may be distributed angularly such that, in a given axial position, for two different pairs of neighboring cage wires the wires forming the pair have respectively a first distance relative to each other which is equal for both pairs and that neighboring wires forming yet another pair of neighboring wires have a second distance relative to each other that is greater than the first distance, preferably equal to or greater than twice the first distance. The same may apply for the respective angular distances. This may mean that one wire, two consecutive wires or three consecutive wires may be omitted in order to allow the insertion of a further cage through the cage that has the omitted wires. Preferably, wires are omitted on the cage arrangement on the first cannula to allow the insertion or forwarding of the second cannula with or without second cage arrangement through the first cage arrangement.

The cage arrangement may be configured in the expanded mode or state as a sphere or as an ellipsoid. The sphere may have the same radius in all directions in the expanded state. The ellipsoid may have three main axis that have different length or at least one of the main axis may be longer than the other two main axis in the expanded state.

The first diameter variable arrangement may comprise a first membrane. The first membrane may be folded or less stretched in the non-expanded state of the first diameter variable arrangement, e.g. it may have lots of wrinkles. The wrinkles may be arranged between supporting structures or outside of spaces that are arranged between such supporting structures, e.g. wires. The first membrane may be expanded in the expanded state of the first diameter variable arrangement. The same features may apply to a second membrane that may be part of the second diameter variable arrangement. The membrane may be connected to the diameter variable arrangement using several techniques, for instance dip molding, plastic welding, sewing and/or using glue. The connection technique may also make a fluid tight connection to the cannula or to a mounting part of the diameter variable arrangement on the cannula.

The material of the membrane may be fluid tight or liquid-tight (impermeable) in both directions, i.e. from inside of diameter variable arrangement (cage) to the surrounding area and/or from surrounding area to inside of cage. Additionally or alternatively, the membrane may define a volume which is fluidly connected to the lumen but with a greater diameter than the lumen, especially in the expanded state of the diameter variable arrangement (expandable arrangement). The membrane may be fluid tight and the opening may be an inlet into the lumen or an outlet out of the lumen.

The membrane may be or comprise a thin sheet of material, having for instance a thickness of less than 0.5 millimeters, less than 100 micrometer or less than 50 micrometer (micron). However, the membrane may be thicker than 10 micrometer in order to provide appropriate strength. Polytetrafluoroethylene (PTFE) may be an appropriate material for the membrane, preferably if it is manufactured by electro spinning, for instance within an electrical field. However, other materials may also be used. Only one sheet of membrane may be arranged for forming the membrane, e.g. there may be only one layer of membrane material.

It is possible to insert the second diameter variable arrangement and/or the second membrane of the second diameter variable arrangement through the first diameter variable arrangement or through an opening of the first membrane. Parts of the first membrane may be omitted to have room for the forwarding of the second diameter variable arrangement and/or the second membrane. It is possible to use membranes if three or more cage wires are used, for instance 4 or more than 4 cage wires.

In the expanded state of the first diameter variable arrangement, an edge of the first membrane may define an opening that faces distally relative to the first cannula, i.e. to a longitudinal axis of the first cannula. The same may apply to the second membrane. This may mean that an edge of the membrane delimits the edge of the opening along the entire circumference of the opening. Preferably the membrane may extend circumferential around the longitudinal axis of the first cannula or the second cannula by at least 300 degrees or by at least 360 degrees. Additionally, the membrane may extend from a proximal end of the diameter variable arrangement at most three quarter or at most half way or at most one quarter to a distal end of the diameter variable arrangement. Thus, the membrane may have a lateral sealing function and/or a function in directing an inlet flow or an outlet flow.

A membrane having a distally facing opening may have, especially in the expanded state, the following relations relative to the portions of the cage arrangement that are mentioned above:

the proximal portion and/or the optional transition portion may be covered by the membrane, and

the distal portion and/or the optional transition portion may not be covered by the membrane.

A part of the inner volume of the diameter variable arrangement, for instance of the cage arrangement may be separated from the surrounding area by the membrane. The surroundings may be fluidly connected through the opening of the first membrane to the first conduit or to an opening of the second membrane to the second conduit.

Alternatively, in the expanded state of the first diameter variable arrangement, an edge of the first membrane may define an opening that faces laterally relative to the first cannula, i.e. to a longitudinal axis of the first cannula. The same may apply to the second membrane. If the cannula system comprises two membranes there are in principle the following possibilities for the openings: two distal facing opening, two laterally facing openings, a distally facing opening on the first cannula and a laterally facing opening on the second cannula, or a laterally facing opening on the first cannula and a distally facing opening on the second cannula.

Again, an edge of the membrane may delimit the edge of the opening along the entire circumference of the opening. The membrane may extend circumferential around the longitudinal axis or around an extension of the longitudinal axis of the first cannula or the second cannula by less than 270 degrees or by less than 200 degrees. The membrane may cover the cage arrangement from 0 degree to 180 degrees or from 0 degree to 210 degrees or from 0 degree to 240 degrees. Additionally, the membrane having the laterally facing opening may extend from a proximal end of the diameter variable arrangement up to a distal end of the diameter variable arrangement or along at least 80 percent of this distance. The distance may be measured in the non-expanded state or in the expanded state and/or may be an axial distance.

A membrane having a laterally facing opening may have, especially in the expanded state, the following relations relative to the portions of the cage arrangement that are mentioned above:

the proximal portion, the optional transition portion, and the distal portion may be covered by the membrane on a first side of the cage arrangement, and

the proximal portion, the optional transition portion and the distal portion may not be covered by the membrane on a second side of the cage arrangement that may be opposite to the first side of the cage arrangement.

Again, a part of the inner volume of the diameter variable arrangement (cage) may be separated from the surrounding area by the membrane that has a laterally facing opening. The surroundings may be fluidly connected through the laterally facing opening to the first conduit or to the second conduit.

The cannula system may comprise at least one fixation element that is configured to prevent an axial movement of the second cannula relative to the first cannula after complete insertion of the second cannula, wherein preferably the fixation element is arranged on an outside of the first cannula and/or on an outside of the second cannula. It may be easier to have the fixation element outside of the first cannula and also outside of the second cannula for instance with regard to easier operation and/or easier design. However, the fixation element may also be arranged partially or completely inside of the first cannula.

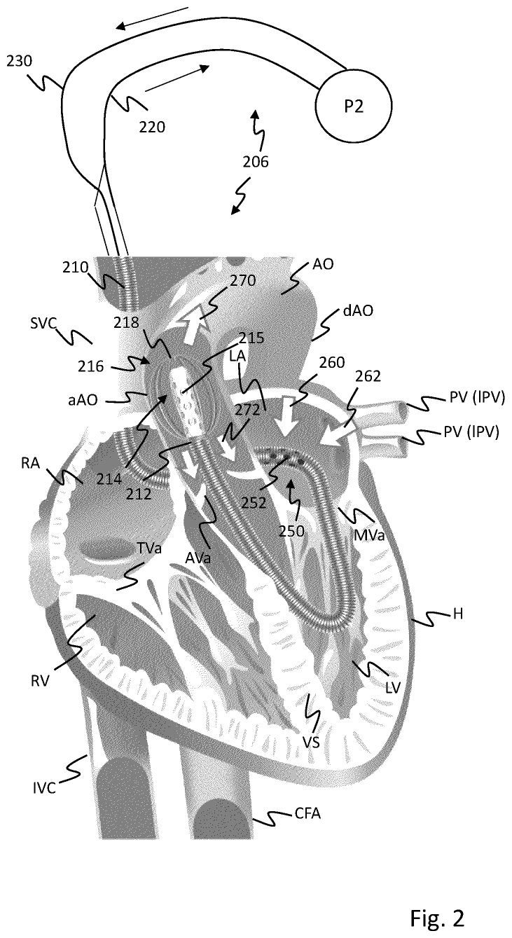

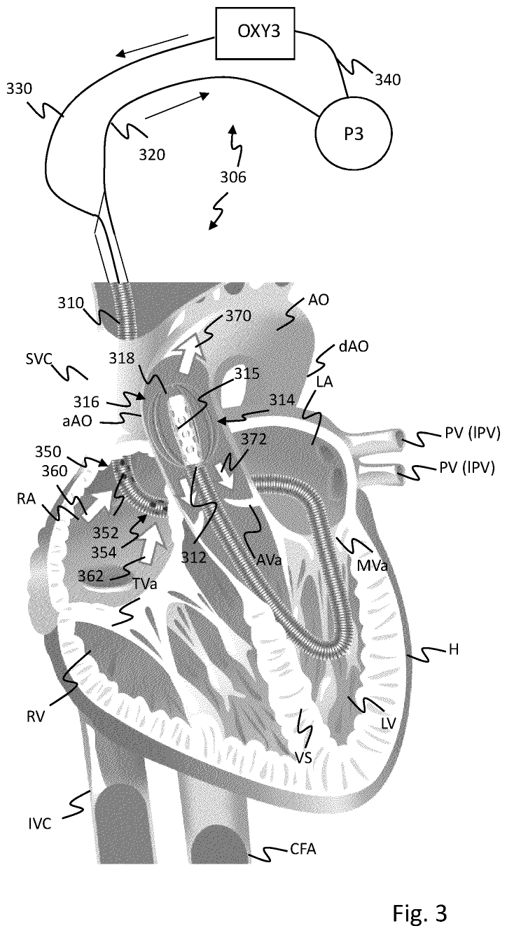

According to FIG. 2 or 3, for left and bi ventricle assisted devices:

the first cannula may be adapted to be inserted intravascular, preferably jugular, through the superior vena cava into an interior region of the heart. The second cannula may be adapted to be inserted through the first cannula into an interior region the heart, preferably to a different interior region of the heart compared to the interior region where the first cannula is positioned, or into the aorta,

preferably a first diameter variable arrangement may be mounted or arranged on the distal end portion of the first cannula,

the second cannula may be adapted to be inserted further into the left atrium, the left ventricle and into the aorta, preferably into the ascending aorta, and

preferably a second diameter variable arrangement may be mounted on the distal end portion of the second cannula.

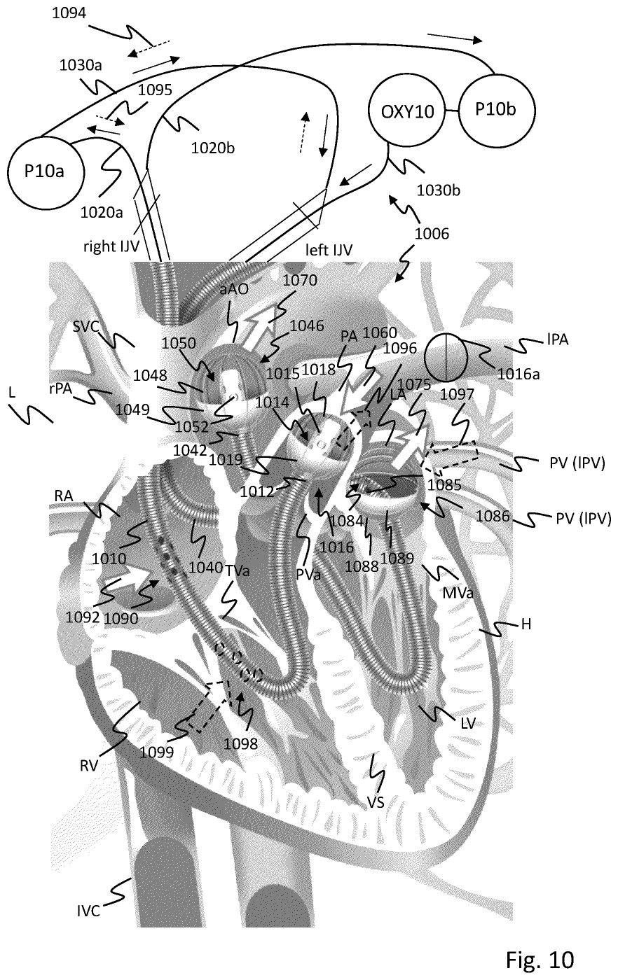

According to the three variants of FIG. 10, for lung perfusion:

the first cannula may be adapted to be inserted intravascular through the vena cava, preferably through the superior vena cava, into the left atrium,

and the second cannula may be adapted to be inserted through the first cannula, through the left atrium, through the left ventricle to the aorta, preferably up to the ascending aorta,

a first diameter variable arrangement may be mounted on the distal end portion of the first cannula, preferably covered with a first membrane, wherein the first membrane in an expanded state of the first diameter variable arrangement preferably has an opening facing laterally relative to the first cannula and/or an edge that is essentially parallel to two cage wires of the first diameter variable arrangement,

and a second diameter variable arrangement may be mounted or arranged on the distal end portion of the second cannula, preferably covered with a second membrane, wherein the second membrane in an expanded state of the second diameter variable arrangement preferably has an opening facing distally relative to the second cannula and/or an edge that is essentially transversally to cage wires of the second diameter variable arrangement.

The pump direction may be changed once or more than once to flush the lung.

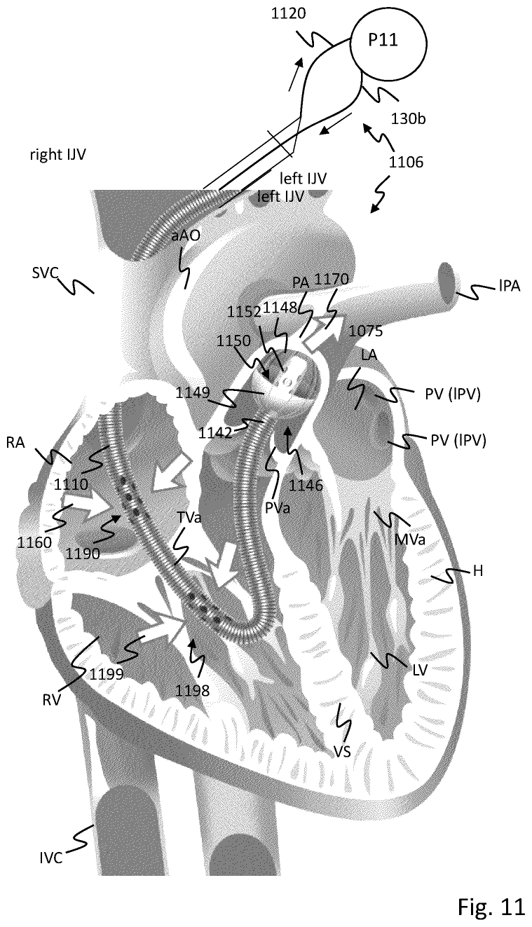

According to FIGS. 10 and 11, for lung assist or right ventricle assist:

the first cannula may be adapted to be inserted intravascular into an interior region of the heart and the second cannula may be adapted to be inserted through the first cannula into the pulmonary artery, into the left pulmonary artery, into the right pulmonary artery and/or into the lung,

wherein preferably the first cannula may be adapted to be inserted intravascular through the vena cava, preferably through the superior vena cava, up to the right atrium or up to the right ventricle,

preferably a first diameter variable arrangement may be mounted or arranged on the distal end portion of the first cannula,

preferably a second diameter variable arrangement may be mounted or arranged on the distal end portion of the second cannula, preferably covered with a membrane, wherein the membrane in an expanded state of the diameter variable arrangement preferably has an opening facing distally relative to the second cannula and/or an edge that is essentially transversally to cage wires of the diameter variable arrangement.

The pump direction may be changed once or more than once to flush the lung.

A further aspect relates to an assembly of a cannula system according to one of the preceding embodiments and of a blood pump, preferably of a membrane pump. The blood pump may be fluidly connected to the first cannula and to the second cannula. Thus, it is possible to perform medical treatments with artificial blood circulations thereby preventing trauma due to successive insertion of both cannulas of a dual cannula.

The membrane pump may comprise at least one variable volume reservoir that is adapted to have an aspiration operating phase for drawing fluid, preferably a liquid, e.g. blood, into the variable volume reservoir and that is adapted to have an expulsion operating phase for pressing the fluid out of the variable volume reservoir. The variable volume reservoir may be fluidly connected to the first cannula and to the second cannula. The membrane pump may be operated in a pulsatile operation mode, preferably synchronous or depending on the heartbeat of a patient. Thus, high pumping values may be reached without damaging or destroying blood cells. 60 heartbeats per minute and 100 ml per pumping cycle would give 6 liter per minute as an example.

The methods that are described in the following may be performed using the cannula system that is described above. Therefore, the same technical effects that are valid for the cannula system and its embodiments may also be valid for the methods. Vice versa, technical effects that are valid for the methods may also be valid for the cannula system and its embodiments.

The method for cannulizing a subject comprises:

inserting a first cannula or outer cannula into a body of the subject,

after insertion of the first cannula, guiding a second cannula or inner cannula through or along the first cannula into the body of the subject,

whereby a first lumen of the first cannula is left outside of the second cannula,

and thereafter, guiding a first fluid through the first lumen of the first cannula and guiding the first fluid or a second fluid through a first lumen of the second cannula.

Thus, a multi lumen cannula, especially a dual lumen cannula is proposed, that allows separate insertion of the first cannula and of the second cannula. This separated insertion has many technical effects that may be advantageous for several medical and nonmedical applications. An advantage of dual lumen cannula is that only one dissection is necessary and only one punctuation of vasculature.

The surgery may be heart surgery and/or lung surgery or surgery of other organs, for instance kidney, liver etc. The first cannula may be inserted through a vein or artery. When the second cannula is inserted through the first cannula there will be no friction between the second cannula and the vasculature as long as the second cannula is only within the first cannula. The same is valid, if the first cannula is inserted through tissue of the body that is not part of blood vasculature. Reference is made to the disease that are listed above for the cannula system.

Further, the first cannula is first introduced into the body without comprising the second cannula. Thus, the bending stiffness of the first cannula may be lower than the bending stiffness of the system comprising the first cannula and the second cannula if the second cannula is inserted into the first cannula. This effects that the introduction of the first cannula may be made easier compared to an introduction of a dual lumen cannula that does not allow separate insertion of first cannula and second cannula. Often, it may be an advantage if the cannula is more flexible if is forwarded along a guide wire. Only if the first cannula has been forwarded to its final destination within the body or at least near to its final destination it may be comparably easy to forward the second cannula within the first cannula. Thereby, the guide wire may be used again or a second guide wire may be used.

Furthermore, the second cannula may have a smaller maximum width/diameter than the first cannula, for instance the second cannula may have a smaller maximum outer width/diameter than a maximum outer width of the first cannula and/or than a minimum inner width of the first cannula. If the second cannula extends over a distal end of the first cannula greater curvature, i.e. smaller radius of curvature, may be possible compared to other dual lumen cannulas.

When the cannula system is removed it is preferred that the second cannula is removed completely or almost completely out of the body, for instance at least 90 percent of the length that was inserted into the body. Thereafter the first or first cannula is removed. Again, there is no friction between the second cannula and the vasculature or tissue of the body in the portion that is protected by the first cannula. Furthermore, the first cannula is not as stiff if the second cannula is removed. This makes removal of the first cannula easier and the risk of injuries is reduced. Removal of the cannula system may take place after some hours, for instance after less than 10 hours, or after days, for instance after less than 5 days, or after weeks, for instance after at least one week or at least two weeks.

The first fluid may be a liquid. The second fluid may also be a liquid. However, other kind of fluids may also be used, for instance a gas.

The first fluid may be transported from outside of the body through the first lumen of the first cannula into the body. Alternatively, the first fluid may be transported from within the body through the first lumen of the first cannula to the outside of body. The first fluid or the second fluid may be transported from outside of the body through the first lumen of the second cannula into the body. Alternatively, the first fluid or the second fluid may be transported from the inside of the body through the second lumen of the second cannula to the outside of the body. However, alternatively, fluid transport may be only within the body, for instance using natural pressure differences or internal pump.

The flow in the first lumen of first cannula may have the same direction as the flow in the first lumen of the second cannula. Alternatively, the flow in the first lumen of the first cannula may have a different direction compared to the direction of the flow in the first lumen of the second cannula.

The first lumen of the first cannula may be used for the injection of blood or another fluid into the body and the second cannula may be used for removal of blood or another fluid from the body. Alternatively, the first lumen of the first cannula may be used for removal of blood or another fluid from the body and the second cannula may be used for the injection of blood or another fluid into the body.

The first lumen of the first cannula may also be defined by a first surface of the second cannula after insertion of the second cannula. Blood or other fluid in the first lumen of the first cannula may be in physical contact with first surface of second cannula.

The first cannula may be inserted along a vessel of the body, preferably along a blood vessel. The first cannula may be inserted into the body along a length of at least 20 cm (centimeter) or of at least 30 cm, especially through the right jugular vein.

If the left jugular vein is used, the first cannula may be inserted into the body along a length of at least 35 cm, 40 cm, 45 cm, 50 cm, 55 cm, 60 cm, 65 cm or of at least 70 cm. The second cannula may be inserted into the body along a length that is at least 5 cm, 10 cm, 15 cm, 20 cm, 25 cm or 30 cm longer than the length along which the first cannula is inserted into the body. The blood vessel may be an artery (blood flows is away from the heart) or a vein (blood flow is directed to the heart). The length of the first cannula and/or of the second cannula may be smaller than 60 cm (centimeter). The subject may be an adult, i.e. a woman or a man. However, the subject may also be a child. The method may be especially useful for smaller vasculature because this vasculature has smaller diameters and a smaller radius of curvature may be necessary.

The first cannula may be bended by an angle within the range of 60 degrees to 175 140 degrees or within the range of at least 70 degrees to 145 degrees. This may be especially important for heart surgery, for instance in order to ease puncturing of the atrial septum or of the ventricle septum. This bending may be less than 180 degrees. Examples for the angle are 75 degrees, 90 degrees or 135 degrees.

The first fluid may be guided into a first direction within the first lumen of the first cannula and the first fluid or the second fluid may be guided into the same or into an opposite direction compared to the first direction within the first lumen of the second cannula. The first fluid and/or the second fluid may comprise the same fluid, preferably blood or a fluid comprising blood. Alternatively, the first fluid and/or the second fluid may be different fluids. The fluid may be only blood or blood combined with a medicament or only medicament in an appropriate fluid that may be different from blood. Furthermore, components of blood may be comprised within the first fluid or the second fluid, for instance red blood cells and/or blood plasma. The fluids may be saline or blood that is diluted with saline.

This allows to establish circular flows with forward and back branches using only one dual lumen cannula. However, if an additional single lumen cannula or dual lumen cannula is used within the same body it is possible to establish flows flowing in the same direction within the first cannula and the second cannula of the dual lumen cannula system that allows separate insertion of the first cannula and of the second cannula.

The second cannula may be arranged coaxially within the first cannula, preferably along the whole length of the first cannula or preferably at least along at least 90 percent of the length of the first cannula. The first cannula may have a circular or oval cross section having an outer diameter or a maximal outer diameter in the range of 7 mm or 8 mm to 10 mm or 11 mm (Millimeter), i.e. 21 F or 24 F (French) to 31F or 33 F (French). The second cannula may have a circular or oval cross section having a first diameter or a maximal first diameter that is at least 2 mm or at least 3 mm or at least 4 mm smaller than an inner diameter or a maximal inner diameter of the first cannula. The first cannula and the second cannula may have both a circular cross section. Alternatively, the first cannula and the second cannula may have both an oval cross section. The inner cannula may have an outer diameter within the range of 3 mm or 4 mm to 6 mm or 7 mm, i.e. 9 F to 21 F. However, for children cannulas with smaller diameters may be appropriate, for instance 13 F outer diameter of the first cannula.

The outer diameter and/or the inner diameter of the first cannula may be constant along the whole length of the first cannula. Alternatively, the outer diameter and/or the inner diameter of the first cannula may be smaller at a more distal position compared to the diameter at a proximal or more proximal position of the first cannula. Tapering along the whole length or along at least 90 percent of the whole length of the first cannula may be used.

The outer diameter of the second cannula may be constant along the whole length of second cannula. This may make sealing around an insertion opening easier. However, a sealing may be contemplated that is adaptable to different outer diameters of the second cannula depending on the insertion length of a second cannula having a tapered outer form along the whole length or along a part of its length.

The area of the cross section that is used for fluid transport within the first cannula may be equal to the area of the cross section that is used for fluid transport within the second cannula. However, the area of the cross section that is used for fluid transport within the first cannula may be smaller or greater than the area of the cross section that is used for fluid transport within the second cannula. This may depend on the application.

The second cannula may be arranged outside a central position of the first cannula, preferably along the whole overlapping length of the first cannula and the second cannula if the second cannula is inserted completely into the first cannula or preferably at least along at least 90 percent of the overlapping length. Additionally, the first cannula may have a circular or oval cross section having an outer diameter or a maximal outer diameter in the range of 7 mm or 8 mm to 10 mm or 11 mm (Millimeter), i.e. 21 F or 24 F (French) to 31 or 33 F (French). The inner (second) cannula may also have a circular or oval cross section. However, the outer diameter of the inner or second cannula may be within the range of 3 mm or 4 mm to 6 mm or 7 mm, i.e. 9 F to 21 F. Moreover, for children cannulas with smaller diameters may be appropriate, for instance 13 F outer diameter of the first cannula.

The second cannula may be fixed against an axial movement relative to the first cannula after complete insertion of the second cannula. At least one fixation element may be used, preferably outside of the first cannula or of the body. The fixation may be made against an axial movement of the second cannula against the insertion direction of the second cannula and/or against an axial movement of the second cannula in the direction of insertion of the second cannula into the first cannula. It may be easier to have the fixation element outside of the first cannula and also outside of the second cannula for instance with regard to easier operation and/or easier design. However, the at least one fixation element may alternatively also be arranged partially or completely within the outer or first cannula. The fixation element may comprise or consist of a connector for instance. A snap fit or force fit fixation element may be used. It may be possible to loosen or release the fixation element again, for instance in order to remove the second cannula first and thereafter to remove the first cannula out of the body. However, the fixation may be optional because the main part of fixation is done be the diameter variable arrangement or cage arrangement on the distal end of the first cannula and/or of the second cannula.

However, the fixation element could also be within the body, for instance at the distal end of the first cannula or at a position between the proximal end and the distal end of the first cannula. A snap fit element may be used for instance.

The first cannula may be inserted preferably intravascular through the superior vena cava into an interior region of the heart. The second cannula may be inserted through the first cannula into an interior region the heart, preferably to a different interior region of the heart compared to the interior region where the first cannula is positioned, or into the aorta. Thus, the proposed dual lumen cannula may ease surgery within the system of the four heart chambers. A diameter variable arrangement may be mounted on the distal end portion of the first cannula. No membrane may be used there. Alternatively a membrane may be used on this diameter variable arrangement. Examples are described below with regard to FIGS. 2 and 3, i.e. left ventricle assist and bi ventricle assist respectively. FIG. 10 shows a further application example in which a further diameter variable arrangement may be mounted on the first cannula, preferably covered with a membrane that has preferably an opening that faces laterally and or that has preferably an edge that is essentially parallel to cage wires of the diameter variable arrangement. With regard to the application shown in FIG. 10 the diameter variable arrangement on the second or inner cannula may comprise a membrane having an opening that faces distally relative to the longitudinal axis of the second cannula on its distal end.

A diameter variable arrangement, especially a cage arrangement, may be mounted on the distal end portion of the first cannula. The first cannula may not extend into the diameter variable arrangement or does maximally extend into the diameter variable arrangement by at most 10 mm or at most 5 mm or at most 3 mm. This may ease the insertion and/or the bending of the second cannula by an angle of for instance about 90 degrees. A valve or another appropriate element may be arranged on the distal end of the first cannula in order to prevent that blood flows into the first cannula from left atrium to the right atrium through the distal tip of the first cannula or vice versa. Holes may be located in right atrium on a distal end portion of the first cannula.

Only the diameter variable arrangement, especially a cage arrangement, may be arranged in the left atrium and the distal end portion of the first cannula may be arranged completely or essentially within the right atrium.

The first cannula and/or the second cannula may be punctured or pierced through the atrial septum of the heart and the second cannula may be inserted further into the left atrium, the left ventricle and further into the aorta, preferably into the ascending aorta. A diameter variable arrangement may be mounted on the distal end portion of the second cannula. Thus, it is possible to advance the second cannula through three chambers of the heart with small radius of curvature. The second cannula may be bent by more than 150 degrees but for instance less than 180 degrees at one particular position, see also FIGS. 2 and 3. FIG. 10 shows a further application example in which a further diameter variable arrangement may be mounted on the first cannula, preferably covered with a membrane that has preferably an opening that faces laterally and or that has preferably an edge that is essentially parallel to cage wires of the diameter variable arrangement.

Alternatively, the first cannula may be inserted intravascular into an interior region of the heart and the second cannula may be inserted intravascular into the pulmonary artery, into the left pulmonary artery, into the right pulmonary artery and/or into the lung. Examples for lung perfusion and right ventricle assist are shown in FIGS. 10 and 11 respectively. Thus, it may be possible to access the pulmonary artery by the proposed method. Diameter variable arrangements may be used to fix the end of the first cannula and or the end of the second cannula in place.

If access is made to the pulmonary artery, the first cannula may be inserted intravascular through the vena cava, preferably through the superior vena cava up to the right atrium or up to the right ventricle. A first optional diameter variable arrangement may be mounted preferably on the distal end portion of the first cannula. A second diameter variable arrangement may be mounted preferably on the distal end portion of the second cannula. Thus, the second diameter variable arrangement may be inserted through the first diameter variable arrangement. The second diameter variable arrangement may be covered with a membrane that has preferably an edge that is essentially transversally to cage wires of the diameter variable arrangement, i.e. there may be an opening of the membrane that faces distally. Examples are shown in FIGS. 10 and 11. Thus, it is possible to advance the second cannula through two chambers of the heart with a small radius of curvature. The second cannula and/or the first cannula may be bent by at least 150 degrees but for instance less than 180 degrees at a particular position. Other parts of body may also be accessed using the proposed multi lumen cannula, for instance the kidneys, the liver etc.

The first cannula may comprise holes that are placed within the right atrium and within the right ventricle, especially drainage holes. Thus, right heart support is possible in an efficient manner.

There may be a first diameter variable arrangement mounted preferably on a distal end portion of the first cannula, preferably a first cage arrangement. The first diameter variable arrangement may have an expanded state having a maximum first diameter that is greater than the maximum first diameter in a stretched or introducing state, wherein it is in a stretched condition. The maximum diameter in the expanded state may be at least by factor two or at least by factor three greater than in stretched or inserting state. However, the factor may be smaller than 50 or smaller than 10.

A second diameter variable arrangement may be mounted preferably on a distal end portion of the second cannula, preferably a second cage arrangement. The second diameter variable arrangement may have corresponding features as the first diameter variable arrangement.

The length of the first or second diameter variable arrangement may be at most twice or at most threefold its largest diameter if the diameter variable arrangement is in a diameter expanded state. This may be different in comparison to cannulas that are diameter variable along their entire length.

The diameter variable arrangement may be used as fixation means for the distal end of the first cannula or as fixation means for the distal end of the second cannula. Furthermore, the diameter variable arrangement may fulfill further functions. It is for instance possible to attach a membrane at the diameter variable arrangement. The diameter variable arrangement may be inserted into the body in a collapsed form. Within the body, i.e. if it is on place it may be expanded to have the intended technical effects, for instance to define a distance between the sidewall of a blood vasculature and the inlet (outlet) or inlet (outlet) tip of the first cannula or of the second cannula.

The diameter variable arrangement may be a cage arrangement that comprises a plurality of grate elements. A cage may be a comparable simple technical means for spanning a space around the distal end of the second cannula or of the first cannula. Wires may be attached easier to the distal end of first cannula or second cannula compared for instance to interwoven structures. However, stent like structures may also be used for the diameter variable arrangement, especially a braided or interwoven structure. If two diameter variable arrangements are used, one of them may be inserted through the other in order to allow advanced applications in medicine.

There may be 2 to 15 cage wires within a cage arrangement, preferably 3 to 12 cage wires, i.e. 3, 4, 5, 6, 7, 8, 9, 10, 11 or 12 cage wires.

The cage arrangement may be configured in the expanded mode or state as a sphere or as an ellipsoid. The sphere may have the same radius in all directions in the expanded state. The ellipsoid may have three main axis that have different length or at least one of the main axis may be longer than the other two main axis in the expanded state.

The first cage arrangement and/or the second cage arrangement may comprises a plurality of wires. There may be the following portions within the cage arrangement:

a proximal portion, wherein within the proximal portion in the expanded state of the cage arrangement the distance between neighboring wires increases with increasing distance to a mounting portion of the wires,

a distal portion, wherein within the distal portion in the expanded state of the cage arrangement the distance between neighboring wires decreases with increasing distance to the mounting portion of the wires,

an optional transition portion, wherein within the optional transition portion in the expanded state of the cage arrangement the distance between neighboring wires is constant or essentially constant with increasing distance to the mounting portion of the wires.

These characteristics may be valid for all wires within the respective portion. Shape memory material or alloy (SMA) may be used that may vary its shape, especially dependent on temperature. Reference is made to the materials that are listed above for the cage arrangement of the cannula system. Nitinol may be used for instance. Further materials that may be used are super elastic materials, stainless steel wire, cobalt-chrome alloys or cobalt-chromium-nickel-molybdenum-iron alloy.

Thus, wires may be arranged directing or pointing away from each other within a proximal portion of the cage arrangement. Further, wires may be arranged directing or pointing to each other within a proximal portion of the cage arrangement. Within an optional transition or middle portion the wires may be parallel to each other and/or to axial direction. However there may only be essentially two portions. In an embodiment, the wires may be arranged in the middle portion in a different way compared to the arrangement in the proximal portion and in the distal portion of the cage arrangement.

The wires may not cross each other in free portions of the cage arrangement, i.e. in the proximal portion, the distal portion and the optional transient portion, crossing may be possible only within a proximal mounting portion of the wires or at the distal end connecting portion. This may be different form an arrangement of the wires in an interwoven or braided material.

The proximal ends of the cage wires of the first cage arrangement on the first cannula may be wound around a first surface and/or around the distal end portion of the first cannula. Correspondingly, proximal ends of the cage wires of the second cage arrangement on the second cannula may be wound around a surface and/or the distal end portion of the second cannula. Winding and twisting are simple techniques that allow durable connections between the wires and/or the cannula. However, additionally further connecting techniques may be used like glue, welding, soldering etc.

Preferably, the distal ends of the cage wires may be connected with each other, preferably using a connecting element and/or by twisting them. Winding and twisting are simple techniques that allow durable connections between the wires and/or the cannula. However, additionally further connecting techniques may be used like glue, welding, soldering etc. The distal ends may be connected at a position on the longitudinal axis or on the extended longitudinal axis of the first cannula or of the second cannula. This may allow the usage of an introducer for stretching the cage arrangement during introducing. The introducer may be realized without a further increase of the outer diameter compared for instance to the usage of an outer envelope or jacket that would cover the wires and prevent expansion.

In a given axial position, the cage wires may be arranged with regular distances between neighboring wires. However, especially on diameter variable arrangements (cage arrangements) that are mounted within an end portion of the first cannula a part of the wires, e.g. one, two or three wires, may be omitted in order to have a free way for the insertion of the second or second cannula at this position or in this direction. The distance between two neighboring cage wires at a position where at least one cage wire is omitted may be greater than the regular distance, preferably at least twice the regular distance.

Or spoken with other words, the cage wires may be distributed angularly such that, in a given axial position, for two different pairs of neighboring cage wires the wires forming the pair have respectively a first distance relative to each other which is equal for both pairs and that neighboring wires forming yet another pair have a second distance relative to each other that is greater than the first distance, preferably equal to or greater than twice the first distance.

The first cage arrangement and/or the second cage arrangement may comprise a backwards bended portion, preferably following the distal portion of the cage arrangement. Within the backwards bended portion, the wires may change direction and/or neighboring wires may have decreasing distances with decreasing distance to the mounting portions of the wires. Furthermore, the expandable arrangement or the cage arrangement may comprise a cage tip portion following the backwards bended portion, wherein in the cage tip portion the cage wires are connected with each other. The backward bended portion form an atraumatic tip of the cannula.

The first diameter variable arrangement may comprise a first membrane that may be preferably connected to the first diameter variable arrangement. The first membrane may be folded or less stretched in the non-expanded state of the first diameter variable arrangement and the first membrane may be spanned or expanded in the expanded state of the first diameter variable arrangement. Correspondingly, there may be a second membrane that is connected to the second diameter variable arrangement. The second membrane may be folded or less stretched in the non-expanded state of the second diameter variable arrangement. The second membrane may be spanned or expanded in the expanded state of the second diameter variable arrangement.

At least one of the diameter variable arrangements may be covered at least partly by a membrane. The membrane may have an opening that faces distally or an opening that faces laterally or transversally relative to a longitudinal axis of the respective cannula. In both cases, i.e. membrane with opening facing distally or laterally, the membrane may be expanded, e.g. stretched and/or without wrinkles or with less wrinkles, if and when diameter variable arrangement is in diameter expanded state. The membrane may have wrinkles or more wrinkles if and when the diameter variable arrangement is in non-expanded diameter state or in insertion state.

It may be comparable easy to connect membranes to cage wires. Techniques that may be appropriate are sewing, plastic welding, glue and/or dip molding/coating. Pockets may be formed in the membrane in which the wires are inserted. The pockets may be short, i.e. for instance lugs, or they may be long, for instance along the complete length of the main part of the wires or at least 80 percent of the length of the wires, e.g. excluding the attachment portion(s) of the wire. The length may be measured along the cage wires for instance or measured along the longitudinal axis of the first cannula or second cannula, preferably on its distal end. It is possible to use membranes if three or more cage wires are used, for instance 4 or more than 4 cage wires.

The membrane may be a thin sheet of material, having for instance a thickness of less than 0.5 millimeters, less than 100 micrometer or less than 50 micrometer. However, the membrane may be thicker than 10 micrometer in order to provide appropriate strength. Polytetrafluoroethylene (PTFE) may be an appropriate material for the membrane, preferably if it is manufactured by electro spinning, for instance within an electrical field. However, other materials may also be used.

If two membranes on both diameter variable arrangements are used, the second membrane may be inserted through the first membrane. This and the insertion of a cage through a cage allows advanced applications in medicine.

In the expanded state of the first diameter variable arrangement an edge of the first membrane may define an opening that faces distally relative to the first cannula. In the expanded state of the second diameter variable arrangement an edge of the second membrane may define an opening that faces distally relative to the second cannula. The edge of membrane may delimit the edge of the opening along the entire circumference of the opening. Thus, the membrane may extend circumferential around the longitudinal axis of the first cannula or the second cannula by at least 360 degrees or by 360 degrees. The membrane having the distal opening may extend from a proximal end of the diameter variable arrangement at most three quarters or at most half way or at most one quarter to a distal end of the diameter variable arrangement.

A membrane having a distally facing opening may have, especially in the expanded state, the following relations relative to the portions of the cage arrangement that are mentioned above:

the proximal portion or parts thereof and/or the optional transition portion or parts thereof may be covered by the membrane, and

the distal portion and/or the optional transition portion may not be covered by the membrane.

Alternatively, in the expanded state of the first diameter variable arrangement, an edge of the first membrane may define an opening that faces laterally relative to the first cannula. Correspondingly, in the expanded state of the second diameter variable arrangement an edge of the second membrane may define an opening that faces laterally relative to the second cannula. Again, an edge of the membrane may delimit the edge of the opening along the entire circumference of the opening. The membrane having an opening facing laterally may extend circumferential around the extension of the longitudinal axis of the first cannula or the second cannula by less than 270 degrees or by less than 200 degrees and may extend from a proximal end of the diameter variable arrangement to a distal end of the diameter variable arrangement or along at least 80 percent of this distance. The membrane may cover the cage arrangement from 0 degree to 180 degrees or from 0 degree to 210 degrees or from 0 degree to 240 degrees.

A membrane having a laterally facing opening may have, especially in the expanded state, the following relations relative to the portions of the cage arrangement that are mentioned above:

the proximal portion, the optional transition portion, and the distal portion may be covered by the membrane on a first side of the cage arrangement, and

the proximal portion, the optional transition portion and the distal portion may not be covered by the membrane on a second side of the cage arrangement that is preferably opposite to the first side of the cage arrangement.

In both cases, i.e. membrane comprising a distally facing opening and membrane comprising a laterally facing opening the volume that is defined by the membrane may be an extension of the inner lumen of the lumen portion, i.e. it may extend the lumen for a fluid that flows out of the cannula or it may narrow the lumen for a fluid that flows into the cannula, e.g. same as a funnel.

The first cannula and the second cannula may form a first multi lumen cannula system and a second multi lumen cannula system may be used at the same time within the same body, preferably inserted in the same organ as the first multi lumen cannula system. The usage of two multi lumen cannula systems opens advanced applications in medicine.

The first cannula of the second multi lumen cannula system may be inserted into the body first. After insertion of the first cannula of the second multi lumen cannula system, a second cannula of the second multi lumen cannula system may be inserted axially within the first cannula of the second multi lumen cannula system, preferably until it extends axially over the first cannula of the second multi lumen cannula. Therefore, it is also possible to insert the second multi lumen cannula system smoothly into the body, preferably with reducing the risk of injuries by inserting two cannulas stepwise thereby reducing friction on body tissue and overall bending stiffness. Thus, the same features as described above for the lumens and/or fluids that are valid for the cannulas of the first multi lumen cannula system may be valid for cannulas of the second multi lumen cannula system as well. Two or more than two dual or multi lumen cannula systems may be used at one time. There may be more than two cannulas within a multi lumen cannula system.

According to FIG. 10, the following method for lung perfusion, i.e. right heart support, and left heart support is disclosed:

wherein the distal end of the first cannula of the first multi lumen cannula system is placed within left atrium,

wherein the distal end of the first multi lumen cannula system is placed within the aorta, preferably within the ascending aorta,

wherein preferably a first diameter variable arrangement is mounted on the distal end portion of the first cannula of the first multi lumen cannula system, preferably covered with a membrane that has an opening that faces laterally and/or that is essentially parallel to two of the cage wires of the diameter variable arrangement,

and/or wherein preferably a second diameter variable arrangement is mounted on the distal end portion of the second cannula of the first multi lumen cannula system, preferably covered with a membrane that preferably has an opening facing distally and/or that is essentially transversally to the cage wires of the diameter variable arrangement,

wherein the distal end of the first cannula of the second multi lumen cannula system is placed within the right atrium or the right ventricle, and

wherein the distal end of the second multi lumen cannula system is placed within the pulmonary artery, preferably within the left pulmonary artery or within the right pulmonary artery,

and/or wherein preferably a third diameter variable arrangement is mounted on the distal end portion of the second cannula of the second multi lumen cannula system, preferably covered with a membrane that has preferably an opening facing distally and/or that is essentially transversally to the cage wires of the diameter variable arrangement.

According to FIG. 10, the following flows may be established for retrograde (opposite direction to natural blood flow direction in lung vessels) lung perfusion:

first flow from distal end of second cannula of second multi lumen cannula system through second cannula of second multi lumen cannula out of an outlet port of second cannula of second multi lumen cannula system through a first circuitry, preferably comprising a pump, into an inlet port of first cannula of first multi lumen cannula system through first cannula of first multi lumen cannula system out of distal end of first cannula of first multi lumen cannula system,

second flow from distal end of first cannula of second multi lumen cannula system through first cannula of second multi lumen cannula system through a first circuitry, preferably comprising a group of a pump and an oxygenator device, into an inlet port of second cannula of first multi lumen cannula system through second cannula of first multi lumen cannula system out of distal end of second cannula of first multi lumen cannula system.

According to FIG. 10, the following flows may be established for antegrade (in direction that is equal to natural blood flow direction in lung vessels) lung perfusion:

first flow from distal end of first cannula of first multi lumen cannula system through first cannula of first multi lumen cannula system out of an outlet port of first cannula of first multi lumen cannula system through an first circuitry, preferably comprising a pump, into an inlet port of second cannula of second multi lumen cannula system through second cannula of second multi lumen cannula system out of distal end of second cannula of second multi lumen cannula system,

second flow from distal end of first cannula of second multi lumen cannula system through first cannula of second multi lumen cannula system through an first circuitry, preferably comprising a group of a pump and an oxygenator device, into an inlet port of second cannula of first multi lumen cannula system through second cannula of first multi lumen cannula system out of distal end of second cannula of first multi lumen cannula system.

At least one sealing element may be arranged within an opening through which the second cannula of the first multi lumen cannula system may be inserted into the first cannula, preferably a retaining ring, a sealing ring, a gasket a multi-flap valve or another self-sealing element.

A closure element that prevents the passage of fluid through the distal end of the first cannula into the first lumen of the first cannula or vice versa may be used. The closure element may be arranged on a distal end portion of the first cannula. The closure element may allow the passage of the second cannula and/or of an introducer. The closure element may comprise at least one sealing membrane, preferably at least one membrane comprising an aperture and/or at least one self-sealing flap. An example is for instance a sealing element that has two flexible membranes each comprising an aperture and both apertures have a lateral offset relative to each other. If an introducer or the second cannula stretches the membranes the offset between the apertures may become smaller and finally both apertures may be passed by the introducer or the second cannula. Alternatively, the membrane may not have an aperture but is pierced by the second cannula or by an auxiliary tool. Moreover, a multi-flap valve may also be an appropriate element.

Thus it is possible to place a cage arrangement for instance within the left atrium. The first cannula may not extend or may extend maximal 5 mm or maximal 3 mm into the cage arrangement and therewith into the left atrium. The technical effect is that no blood or only less blood is allowed to flow from left atrium into the first cannula. The first cannula may comprise holes at a distal portion which are placed within the right atrium. These holes may be especially drainage holes. A further technical effect is that the second cannula may forwarded in an easy way further through the distal tip of the first cannula and also through the cage arrangement on the distal tip of the first cannula for instance into the left ventricle and even further into the aorta, especially into the ascending aorta.

The first fluid and/or the second fluid may be injected into the body and/or taken out of the body in a pulsed manner using a pump or in a continuous flow using also an appropriate pump, especially an external pump. A pulsed flow is the natural mode of transport of body fluids and may result in less damage to the body and/or to specific organs.

The first cannula or the second cannula may be pre-bended or both cannulas may be pre bended by an angle within the range of 60 degrees to 175 degrees or in the range of 75 degrees to 145 degrees, preferably in order to ease an insertion through the septum of the heart, preferably through the atrial septum. The mix or combination of the pre-bending and the diameter variable arrangement and/or of using at least one membrane opens new application in medicine, especially in heart surgery or treatment.

The distal end of the first cannula may be placed within left atrium. The distal end of the second cannula may be placed within the aorta, preferably within the ascending aorta. Preferably, a first diameter variable arrangement may be mounted on the distal end portion of the first cannula, preferably covered with a membrane that has an opening that faces laterally and/or that is essentially parallel to two of the cage wires of the diameter variable arrangement. Preferably, a second diameter variable arrangement may be mounted on the distal end portion of the second cannula, preferably covered with a membrane that preferably has an opening facing distally and/or that is essentially transversally to the cage wires of the diameter variable arrangement. A further cannula may be inserted into the coronary sinus vein and through a puncture between the coronary sinus vein and the left atrium up to the left atrium. The further cannula may comprise a (third) diameter variable arrangement, e.g. a cage on its distal end. Membranes may be used or may not be used on the (third) diameter variable arrangement on the further cannula. The technical effect that may be reached may be a very high pumping volume per minute out of the left atrium. Nevertheless, it is possible to flow at the same time blood through the dual lumen cannula, for instance through the inner cannula of the dual or multi lumen cannula, into the aorta. Thus, an excellent blood circulating system is provided.

A percutaneous biventricular assist arrangement/device (pBiVAD) may be realized which may be configured to drain blood from the left atrium through at least one opening of the second cannula and to drain blood from the right atrium through at least one opening of the first cannula. The second cannula may be inserted into the first cannula within the body, in order to reduce trauma and mechanical stress to the blood vessels. There may be a combination of the pBiVAD® with an oxygenation, for instance with an ECMO (extracorporeal membrane oxygenation). Alternatively, no oxygenation may be used. A third cannula, preferably a single lumen cannula, may be used to pump blood into the aorta, e.g. in the ascending aorta or in the descending aorta. Both cannulas, i.e. the dual lumen cannula and the single lumen cannula may be inserted through the artrial septum of the heart. Furthermore, both cannulas may be inserted through the jugular veins, e.g. each cannula through a respective one of the jugular veins. There may be a complete oxygenation of all of the drained blood. Alternatively, only blood which is drained from right atrium may be oxygenated but not blood drained from the left atrium which may be oxygenated by the lungs of a patient.

The cannula system or its embodiments may be used to perform the method or its embodiments. Thus, corresponding technical effects apply. Vice versa, the cannula and its embodiments may have features which are mentioned only for the method. These features may also be used for the cannula and its embodiments and may have the same or similar technical effects.

Definitions

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, Leuenberger, H. G. W, Nagel, B. and Kölbl, H. eds. (1995), Helvetica Chimica Acta, CH-4010 Basel, Switzerland).

The term “haemoperfusion/hemoperfusion”, as used herein, refers to a method of filtering blood extracorporeally (that is, outside the body) to remove one or more toxins. As with other extracorporeal methods, such as hemodialysis (HD), hemofiltration (HF), and hemodiafiltration (HDF), the blood travels from the patient into a machine, gets filtered, and then travels back into the patient, typically by venovenous access (out of a vein and back into a vein). During the extracorporeal process, inflammatory and other harmful molecules are removed from the blood of the organism of a patient. Adsorbers (filters), in particular macroporous resin beads, are preferably used, which allow an effective blood purification. These adsorbers in turn may be combined with other therapy components, which regulate the metabolism and strengthen the immune system. In this way, drug strategies can take effect more effectively. Alternatively and/or additionally absorber techniques may be used. However, the filtering may alternatively concern a fluid flow that does not contain blood or that does not contain blood as the main part, i.e. it contains blood or blood components only by 50 percent per volume or less than 50 volume percent.

The term “hyperoxygenated haemoperfusion/hemoperfusion”, as used herein, refers to the haemoperfusion/hemoperfusion as described above but extended by an oxygenator that generates an oxygen level in the blood or in the fluid flow that is used for treatment that is higher than a normal oxygen level within blood, for instance if the body rests. This the oxygen content of the blood may be increased, for instance at least by 5 percent or at least by 10 percent. The oxygen content of body tissue may be increased even more, for instance by more than 10 percent or more than 50 percent compared for instance to a normal oxygen level, for instance if the body rests. Hyperoxygenation may have a positive effect for the uptake of medicaments and/or treatment substances by the body or more specific by the organ that is under treatment.

The term “hypooxygenated haemoperfusion/hemoperfusion”, as used herein, refers to the haemoperfusion/hemoperfusion as described above but extended by an oxygenator that generates an oxygen level that is lower than the normal oxygen level within blood, for instance if the body rests. This may reduce the oxygen content of the blood and/or of body tissue, for instance by more than 10 percent or by more than 50 percent. Hypooxygenation may have a positive effect for the uptake of medicaments and/or treatment substances by the body or more specific by the organ that is under treatment.

Furthermore, there may be medicaments and/or treatment substances that react with oxygen. This reaction may be detrimental for the therapeutic effect and it may be advantageous to prevent or reduce the reaction as far as possible.

The term “disease”, as used herein, refers to an abnormal condition that affects the body of an individual. A disease is often construed as a medical condition associated with specific symptoms and signs. A disease may be caused by factors originally from an external source, such as infectious disease, or it may be caused by internal dysfunctions, such as autoimmune disease. In humans, “disease” is often used more broadly to refer to any condition that causes pain, dysfunction, distress, social problems, or death to the individual afflicted, or similar problems for those in contact with the individual. In this broader sense, it sometimes includes injuries, disabilities, disorders, syndromes, infectious, isolated symptoms, deviant behaviors, and atypical variations of structure and function, while in other contexts and for other purposes these may be considered distinguishable categories. Diseases usually affect individuals not only physically, but also emotionally, as contracting and living with many diseases can alter one's perspective on life, and one's personality. In the context of the present invention, the disease may be selected from the group consisting of cancer and infectious disease.

The terms “cancer disease” or “cancer”, as used herein, refer to or describe the physiological condition in an individual that is typically characterized by unregulated cell growth. Examples of cancers include, but are not limited to, carcinoma, lymphoma, blastoma and sarcoma. More particularly, examples of such cancers include lung cancer, liver cancer or cancer of other organs.

The terms “individual” and “subject” can be used interchangeable herein. The individual or subject may be any mammal, including both a human and another mammal, e.g. an animal. Human individuals or subjects are particularly preferred. The individual may be a patient.

The term “patient”, as used herein, refers to any subject suffering from a disease, in particular suffering from cancer, an autoimmune disease, and/or infectious disease. The patient may be treated and/or the response to said treatment may be evaluated. The patient may be any mammal, including both a human and another mammal, e.g. an animal. Human subjects as patients are particularly preferred.

The term “treatment”, in particular “therapeutic treatment”, as used herein, refers to any therapy which improves the health status and/or prolongs (increases) the lifespan of a patient. Said therapy may eliminate the disease in a patient, arrest or slow the development of a disease in a patient, inhibit or slow the development of a disease in a patient, decrease the frequency or severity of symptoms in a patient, and/or decrease the recurrence in a patient who currently has or who previously has had a disease.

A drug used in chemotherapy is a chemotherapeutic agent. The term “chemotherapeutic agent”, as used herein, refers to a compound that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. The chemotherapeutic agent is preferably selected from the group consisting of alkylating agents, anthracyclines, cytoskeletal disruptors (taxanes), epothilones, histone deacetylase inhibitors, inhibitors of topoisomerase I, inhibitors of topoisomerase II, kinase inhibitors, nucleotide analogs and precursor analogs, peptide antibiotics, platinium-based agents, retinoids and vinca alkaloids and its derivatives. Liquid drugs may be used.

However, usage of small balls or beads or nano particles or micro particles may be advantageous to deliver the medicament and/or the therapeutic substance, for instance usage of nanoballs. Liposomes may be used as nano particles or as micro particles.

The term “radiation therapy (also called radiotherapy)”, as used herein, refers to a cancer treatment that uses high doses of radiation to kill cancer cells and shrink tumors. At low doses, radiation is used in X-rays to see inside the body. At high doses, radiation therapy kills cancer cells or slows their growth by damaging their DNA. Cancer cells whose DNA is damaged beyond repair stop dividing or die. When the damaged cells die, they are broken down and removed from the body. Radiation therapy may not kill cancer cells right away. It may take days or weeks of treatment before DNA may be damaged enough for cancer cells to die. Then, cancer cells may keep dying for weeks or months after radiation therapy ends. Classical radiation by using ionizing radiation (high energy protons, electrons, neutrons, photons, particles) or by electronic X-ray devices may be used. Alternatively, radioactive substances may be brought into contact with the body, specifically with the treated organ or with a part of treated organ.

However, usage of small balls or beads or nano particles or micro particles may be advantageous to bring radioactive substances into the body, for instance usage of nanoballs.

The term “extracorporeal blood”, as used herein, refers to blood removed/isolated from an individual's blood circulation.

The term “extracorporeal circuit”, as used herein, refers to a procedure in which blood is taken from an individual's circulation to have a process applied to it before it is returned to the circulation. All of the system carrying the blood outside the body is termed the extracorporeal circuit.

The term “systemic administration”, a used herein, refers to the administration of the therapeutic agent, e.g. chemotherapeutic agent, such that said agent becomes widely distributed in the body of a patient in significant amounts and develops a biological effect. Typical systemic routes of administration include administration by introducing the therapeutic agent, e.g. chemotherapeutic agent, directly into the vascular system or oral, pulmonary, or intramuscular administration wherein the therapeutic agent, e.g. the chemotherapeutic agent, enters the vascular system and is carried to one or more desired site(s) of action via the blood. The systemic administration may be by parenteral administration. However, the proposed cannulas and methods may be especially advantageous for more local treatment of organs or of parts of organs.

The term “parenteral administration”, as used herein, refers to the administration of the therapeutic agent, e.g. chemotherapeutic agent, such that said compound does not pass the intestine. The term “parenteral administration” includes intravascular administration, intravenous administration, subcutaneous administration, intradermal administration, or intraarterial administration, but is not limited thereto.

It is also preferred that the extracorporeal blood is oxygenated blood. Preferably, the extracorporeal blood has been oxygenated by external means, e.g. using an oxygenator. The oxygenator enhances oxygen within the blood. Alternatively, the oxygen content of blood may be reduced below a normal level. However, normal oxygen levels within blood may be used as well.

It is further preferred that the blood is purified blood. Extracorporeal blood purification (EBP) is a treatment in which a patient's/donor's blood is passed through a device (e.g. membrane, sorbent) in which solute (e.g. waste products, toxins) and possibly also water are removed. When fluid is removed, replacement fluid is usually added. It is preferred that purified blood does not comprise inflammatory, toxic molecules and/or other harmful molecules anymore or comprises a reduced amount of said molecules compared to unpurified blood. Extracorporeal therapies designed to remove or filter substances from the circulation in order to purify blood include hemodialysis, hemofiltration, hemoadsorption, plasma filtration, cell-based therapies and combinations of any of the above. Preferably, the purified blood is filtered blood. More preferably, the purified blood is dialyzed blood. Blood dialysis removes waste, salt, toxins, extra water to prevent them from building up in the body, keeps a safe level of certain chemicals in the blood such as potassium, sodium, and bicarbonate, and helps to control blood pressure. It is particularly preferred that the blood, e.g. the purified, filtered, or dialyzed blood, is free of inflammatory or other harmful molecules.

It is further preferred that the cancer is selected from the group consisting of, lung cancer, urothelial cancer, bladder cancer, liver cancer, kidney cancer/renal cancer, stomach cancer.

It is further preferred that the chemotherapeutic agent is selected from the group consisting of alkylating agents, anthracyclines, cytoskeletal disruptors (taxanes), epothilones, histone deacetylase inhibitors, inhibitors of topoisomerase I, inhibitors of topoisomerase II, kinase inhibitors, nucleotide analogs and precursor analogs, peptide antibiotics, platinium-based agents, retinoids vinca alkaloids and its derivatives.

Systemic routes of administration may include administration by introducing the chemotherapeutic agent directly into the vascular system or pulmonary wherein the chemotherapeutic agent enters the vascular system and is carried to one or more desired site(s) of action. This is described in more detail below.

In particular, the chemotherapeutic agent may be suitable to be administered topically, intravenously, intraarterially, intrapleurally, by inhalation, via a catheter. The dose which can be administered to a patient (“a therapeutically effective amount” or simply “an effective amount”) should be sufficient to effect a beneficial therapeutic response in the patient over time. The dose will be determined by the efficacy of the particular chemotherapeutic agent employed and the condition of the patient, as well as the body weight or surface area of the patient to be treated. The size of the dose also will be determined by the existence, nature, and extent of any adverse side-effects that accompany the administration of the chemotherapeutic agent in a particular patient. The proposed invention may reduce adverse side-effects tremendously as the chemotherapeutic agent may be applied only locally and isolated from other body fluid circuits, especially form the blood circulation circuit of the body.

Due to the local treatment, the chemotherapeutic agent may be administered in an amount that is higher or even much higher than the amounts that used in systemic administration.

The proposed method and its embodiments may not be used for treatment of the human or animal body by surgery or therapy and may not be a diagnostic method practiced on the human or animal body. Alternatively, the proposed method and its embodiments may be used for treatment of the human or animal body by surgery or therapy and may be a diagnostic method practiced on the human or animal body.

The making and using of the presently preferred embodiments are discussed in detail below. It should be appreciated, however, that the present disclosure provides many applicable concepts that can be embodied in a wide variety of specific contexts. The specific embodiments discussed are merely illustrative of specific ways to make and use the disclosed concepts, and do not limit the scope of the claims.

Moreover, same reference signs refer to the same technical features if not stated otherwise. As far as “may” is used in this application it means the possibility of doing so as well as the actual technical implementation. The present concepts of the present disclosure will be described with respect to preferred embodiments below in a more specific context namely heart and lung surgery. The disclosed concepts may also be applied, however, to other situations and/or arrangements in heart and lung surgery as well, especially to surgery of other organs.

The foregoing has outlined rather broadly the features and technical advantages of embodiments of the present disclosure. Additional features and advantages of embodiments of the present disclosure will be described hereinafter, e.g. of the subject-matter of dependent claims. It should be appreciated by those skilled in the art that the conception and specific embodiments disclosed may be readily utilized as a basis for modifying or designing other structures or processes for realizing concepts which have the same or similar purposes as the concepts specifically discussed herein. It should also be recognized by those skilled in the art that equivalent constructions do not depart from the spirit and scope of the disclosure, such as defined in the appended claims.

For a more complete understanding of the presently disclosed concepts and the advantages thereof, reference is now made to the following description in conjunction with the accompanying drawings. The drawings are not drawn to scale. In the drawings the following is shown in:

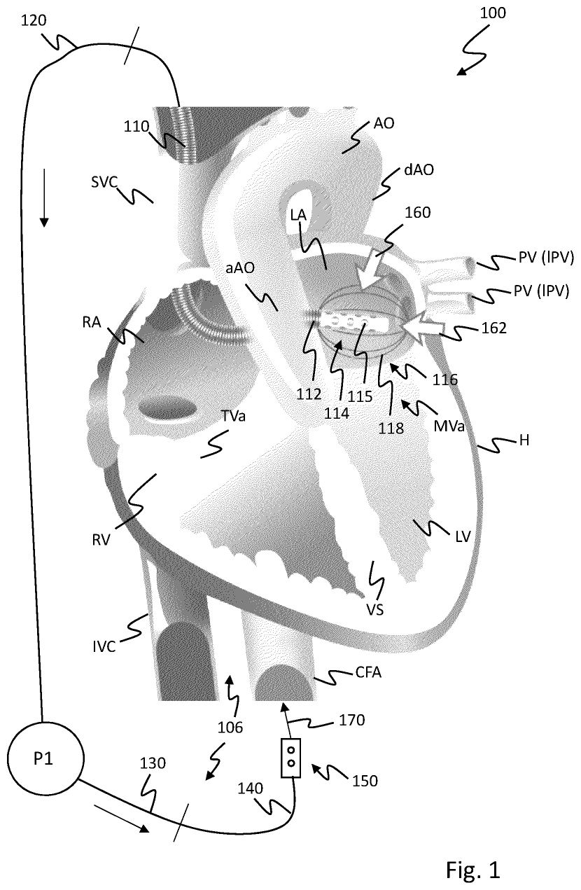

FIG. 1 an extra corporeal blood flow circuitry comprising a two single lumen cannulas,

FIG. 2 an extra corporeal blood flow circuitry comprising a dual lumen cannula,

FIG. 3 an extra corporeal blood flow circuitry comprising a dual lumen cannula, a blood pump and oxygenator,

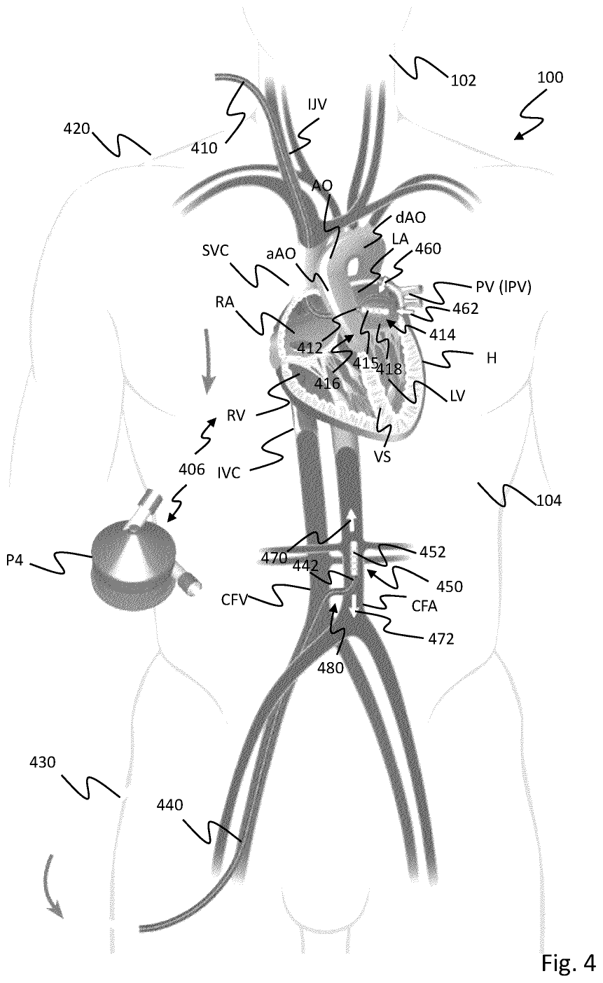

FIG. 4 a transcaval extra corporeal blood flow circuitry comprising two single lumen cannulas,

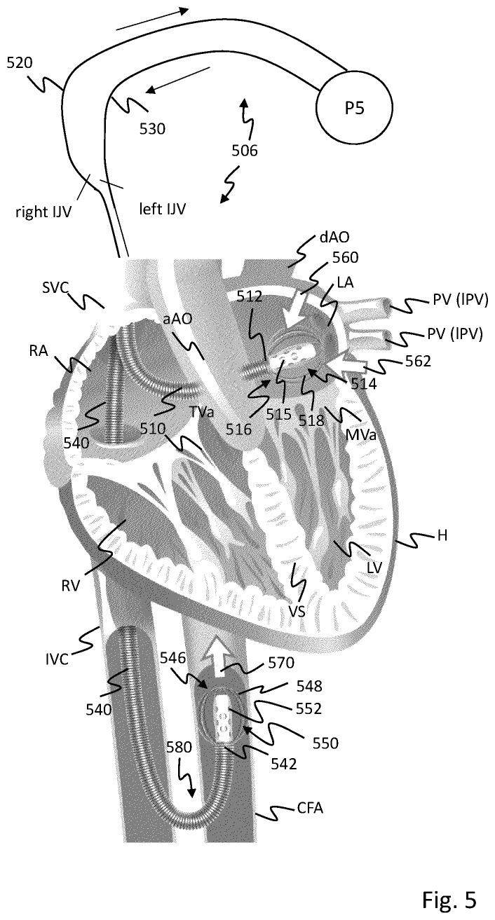

FIG. 5 a transcaval extra corporeal blood flow circuitry comprising a two single lumen cannulas both inserted through jugular veins,

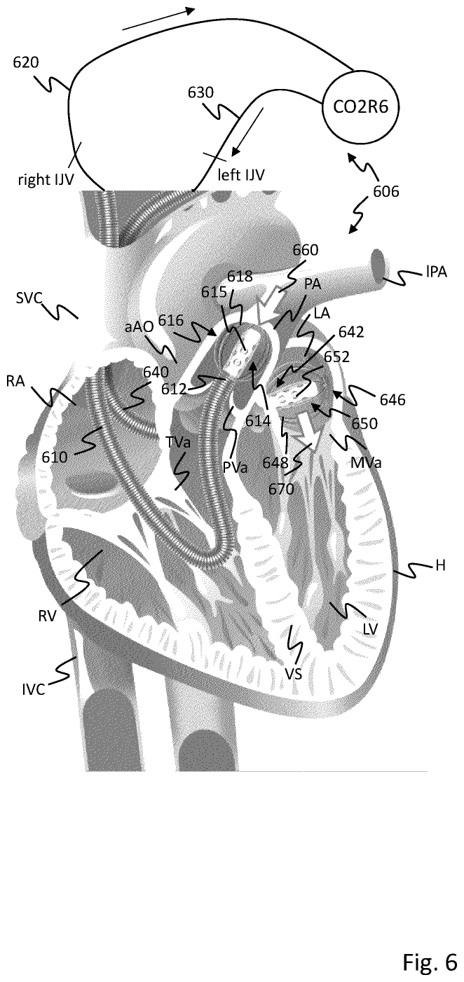

FIG. 6 an extra corporeal lung assist blood flow circuitry without pump comprising two single lumen cannulas and a carbon dioxide removal device,

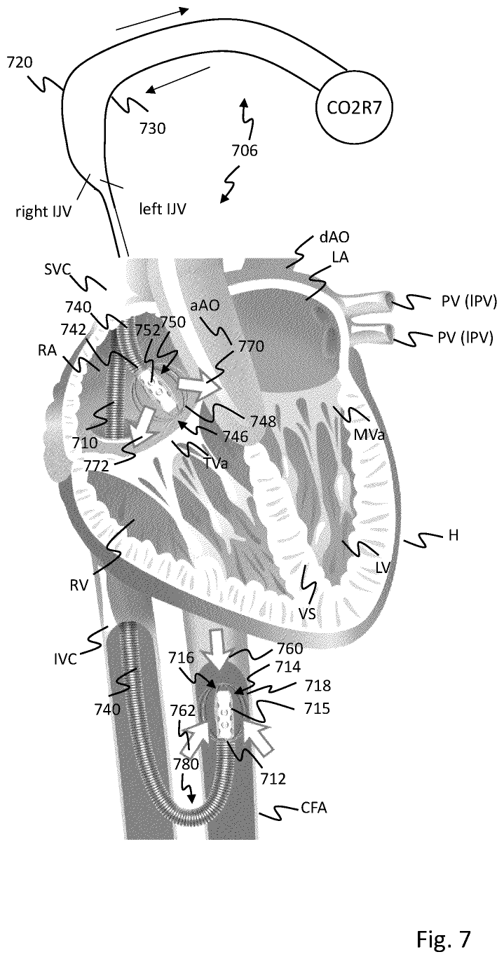

FIG. 7 a transcaval extra corporeal lung assist blood flow circuitry without pump comprising two single lumen cannulas and a carbon dioxide removal device,

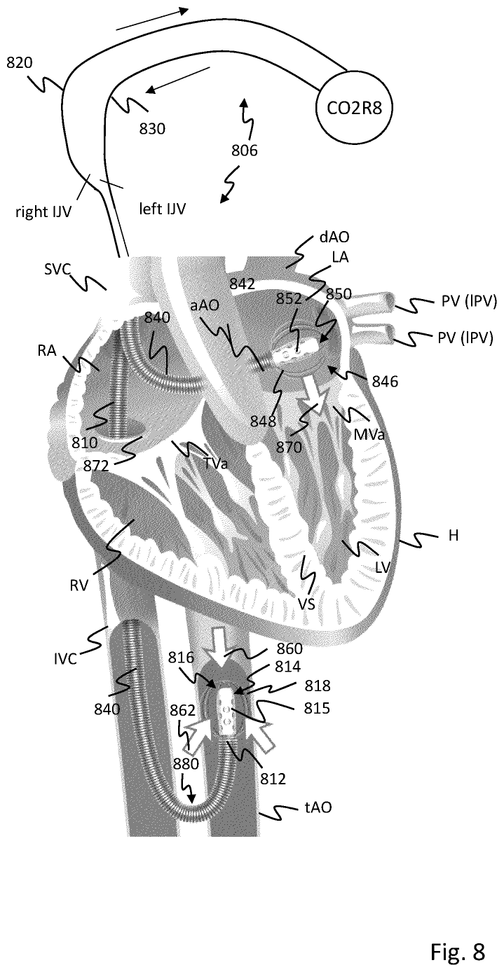

FIG. 8 a transcaval transseptal extra corporeal lung assist blood flow circuitry without pump comprising two single lumen cannulas and a carbon dioxide removal device,

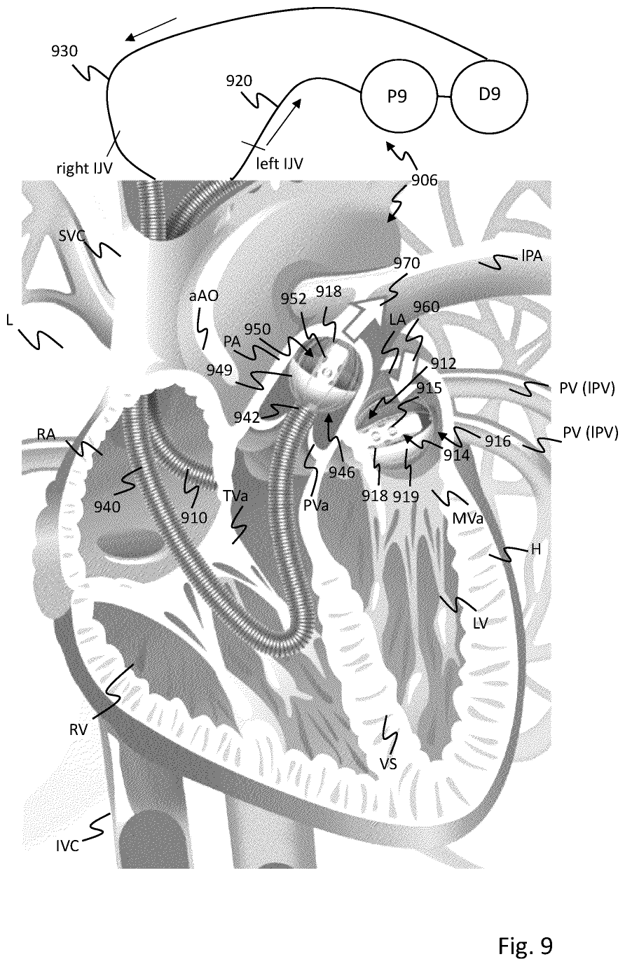

FIG. 9 an extra corporeal circular lung perfusion blood flow circuitry comprising two single lumen cannulas, a pump and a further device,

FIG. 10 an extra corporeal retrograde lung perfusion circular blood flow circuitry comprising two dual lumen cannulas, an alternative embodiment with antegrade lung perfusion, an alternative embodiment with lobe dedicated lung perfusion and an embodiment for right ventricle assist,

FIG. 11 a right ventricle assist circuitry with one inlet stage or with multi inlet stages,

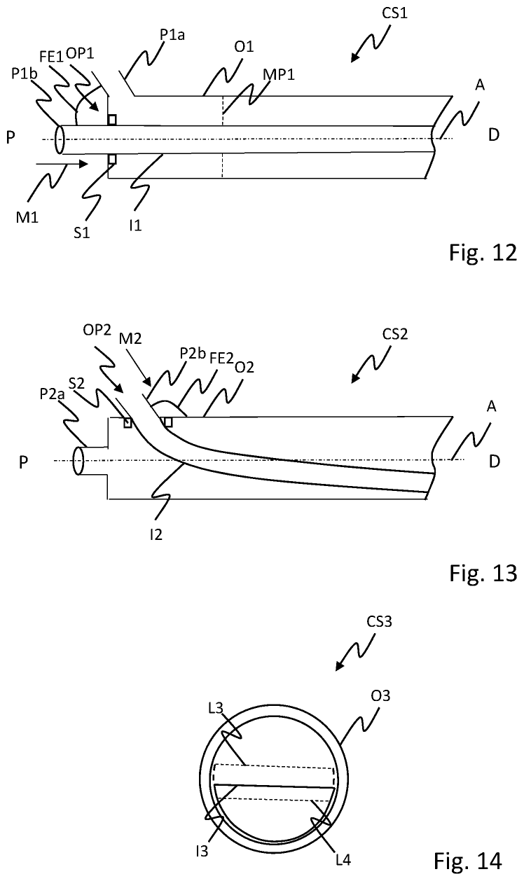

FIG. 12 a cannula system having cannulas that are arranged coaxially,

FIG. 13 a cannula system having an inner (second) cannula that is arranged loosely within an outer (first) cannula,

FIG. 14 a cross section of another cannula system,

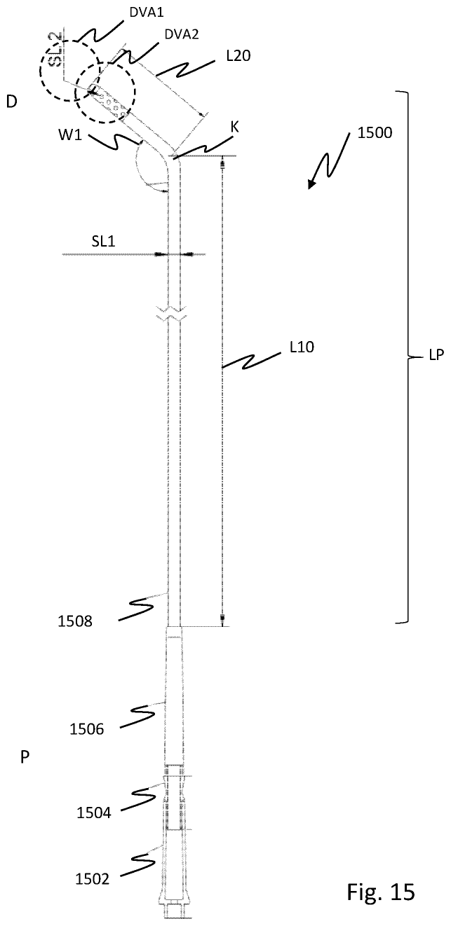

FIG. 15 an embodiment of a dual lumen system comprising at least one pre bended cannula,

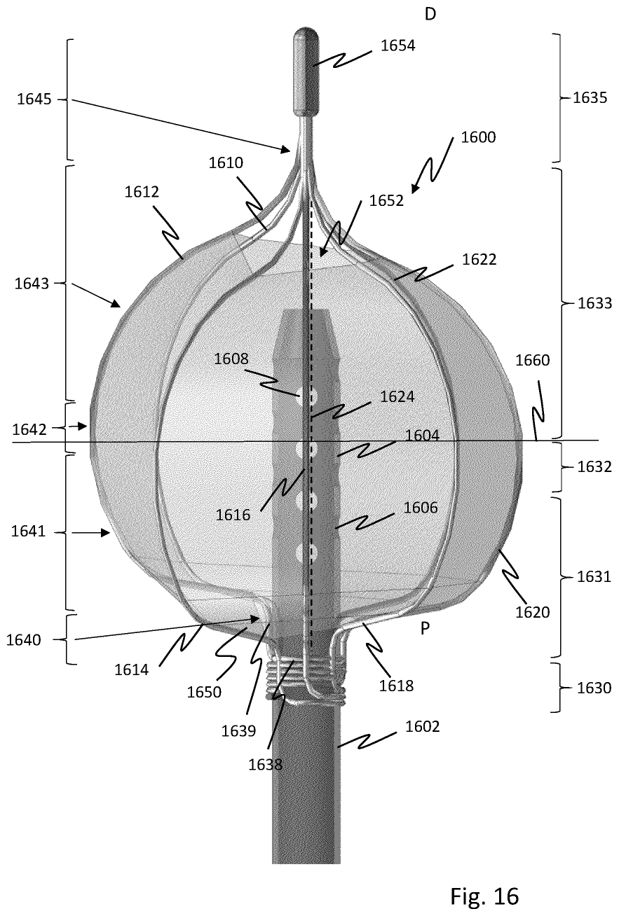

FIG. 16 a cage arrangement comprising a membrane having an opening that faces distally,

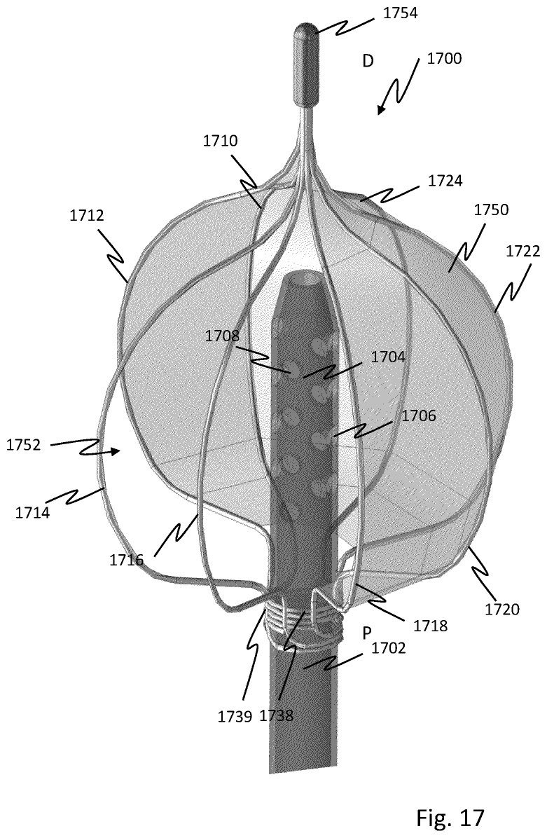

FIG. 17 a cage arrangement comprising a membrane having an opening that faces laterally,

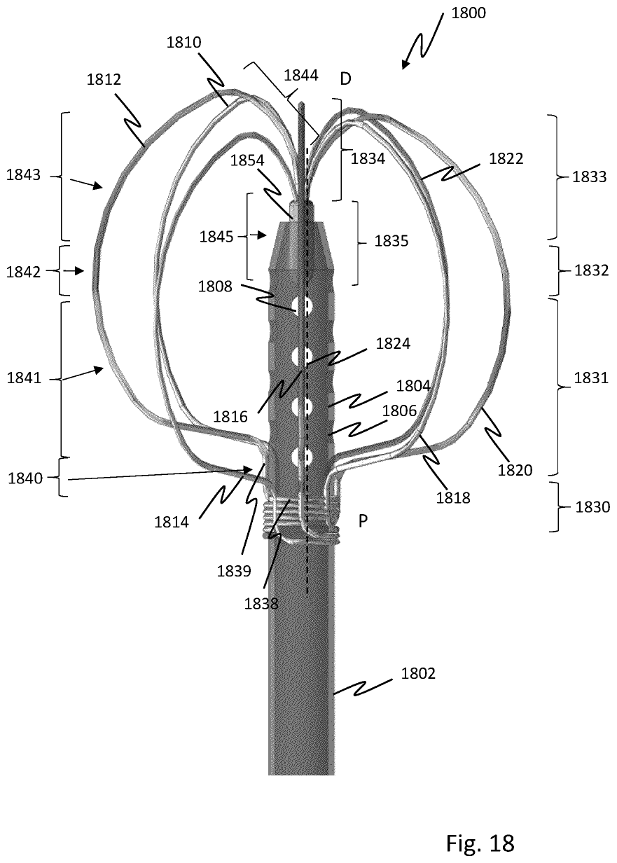

FIG. 18 a cage arrangement comprising a portion that is bended backwards,

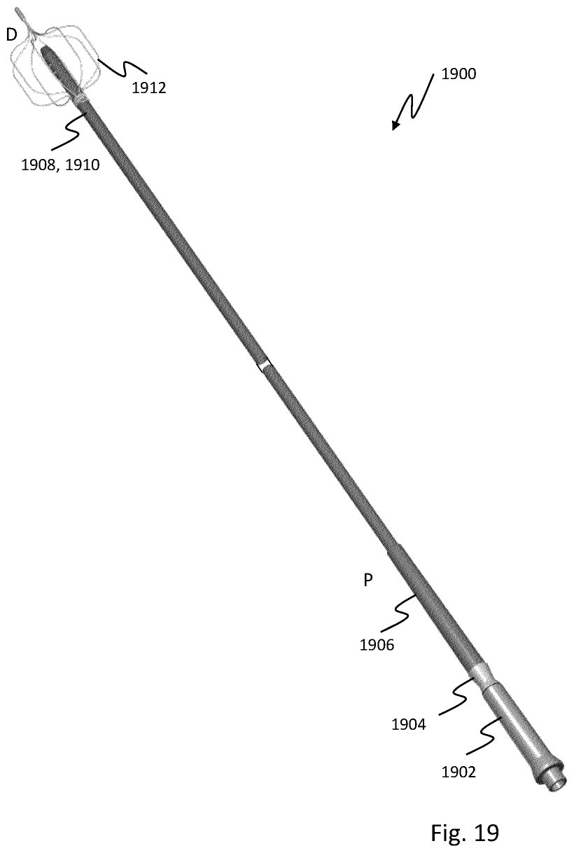

FIG. 19 a cannula comprising a cage arrangement having wires that are arranged in parallel with regard to each other,

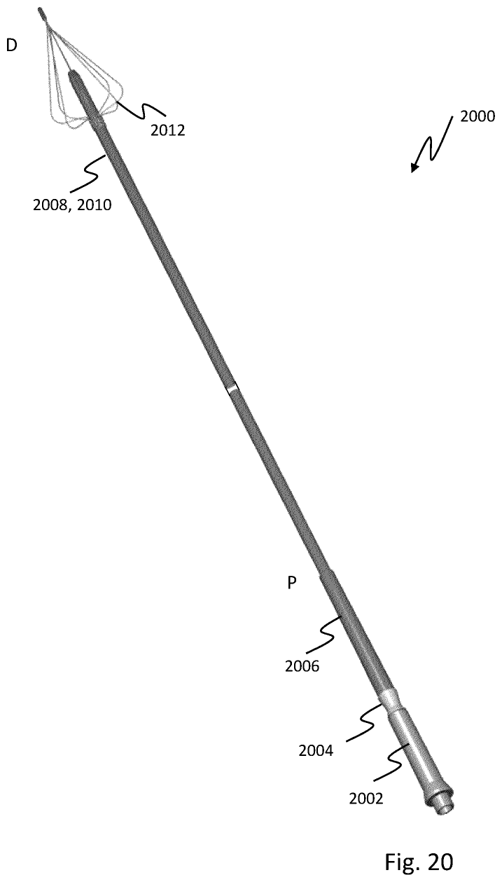

FIG. 20 a cannula comprising a cage arrangement having a cone like shape,

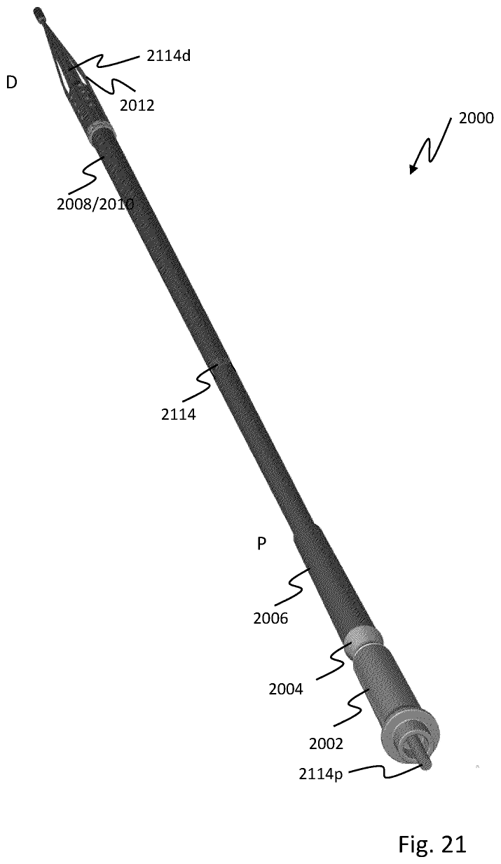

FIG. 21 the cannula of FIG. 20 in a state in which an introducer stretches the cage arrangement for introducing the cannula into a body,

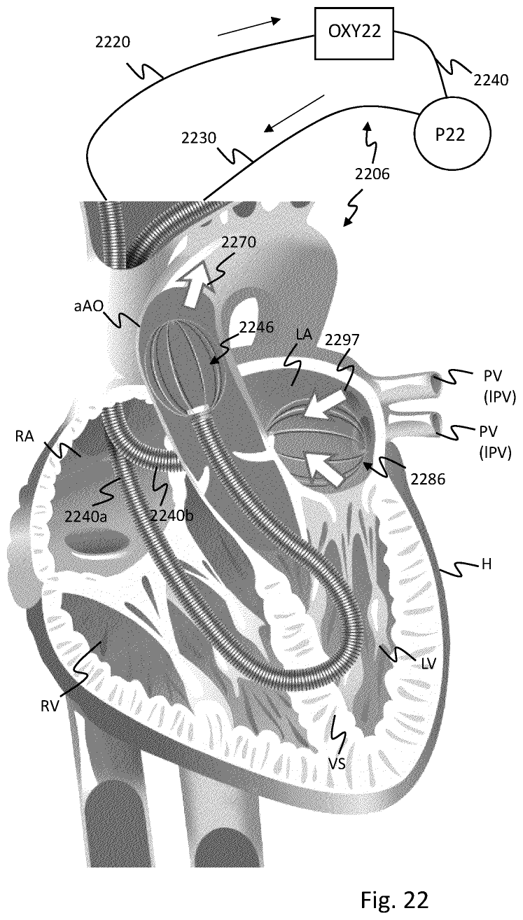

FIG. 22 an alternative embodiment wherein a cannula is pierced or punctured through a ventricle septum,

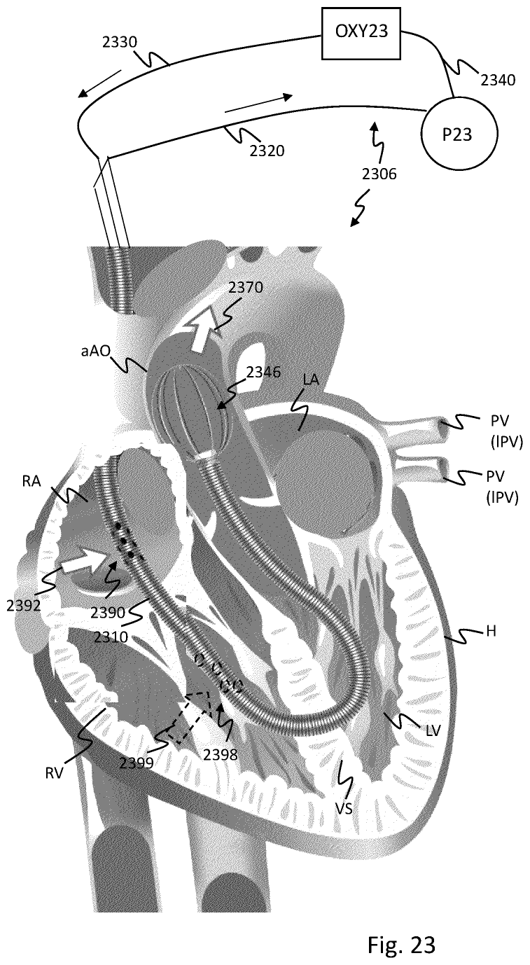

FIG. 23 a further alternative embodiment wherein a cannula is pierced or punctured through a ventricle septum,

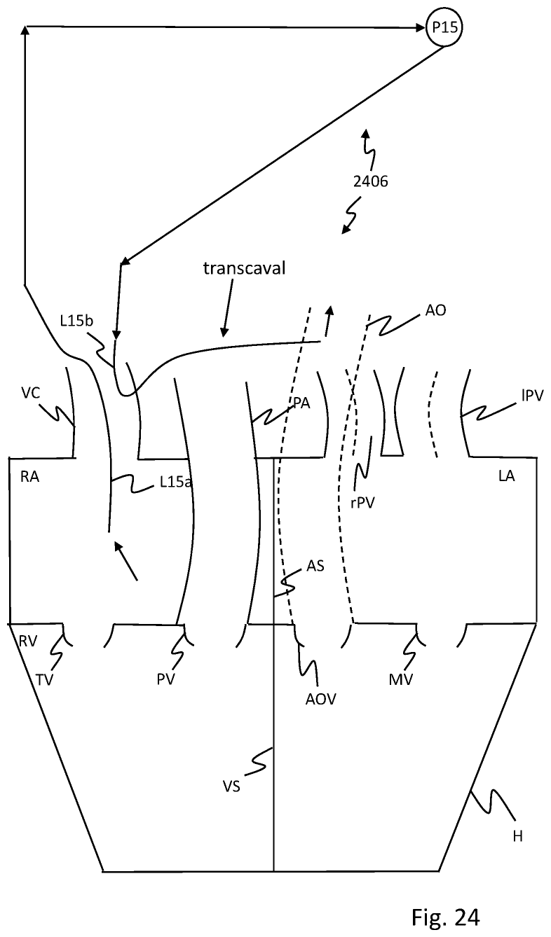

FIG. 24 an alternative embodiment wherein a cannula is punctured transcaval from vena cava to the aorta,

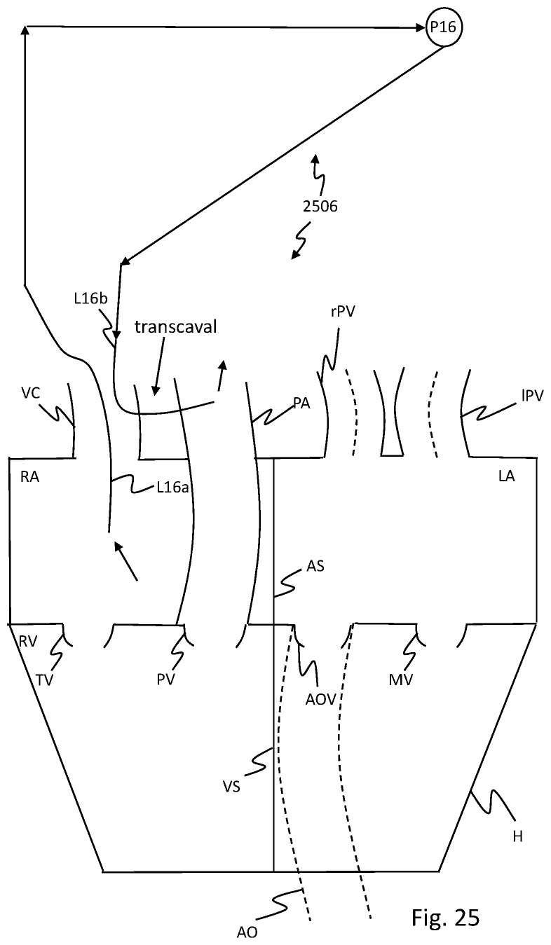

FIG. 25 a further alternative embodiment wherein a cannula is punctured transcaval from vena cava to the pulmonary artery,

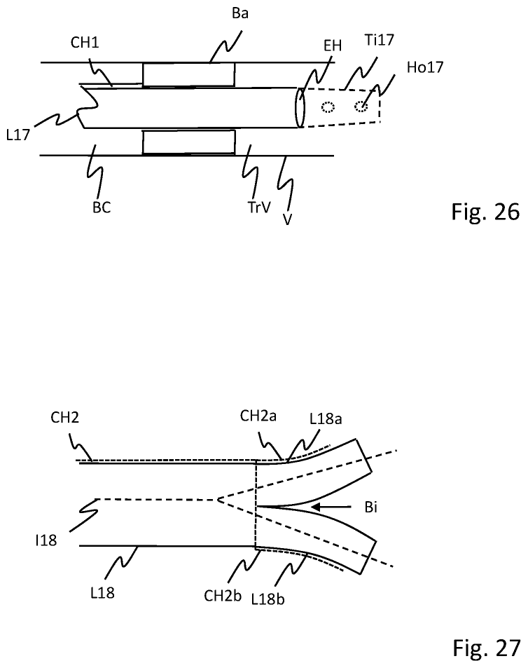

FIG. 26 a cannula that carries an inflatable expandable arrangement,

FIG. 27 a split tip cannula that carries two expandable arrangements,

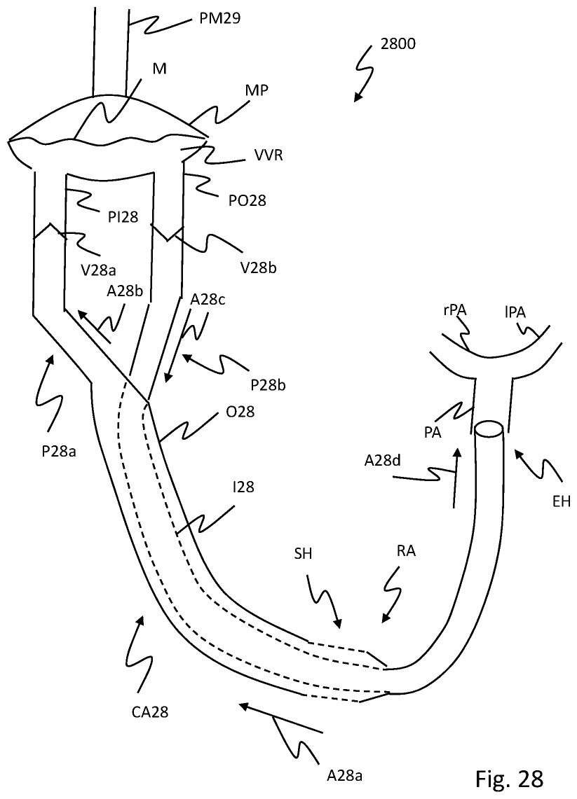

FIG. 28 a blood circulating system comprising a dual lumen insertable cannula and a membrane pump,

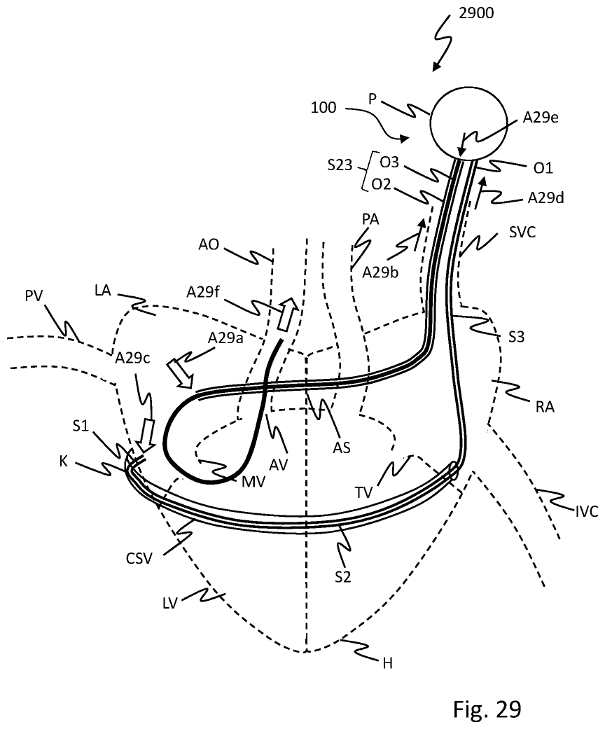

FIG. 29 a heart assist system comprising a dual cannula and a cannula within the coronary sinus,

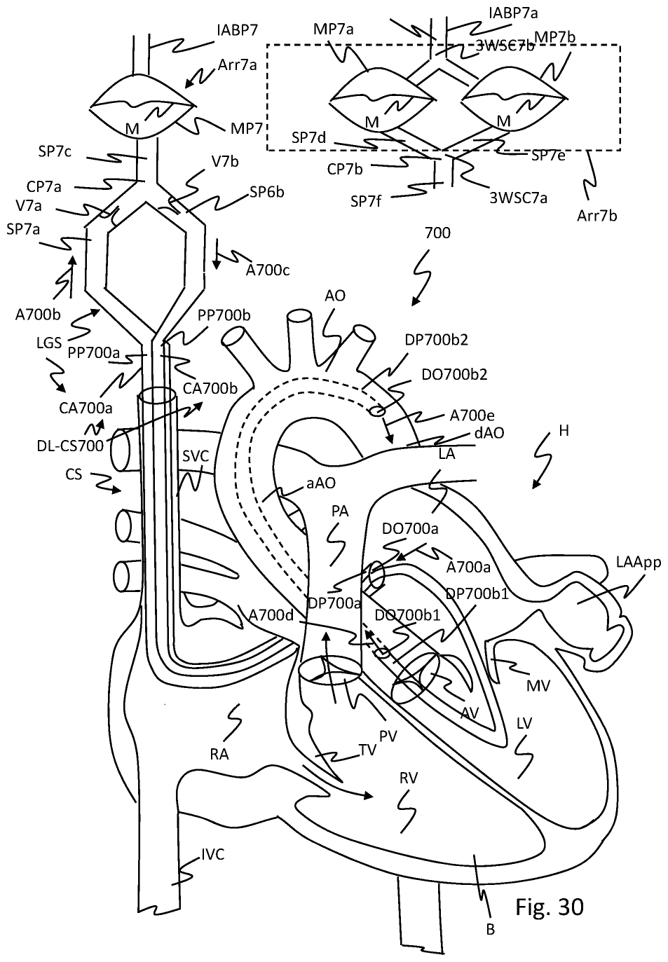

FIG. 30 a pLVAD® assist system with blood transport from left atrium to aorta, e.g. to ascending aorta or to descending aorta, with a dual lumen cannula system,

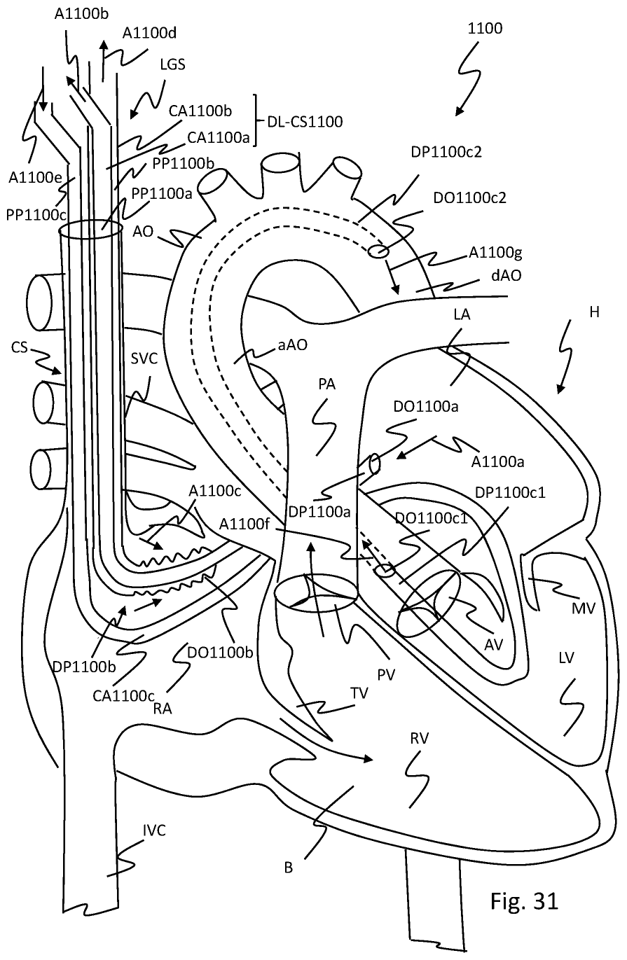

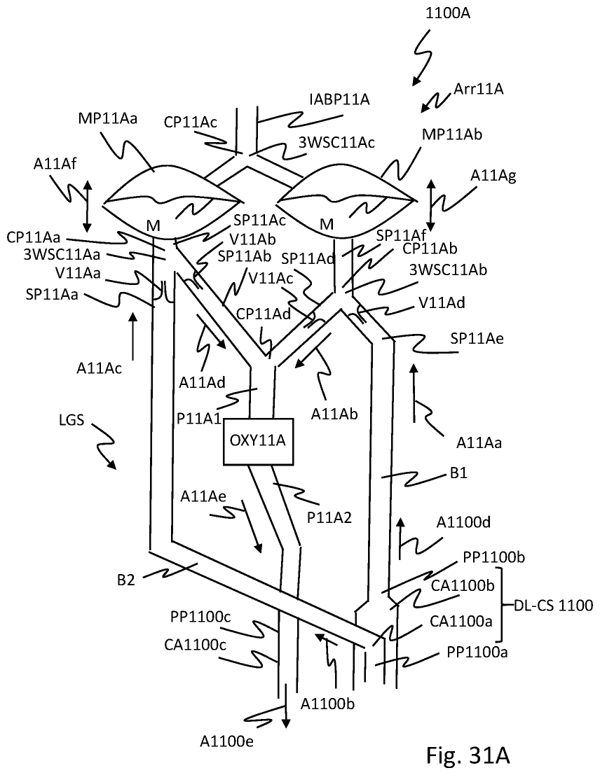

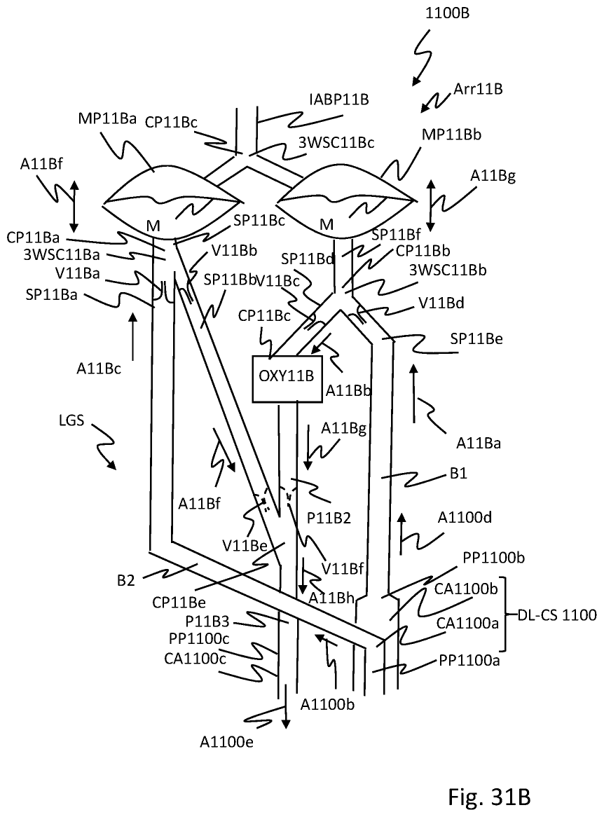

FIG. 31 a pBiVAD® assist system with blood transport from left atrium and right atrium to aorta, e.g. to ascending aorta or to descending aorta, using a dual lumen cannula system and one single lumen cannula (separate),

FIG. 31A a first embodiment of a pump arrangement for the pBiVAD® assist system of FIG. 11 including an oxygenator device, and

FIG. 31B a second embodiment of a pump arrangement for the pBiVAD® assist system of FIG. 11 including an oxygenator device and allowing pulsatile outflow.

### A) LEFT AND BI VENTRICLE ASSIST

FIG. 1 illustrates an extra corporeal circular blood flow circuitry 106 comprising a single lumen cannula 110 carrying a cage arrangement 116 near at least one inlet port, a blood pump P1 and a second single lumen cannula 140 that has at least one outlet port in the artery of the lower trunk. First single lumen cannula 110 is inserted through right internal jugular vein IJV, superior vena cava SVC, right atrium RA, transseptal, i.e. through the septum between right atrium RA and left atrium LA, into left atrium LA. A guide wire (not shown) may be used to guide cannula 110 to its final position. Alternatively, cannula 110 may be inserted through right subclavian vein. Blood is withdrawn by suction from left atrium LA through cannula 110, see arrows 160, 162.

Cannula 140 is inserted through the right femoral artery into common femoral artery CFA where blood is injected in a retrograde fashion into common femoral artery CFA, see arrows 170 and 172.

A body 100 comprises a head 102 and a trunk 104, see FIG. 4. The heart H of a patient is located within trunk 104. The patient may be a male or female adult or a child. The heart H comprises the following chambers:
right atrium RA,
right ventricle RV,
left atrium LA, and
left ventricle LV.

The atrial septum is between right atrium RA and left atrium LA. The ventricle septum is between right ventricle RV and left ventricle LV.

The following valves of heart H are shown:
tricuspid valve TVa between right atrium RA and right ventricle RV, and
mitral valve MVa.

The aortic valve AVa between aorta AO and left ventricle LV is not shown. The same applies for pulmonary valve PVa between right ventricle RV and pulmonary artery PA that is omitted in order to not to obscure the view to the parts of heart H that are relevant in the shown embodiment. Left pulmonary vein PV is shown in FIG. 1. Blood that is enriched with oxygen comes from lung L into left atrium LA through pulmonary vein PV. This is an exception in that a vein transports blood that comprises more oxygen than blood in a comparable artery. The description of heart H will not be repeated below. However, it is clear that this description is valid for all FIGS. 1 to 11.

An optional inlet tip 114 may be mounted on distal end 112 of cannula 110. Inlet tip 114 may comprise a plurality of inlet holes 115 in its side wall. Additionally, there may be a hole within distal end 112 of inlet tip 114. The sum of the cross section areas of the holes of tip 114 may be greater than the inner cross section area of cannula 110 at its distal end 112, for instance greater than twice the area or the triple of the area. This means that blood can be removed even if one or more of the inlet holes 115 in inlet tip 114 is or are clogged.

However, in other embodiments no separate inlet tip 114 is used. Thus, there is only one inlet hole at distal end 112 of cannula 110. This single inlet hole would be surrounded by cage arrangement 116.

Cage arrangement 116 is one possible example. Other possible examples are described below with reference to FIGS. 16 to 20. Cage arrangement 116 may comprise for instance between 6 to 12 flexible wires, beams or bars. There may be for instance 8 cage wires 118 that span a sphere. The sphere prevents that the side wall of left atrium LA covers one of inlet holes 115 of tip 114. Furthermore, cage arrangement 116 fixes distal end 112 of cannula 110 to the septum. Thus it is not possible that cannula 110 slides back into right atrium RA.

With reference again to FIG. 1, a tube 120 is connected to a proximal end of cannula 110 and to an inlet of pump P1. Pump P1 may be a peristaltic pump or centrifugal pump or another kind of pump, for instance a membrane pump or an axial pump. A tube 130 is connected to an outlet of pump P1 and to the proximal end of cannula 140. Pump P1 may be an electrically driven pump. There may be a control unit that controls the pumping performance, for instance depending on an ECG (electrocardiography) signal. Pump P1 may be operated in pulsed or continuous mode.

Tubes 120, 130 may be made of a flexible material or of a more rigid material. Circuitry 306 may further include one or more blood filter units or units for dialysis of blood.

Cannula 140 may comprise an optional outlet tip 150 that has the same structure as inlet tip 114 of cannula 110. This means that outlet tip 150 may comprise a plurality of outlet holes 152 in its side wall and/or on its distal end.

Extra care has to be taken because cannula 140 is inserted into an artery. Blood pressure is much higher in an artery compared to blood pressure in vein. Furthermore, blood flow from a vein is continuously but blood flow in artery is pulsed. Pulsed mode of the pump is not necessary because of the retrograde infusion.

However, pump P1 may be operated in a pulsed mode. Control may be performed depending on the rhythm of the heartbeat. A sensor may be used to detect the heartbeat, especially an electronic sensor. If heart H is in a diastolic state the counter pressure against infusion of blood into artery CFA may be weak.

Retrograde infusion of blood may not be as advantageous as antegrade infusion because water divides of the lymphatic system are formed and because of the forming of turbulences. The formation of thrombus may be facilitated by retrograde infusion. The arrangement shown in FIG. 1 may be used for patients without lung problems. Furthermore, circuitry 106 supports the left part of heart H of the patient. The arrangement shown in FIG. 1 may be named pLVAD (percutaneous left ventricle assisted device).

FIG. 2 illustrates an extra corporeal circular blood flow circuitry 206 comprising a dual lumen cannula 210 carrying a cage arrangement 216 near at least one outlet port and a blood pump P2. Cannula 210 is inserted through right internal jugular vein IJV, superior vena cava SVC, right atrium RA, transseptal, i.e. through the septum between right atrium RA and left atrium LA, into left atrium LA. Then, from left atrium LA through mitral valve MVa, left ventricle LV, through aortic valve AVa into ascending aorta aAO. A guide wire (not shown) may be used to guide cannula 210 to its final position. Alternatively, cannula 210 may be inserted through the right subclavian vein.

Blood is withdrawn by suction from left atrium LA through an outer lumen of cannula 210, see arrows 260, 262.

Blood is pumped into ascending aorta aAO through an inner lumen of cannula 210, see arrows 270, 272. Blood may be pumped in in a pulsed mode, preferably every time aortic valve AVa is closed. During the diastole, i.e. the heart refills with blood, there may be a first ejection of blood and during systole, i.e. contraction, there may be a normal or second ejection of blood out of the outlet holes of inner lumen of cannula 210. Alternatively, blood is only ejected during the systole.

Further to FIG. 2, a tube 220 is connected to a proximal end of the outer lumen of cannula 210 and to an inlet of pump P2. Pump P2 may be a peristaltic pump or centrifugal pump or another kind of pump, for instance a membrane pump. A tube 230 is connected to an outlet of pump P2 and to the proximal end of the inner lumen of cannula 210.

Pump P2 may be an electrically driven pump. There may be a control unit that controls the pumping performance, for instance depending on an ECG (electrocardiography) signal. Tubes 220, 230 may be made of a flexible material or of a more rigid material. Alternatively, pump P2 may be operated in continuous mode. Circuitry 206 may further include one or more blood filter units or units for dialysis of blood.

There may be a group of inlet holes 252 within the sidewall of the outer lumen within an intermediate portion of cannula 210, especially within an inlet portion 250 of cannula 210. Inlet holes 252 may be arranged circumferentially on all sides of cannula 210. Inlet holes 252 may be arranged at a location of cannula 210 that is within left atrium LA if cannula 210 is arranged in place as shown in FIG. 2. The outer lumen of cannula 210 may or may not extend distally beyond inlet holes 252. Furthermore, it is possible to reduce the outer diameter of cannula 210 distally beyond inlet portion 250.

Cage arrangement 216 is one possible example. Other possible examples are described below with reference to FIGS. 16 to 20. Cage arrangement 216 may comprise for instance between 6 to 12 flexible wires, beams or bars. There may be for instance 8 cage wires 218 that span a sphere. The sphere prevents that the side wall of the ascending aorta aAO covers one of the holes of tip 214. However, this is also prevented by the blood that is pumped out of distal end 212. Furthermore, cage arrangement 216 fixes distal end 212 of cannula 210 within ascending aorta aAO. Thus it is not possible that cannula 210 slides back through aortic valve AVa into left ventricle LV.

The patient is able to walk because there are no cannulas in his legs or his groin. Furthermore, circuitry 206 supports the left part of heart H. The arrangement shown in FIG. 2 may be named pLVAD DL (percutaneous left ventricle assisted device dual lumen). The dual lumen cannula may be formed as shown. Alternatively, a dual lumen cannula may be used that is described in more detail below with regard to FIGS. 12 to 15.

FIG. 3 illustrates an extra corporeal circular blood flow circuitry 306 comprising a dual lumen cannula 310 carrying a cage arrangement 316 near at least one outlet port, a blood pump P3 and an oxygenator OXY3. Cannula 310 is inserted endovascularly through the right internal jugular vein IJV, superior vena cava SVC, right atrium RA, transseptal, i.e. through the septum between right atrium RA and left atrium LA, into left atrium LA. Then, from left atrium LA through mitral valve MVa, left ventricle LV, through aortic valve AVa into ascending aorta aAO. A guide wire (not shown) may be used to guide cannula 310 to its final position. Alternatively, cannula 310 may be inserted through the right subclavian vein.

Blood is withdrawn by suction from right atrium RA through an outer lumen of cannula 310, see arrows 360, 362.

Blood is pumped into the ascending aorta aAO through an inner lumen of cannula 310, see arrows 370, 372. Blood may be pumped in in a pulsed mode, preferably every time aortic valve AVa is closed. During the diastole, i.e. the heart refills with blood, there may be a first ejection of blood and during systole, i.e. contraction, there may be a normal or second ejection of blood out of the outlet holes of inner lumen of cannula 310. Alternatively, blood is only ejected during the systole. Alternatively, a continuous blood flow may be generated.

With reference further to FIG. 3, a tube 320 is connected to a proximal end of the outer lumen of cannula 210 and to an inlet of pump P3. Pump P3 may be a peristaltic pump or centrifugal pump or another kind of pump, for instance a membrane pump. A tube 340 is connected to an outlet of pump P2 and to the inlet of an oxygenator device OXY3. Oxygenator device OXY3 is used to enrich the oxygen content of the blood. Alternatively or additionally, oxygenator device OXY3 may also reduce carbon dioxide within blood. An outlet of Oxygenator OXY3 is connected to the proximal end of inner lumen of cannula 310 by a tube 330.

Pump P3 may be an electrically driven pump. There may be a control unit that controls the pumping performance, for instance depending on an ECG (electrocardiography) signal. Alternatively, pump P3 may be operated in continuous mode. Tubes 220, 230 may be made of a flexible material or of a more rigid material. Circuitry 306 may further include one or more blood filter units or units for dialysis of blood.

There may be a group of inlet holes 352 within the sidewall of the outer lumen of cannula 310 within an intermediate portion of cannula 310, especially within an inlet portion 350 of cannula 310. Inlet holes 352 may be arranged circumferentially on all sides of cannula 310. Inlet holes 352 may be arranged at a location of cannula 310 that is within right atrium RA if cannula 310 is arranged in place as shown in FIG. 3. An optional second inlet portion 354 may be arranged more distally than inlet portion 350. However, the inlet holes of second inlet portion 354 may also be arranged at a location of cannula 310 that is within right atrium RA if cannula 310 is arranged in place as shown in FIG. 3. The outer lumen of cannula 310 may or may not extend distally beyond the inlet holes 252. Furthermore, it is possible to reduce the outer diameter of cannula 310 distally beyond inlet portion 350 or beyond inlet portion 354.

Cage arrangement 316 is one possible example. Other possible example are described below with reference to FIGS. 16 to 20. Cage arrangement 316 may comprise for instance between 6 to 12 flexible wires, beams or bars. There may be for instance 8 cage wires 318 that span a sphere. The sphere prevents that the side wall of the ascending aorta aAO covers one of the holes of tip 314. However, this is also prevented by the blood that is pumped out of distal end 312. Furthermore, cage arrangement 316 fixes distal end 312 of cannula 310 within ascending aorta aAO. Thus it is not possible that cannula 310 slides back through aortic valve AVa into left ventricle LV.

The patient is able to walk because there are no cannulas in his legs or his groin. Furthermore, circuitry 306 supports both the left part and the right part of heart H. The arrangement shown in FIG. 3 may be named as pBiVAD DL (percutaneous bi ventricle assist device dual lumen). Dual lumen cannula 310 may be formed as shown. Alternatively, a dual lumen cannula may be used that is described in more detail below with regard to FIGS. 12 to 15.

FIG. 4 illustrates a transcaval extra corporeal circular blood flow circuitry 406 comprising a single lumen cannula 410 carrying a cage arrangement 416 near at least one inlet port, a blood pump P4 and a second single lumen cannula 440 that has at least one outlet port in the artery of the lower trunk. First single lumen cannula 410 is inserted through the right internal jugular vein IJV, superior vena cava SVC, right atrium RA, trans-septal, i.e. through the septum between right atrium RA and left atrium LA, into left atrium LA. A guide wire (not shown) may be used to guide cannula 410 to its final position. Alternatively, cannula 410 may be inserted through the right subclavian vein. Blood is withdrawn by suction from left atrium LA through cannula 410, see arrows 460, 462.

Cannula 440 is inserted through the right femoral vein into the common femoral vein CFV and then transcaval via a transcaval passage 480 into common femoral artery CFA where blood is injected in a retrograde fashion into common femoral artery CFA, see arrows 470 and 472. Means may be used in order to support the vein and/or the artery openings that are part of transcaval passage 480. These means may be left within body 100 after removing cannula 440 for further uses. An example for such means is a fixation set that is available within the market.

Body 100 comprises a head 102 and a trunk 104. Heart H of a patient is located within trunk 104. The patient may be a male or female adult or a child. The description of the heart is given above with regard to FIG. 1. This description is valid for all FIGS. 1 to 15.

An optional inlet tip 414 may be mounted on distal end 412 of cannula 410. Inlet tip 414 may comprise a plurality of inlet holes 415 in its side wall. Additionally, there may be a hole within the distal end of inlet tip 414. The sum of the cross section areas of the holes of tip 414 may be greater than the inner cross section area of cannula 410 at its distal end 112, for instance greater than twice the area or the triple of the area. This means that blood can be removed even if one or more of the inlet holes 415 in inlet tip 414 is or are clogged.

However, in other embodiments no separate inlet tip 414 is used. Thus, there is only one inlet hole at distal end 412 of cannula 410. This single inlet hole would be surrounded by cage arrangement 416.

Cage arrangement 416 is one possible example. Other possible example are described below with reference to FIGS. 16 to 20. Cage arrangement 416 may comprise for instance between 6 to 12 flexible wires, beams or bars. There may be for instance 8 cage wires 118 that span a sphere. The sphere prevents that the side wall of left atrium LA covers one of inlet holes 415 of tip 414.

Furthermore, cage arrangement 416 fixes distal end 412 of cannula 410 to the septum. Thus it is not possible that cannula 410 slides back into right atrium RA.

With reference further to FIG. 4, a tube 420 is connected to a proximal end of cannula 410 and to an inlet of pump P4. Pump P4 may be a peristaltic pump or centrifugal pump or another kind of pump. A tube 430 is connected to an outlet of pump P4 and to the proximal end of cannula 440. Pump P4 may be an electrically driven pump. There may be a control unit that controls the pumping performance, for instance depending on an ECG (electrocardiography) signal. Pump P4 may be operated in pulsed or continuous mode.

Tubes 420, 430 may be made of a flexible material or of a more rigid material. The circuitry 406 may further include one or more blood filter units or units for dialysis of blood.

Cannula 440 may comprise an optional outlet tip 450 that has the same structure as inlet tip 414 of cannula 410. This means that outlet tip 450 may comprise a plurality of outlet holes 452 in its side wall and/or on its distal end. Additionally, cannula 440 may comprise a cage arrangement on its distal end 442. The cage arrangement may be formed as described above for instance for cage arrangement 116 or cage arrangement 546, see FIG. 5 and corresponding description.

No extra care has to be taken because cannula 440 is inserted first into a vein in which there is comparably low blood pressure. However, the transcaval passage 480 has to be handled with care because blood pressure is much higher in an artery compared to blood pressure in a vein. Furthermore, blood flow in a vein is continuously but blood flow in an artery is pulsed. Pulsed mode of the pump is not necessary because of the retrograde infusion.

However, pump P4 may be operated in a pulsed mode. Control may be performed depending on the rhythm of the heartbeat. A sensor may be used to detect the heartbeat, especially an electronic sensor. If the heart is in a diastolic state the counter pressure against infusion of blood into artery CFA may be weak.

Retrograde infusion of blood may not be as advantageous as antegrade infusion because of water divides of the lymphatic system that may be formed and because of the forming of turbulences. The formation of thrombus may be facilitated by retrograde infusion. The arrangement shown in FIG. 4 may be used for patients without lung problems. Furthermore, circuitry 406 supports the left part of heart H of the patient. The arrangement shown in FIG. 1 may be named pLVAD transcaval (percutaneous left ventricle assisted device).

FIG. 5 illustrates a transcaval extra corporeal circular blood flow circuitry 506 comprising a single lumen cannula 510 carrying a cage arrangement 516 near at least one inlet port, a blood pump P5 and a second single lumen cannula 540 that has at least one outlet port in the artery of the lower trunk 104. First single lumen cannula 510 is inserted through right internal jugular vein IJV, superior vena cava SVC, right atrium RA, transseptal, i.e. through the septum between right atrium RA and left atrium LA, into left atrium LA. A guide wire (not shown) may be used to guide cannula 510 to its final position. Alternatively, cannula 510 may be inserted through the right subclavian vein. Blood is withdrawn by suction from left atrium LA through cannula 510, see arrows 560, 562.

Cannula 540 is inserted through the left internal jugular vein IN, superior vena cava SVC, right atrium RA, inferior vena cava IVC and then transcaval via a transcaval passage 580 into common femoral artery CFA where blood is injected in a retrograde fashion into common femoral artery CFA, see arrow 570. Means may be used in order to support the vein and/or the artery openings that are part of transcaval passage 580. These means may be left within body 100 after removing cannula 540 for further uses. An example for such means is a fixation set that is available within the market.

An optional inlet tip 514 may be mounted on distal end 512 of cannula 510. Inlet tip 514 may comprise a plurality of inlet holes 515 in its side wall. Additionally, there may be a hole within the distal end of inlet tip 514. The sum of the cross section areas of the holes of tip 514 may be greater than the inner cross section area of cannula 510 at its distal end 512, for instance greater than twice the area or the triple of the area. This means that blood can be removed even if one or more of the inlet holes 515 in inlet tip 514 is or are clogged.

However, in other embodiments no separate inlet tip 514 is used. Thus, there is only one inlet hole at the distal end 512 of cannula 510. This single inlet hole would be surrounded by cage arrangement 516.

Cage arrangement 516 is one possible example. Other possible examples are described below with reference to FIGS. 16 to 20. Cage arrangement 516 may comprise for instance between 6 to 12 flexible wires, beams or bars. There may be for instance 8 cage wires 518 that span a sphere. The sphere prevents that the side wall of left atrium LA covers one of inlet holes 515 of tip 514. Furthermore, cage arrangement 516 fixes distal end 512 of cannula 510 to the septum. Thus it is not possible that cannula 510 slides back into right atrium RA.

Further to FIG. 5, a tube 520 is connected to a proximal end of cannula 510 and to an inlet of pump P5. Pump P5 may be a peristaltic pump or centrifugal pump or another kind of pump, for instance a membrane pump. A tube 530 is connected to an outlet of pump P5 and to the proximal end of cannula 540. Pump P5 may be an electrically driven pump. There may be a control unit that controls the pumping performance, for instance depending on an ECG (electrocardiography) signal. Pump P5 may be operated in pulsed or continuous mode.

Tubes 520, 530 may be made of a flexible material or of a more rigid material. Circuitry 506 may further include one or more blood filter units or units for dialysis of blood.

Cannula 540 may comprise an optional outlet tip 550 that may have the same structure as inlet tip 514 of cannula 510. This means that outlet tip 550 may comprise a plurality of outlet holes 452 in its side wall and/or on its distal end. Additionally, cannula 540 may have an optional cage arrangement 546 on its distal end 542.

Cage arrangement 546 is one possible example. Other possible examples are described below with reference to FIGS. 16 to 20. Cage arrangement 546 may comprise for instance between 6 to 12 flexible wires, beams or bars. There may be for instance 8 cage wires 548 that span a sphere. The sphere prevents that the side wall of common femoral artery CFA covers one of outlet holes 552 of optional outlet tip 550. Furthermore, cage arrangement 546 fixes distal end 542 of cannula 540 within common femoral artery CFA and allows antegrade blood flow of blood coming from heart H and/or from outlet holes 552.

No extra care has to be taken because cannula 540 is inserted first into a vein in which there is comparably low blood pressure. However, transcaval passage 580 has to be handled with care because blood pressure is much higher in an artery compared to blood pressure in a vein. Furthermore, blood flow from a vein is continuously but blood flow in an artery is pulsed. Pulsed mode of the pump is not necessary because of the retrograde infusion.

However, pump P5 may be operated in a pulsed mode. Control may be performed depending on the rhythm of the heartbeat of heart H. A sensor may be used to detect the heartbeat, especially an electronic sensor. If heart H is in a diastolic state the counter pressure against infusion of blood into common femoral artery CFA may be weak.

Retrograde infusion of blood may not be as advantageous as antegrade infusion because of water divides of the lymphatic system and of the forming of turbulences. The formation of thrombus may be facilitated by retrograde infusion.

The arrangement shown in FIG. 5 may be used for patients without lung problems. Furthermore, circuitry 506 supports the left part of the heart H of the patient. Mobility of the patient is possible because no cannulas in femoral veins or arteries are used. Compared with FIG. 4, the arrangement of FIG. 5 allows blood injection more central within body 100. The arrangement shown in FIG. 5 may be named pLVAD transcaval (percutaneous left ventricle assisted device).

In other embodiments it is possible to insert cannula 510 through the left internal jugular vein IJV to the left atrium LA as described above and cannula 540 through the right internal jugular vein UV into the common femoral artery CFA.

### B) LUNG ASSIST

FIG. 6 illustrates an extra corporeal circular blood flow circuitry 606 comprising two single lumen cannulas 610 and 620 and a carbon dioxide removal device CO2R6 but no pump. Single lumen cannula 610 carries a cage arrangement 616 near at least one inlet port that is arranged in pulmonary artery PA. Second single lumen cannula 640 has at least one outlet port within left atrium LA.

First single lumen cannula 610 is inserted through right internal jugular vein UV, superior vena cava SVC, right atrium RA, right ventricle RV, through pulmonary valve PVa into pulmonary artery PA. A guide wire (not shown) may be used to guide cannula 610 to its final position. Alternatively, cannula 610 may be inserted through the right subclavian vein and then along the same way as described above. Blood is withdrawn by suction from pulmonary artery PA through cannula 610, see arrow 660. A part of the blood that comes from right ventricle RV is pumped by heart H into pulmonary artery PA and is enriched in the lung with oxygen. Removal of carbon dioxide may be necessary for instance for patients that have chronic obstructive pulmonary disease (COPD) or cystic fibrosis.

Cannula 640 is inserted through left internal jugular vein IJV, superior vena cava SVC, right atrium RA, trans-septal, i.e. through the septum between right atrium RA and left atrium LA, into left atrium LA. A guide wire (not shown) may be used to guide cannula 640 to its final position. Alternatively, cannula 610 may be inserted through the right subclavian vein.

An optional inlet tip 614 may be mounted on distal end 612 of cannula 610. Inlet tip 614 may comprise a plurality of inlet holes 615 in its side wall. Additionally, there may be a hole within the distal end of inlet tip 614. The sum of the cross section areas of the holes of tip 614 may be greater than the inner cross section area of cannula 610 at its distal end 612, for instance greater than twice the area or the triple of the area. This means that blood can be removed even if one or more of inlet holes 615 in inlet tip 614 is or are clogged.

However, in other embodiments no inlet tip 614 is used. Thus, there is only one inlet hole at the distal end 612 of cannula 610. This single inlet hole would be surrounded by cage arrangement 616.

Cage arrangement 616 is one possible example. Other possible example are described below with reference to FIGS. 16 to 20. Cage arrangement 616 may comprise for instance between 6 to 12 flexible wires, beams or bars. There may be for instance 8 cage wires 618 that span a sphere. The sphere prevents that the side wall of left atrium LA covers one of inlet holes 615 of tip 614. Furthermore, cage arrangement 616 fixes distal end 612 of cannula 510 to pulmonary artery PA. Thus, it is not possible that cannula 510 slides back through pulmonary valve PV into right ventricle RV.

Further to FIG. 6, a tube 620 is connected to a proximal end of cannula 610 and to an inlet of carbon dioxide removal device CO2R6 that removes carbon dioxide from the blood. A pump may not be necessary but may be used in another embodiment. A tube 630 is connected to an outlet of carbon dioxide removal device CO2R6 and to the proximal end of cannula 640. Carbon dioxide removal device CO2R6 may contain a semipermeable membrane.

Tubes 620, 630 may be made of a flexible material or of a more rigid material. Circuitry 606 may further include one or more blood filter units or units for dialysis of blood. However, the natural blood pressure may not be sufficient to press the blood also through a filter device without using a pump.

Cannula 640 may comprise an optional outlet tip 650 that may have the same structure as the inlet tip 614 of cannula 610. This means that outlet tip 650 may comprise a plurality of outlet holes 652 in its side wall and/or on its distal end. Additionally, cannula 640 may have an optional cage arrangement 646 on its distal end 642. Blood with less carbon dioxide is injected into the left atrium LA through cannula 640 and mixes with oxygen rich blood that comes through the pulmonary veins from the lung.

Cage arrangement 646 is one possible example. Other possible examples are described below with reference to FIGS. 16 to 20. Cage arrangement 646 may comprise for instance between 6 to 12 flexible wires, beams or bars. There may be for instance 8 cage wires 648 that span a sphere. The sphere and also the expelled blood prevent that the side wall of left atrium LA covers one of the outlet holes 652 of optional outlet tip 650. Furthermore, cage arrangement 646 fixes distal end 642 of cannula 640 within left atrium LA.

No extra care has to be taken because both cannulas 510 and 540 are inserted into veins in which there is comparably low blood pressure.

Antegrade infusion is performed that has many advantages, i.e. no forming of water divides of the lymphatic system and less forming of turbulences. The formation of thrombus may be prevented by antegrade infusion.

The arrangement shown in FIG. 6 may be used for patients with lung problems. Mobility of the patient is possible because no cannulas in femoral veins or arteries are used. The arrangement shown in FIG. 6 may be named pECLA (percutaneous left extra corporeal lung assist).

In other embodiments it is possible to insert cannula 610 through left internal jugular vein IJV/left subclavian vein to pulmonary artery PA as described above and cannula 640 through right internal jugular vein IJV/right subclavian vein to left atrium LA.

In another embodiment a pump is connected in series with carbon dioxide removal device CO2R6. This allows to remove more carbon dioxide from the blood, for instance more than 30 percent compared to the content on the inlet of the carbon dioxide removal device CO2R6. This embodiment may be named ECCO₂R (extracorporeal CO₂ removal).

In a further embodiment an oxygenator device is used instead of carbon dioxide removal device CO2R6 and preferably a pump is connected in series with the oxygenator. The oxygenator device enriches the oxygen content in the blood and decreases the carbon dioxide content at the same time. This further embodiment may be named ECMO (extracorporeal membrane oxygenation).

FIG. 7 illustrates a transcaval extra corporeal lung assist circular blood flow circuitry 706 comprising a single lumen cannula 710 carrying a cage arrangement 716 near at least one inlet port, a carbon dioxide removal device CO2R7 and a single lumen cannula 740 that has at least one outlet port in the right atrium RA.

Cannula 710 is inserted through right internal jugular vein IN, superior vena cava SVC, right atrium RA, inferior vena cava IVC and then transcaval via a transcaval passage 780 into common femoral artery CFA where blood with comparably high oxygen content is withdrawn from, see arrows 760, 762. A guide wire (not shown) may be used to guide cannula 710 to its final position. Alternatively, cannula 710 may be inserted through the right subclavian vein. Means may be used in order to support the vein and/or the artery openings that are part of transcaval passage 780. These means may be left within body 100 after removing cannula 710 for further uses. An example for such means is a fixation set that is available within the market.

Single lumen cannula 740 is inserted through left internal jugular vein IJV, superior vena cava SVC into right atrium RA. A guide wire (not shown) may be used to guide cannula 710 to its final position. Alternatively, cannula 710 may be inserted through right subclavian vein. Blood with reduced carbon dioxide content is ejected into the right atrium RA through cannula 740, see arrows 770, 772. This blood is then pumped by heart H through right ventricle RV and pulmonary artery PA, see FIG. 6, to lung L of the patient having body 100.

An optional inlet tip 714 may be mounted on distal end 712 of cannula 710. Inlet tip 714 may comprise a plurality of inlet holes 715 in its side wall. Additionally, there may be a hole within the distal end of inlet tip 714. The sum of the cross-section areas of the holes of tip 714 may be greater than the inner cross section area of cannula 710 at its distal end 712, for instance greater than twice the area or the triple of the area. This means that blood can be removed even if one or more of the inlet holes 715 in inlet tip 714 is or are clogged.

However, in other embodiments no inlet tip 714 is used. Thus, there is only one inlet hole at distal end 712 of cannula 710. This single inlet hole would be surrounded by cage arrangement 716.

Cage arrangement 716 is one possible example. Other possible examples are described below with reference to FIGS. 16 to 20. Cage arrangement 716 may comprise for instance between 6 to 12 flexible wires, beams or bars. There may be for instance 8 cage wires 718 that span a sphere. The sphere prevents that the side wall of common femoral artery CFA covers one of inlet holes 715 of tip 714. Furthermore, cage arrangement 716 fixes distal end 712 of cannula 710 to common femoral artery CFA. Thus it is not possible that cannula 710 slides back into transcaval passage 780.

With reference further to FIG. 7, a tube 720 is connected to a proximal end of cannula 710 and to an inlet of carbon dioxide removal device CO2R7. Carbon dioxide removal device CO2R7 may comprise a semipermeable membrane. A tube 730 is connected to an outlet of carbon dioxide removal device CO2R7 and to the proximal end of cannula 840.

Tubes 720, 730 may be made of a flexible material or of a more rigid material. Circuitry 706 may further include one or more blood filter units or units for dialysis of blood. However, an additional pump may be necessary if a filter unit/dialysis unit is used.

Cannula 740 may comprise an optional outlet tip 750 that may have the same structure as inlet tip 714 of cannula 710. This means that outlet tip 750 may comprise a plurality of outlet holes 752 in its side wall and/or on its distal end. Additionally, cannula 740 may have an optional cage arrangement 746 on its distal end 742.

Cage arrangement 746 is one possible example. Other possible examples are described below with reference to FIGS. 16 to 20. Cage arrangement 746 may comprise for instance between 6 to 12 flexible wires, beams or bars. There may be for instance 8 cage wires 548 that span a sphere. The sphere prevents that the side wall of common femoral artery CFA covers one of outlet holes 752 of optional outlet tip 750. Furthermore, cage arrangement 746 fixes distal end 742 of cannula 740 within right atrium RA.

No extra care has to be taken because both cannulas 710 and 740 are inserted first into a vein in which there is comparably low blood pressure. However, transcaval passage 780 has to be handled with care because blood pressure is much higher in an artery compared to blood pressure in a vein. Furthermore, blood flow from a vein is continuously but blood flow in an artery is pulsed.

Antegrade infusion is performed that has many advantages, i.e. no forming of water divides of the lymphatic system may occur and less forming of turbulences may be present. The formation of thrombus may be prevented by antegrade infusion.

The arrangement shown in FIG. 7 may be used for patients with lung problems. Mobility of the patient is possible because no cannulas in femoral veins or arteries are used. The arrangement shown in FIG. 7 may be named pECLA (percutaneous left extra corporeal lung assist) transcaval.

In other embodiments it is possible to insert cannula 710 through left internal jugular vein IJV/left subclavian vein to common femoral artery CFA as described above and cannula 740 through right internal jugular vein IN into right atrium RA.

In another embodiment a pump is connected in series with carbon dioxide removal device CO2R7. This allows to remove more carbon dioxide from the blood, for instance more than 30 percent compared to the content on the inlet of the carbon dioxide removal device CO2R7.

In a further embodiment an oxygenator device is used instead of carbon dioxide removal device CO2R7 and preferably a pump is connected in series with the oxygenator. The oxygenator device enriches the oxygen content in the blood and decreases the carbon dioxide content at the same time.

FIG. 8 illustrates a transcaval transseptal extra corporeal lung assist circular blood flow circuitry 806 comprising a single lumen cannula 810 carrying a cage arrangement 816 near at least one inlet port, a carbon dioxide removal device CO2R8 and a single lumen cannula 840 that has at least one outlet port in the left atrium LA.

Cannula 810 is inserted through the right internal jugular vein IJV, superior vena cava SVC, right atrium RA, inferior vena cava IVC and then transcaval via a transcaval passage 880 into common femoral artery CFA where blood with comparably high oxygen content is withdrawn from, see arrows 860, 862. A guide wire (not shown) and/or snares may be used to guide cannula 810 to its final position. Alternatively, cannula 810 may be inserted through the right subclavian vein. Means may be used in order to support the vein and/or the artery openings that are part of transcaval passage 880. These means may be left within body 100 after removing cannula 810 for further uses. An example for such means is a fixation set that is available within the market.

Single lumen cannula 840 is inserted through the left internal jugular vein IJV, superior vena cava SVC, right atrium RA, trans-septal, i.e. through the septum between right atrium RA and left atrium LA, into left atrium LA. A guide wire (not shown) may be used to guide cannula 810 to its final position. Alternatively, the cannula 810 may be inserted through the right subclavian vein. Blood with reduced carbon dioxide content is ejected into left atrium LA through cannula 840, see arrow 870. This blood is then pumped by heart H through right ventricle RV and pulmonary artery PA, see FIG. 6, to lung L of the patient having body 100.

An optional inlet tip 814 may be mounted on distal end 812 of cannula 810. Inlet tip 814 may comprise a plurality of inlet holes 815 in its side wall. Additionally, there may be a hole within the distal end of inlet tip 814. The sum of the cross section areas of the holes of tip 814 may be greater than the inner cross section area of cannula 810 at its distal end 812, for instance greater than twice the area or the triple of the area. This means that blood can be removed even if one or more of the inlet holes 815 in inlet tip 814 is or are clogged.

However, in other embodiments no inlet tip 814 is used. Thus, there is only one inlet hole at distal end 812 of cannula 810. This single inlet hole would be surrounded by cage arrangement 816.

Cage arrangement 816 is one possible example. Other possible examples are described below with reference to FIGS. 16 to 20. Cage arrangement 816 may comprise for instance between 6 to 12 flexible wires, beams or bars. There may be for instance 8 cage wires 818 that span a sphere. The sphere prevents that the side wall of common femoral artery CFA covers one of inlet holes 815 of tip 814. Furthermore, cage arrangement 816 fixes distal end 812 of cannula 710 within common femoral artery CFA. Thus it is not possible that cannula 810 slides unintentionally back into transcaval passage 880.

Further to FIG. 8, a tube 820 is connected to a proximal end of cannula 810 and to an inlet of carbon dioxide removal device CO2R8. Carbon dioxide removal device CO2R8 may comprise a semipermeable membrane. A tube 830 is connected to an outlet of carbon dioxide removal device CO2R8 and to the proximal end of cannula 840.

Tubes 820, 830 may be made of a flexible material or of a more rigid material. The circuitry 806 may further include one or more blood filter units or units for dialysis of blood. However, an additional pump may be necessary if a filter unit/dialysis unit is used.

Cannula 840 may comprise an optional outlet tip 850 that may have the same structure as the inlet tip 814 of cannula 810. This means that outlet tip 850 may comprise a plurality of outlet holes 852 in its side wall and/or on its distal end. Additionally, cannula 840 may have an optional cage arrangement 846 on its distal end 842.

Cage arrangement 846 is one possible example. Other possible examples are described below with reference to FIGS. 16 to 20. Cage arrangement 846 may comprise for instance between 6 to 12 flexible wires, beams or bars. There may be for instance 8 cage wires 848 that span a sphere. The sphere and the ejected blood prevent that the side wall of common femoral artery CFA covers one of outlet holes 852 of optional outlet tip 850. Furthermore, cage arrangement 846 fixes distal end 842 of cannula 840 within left atrium LA.

No extra care has to be taken because both cannulas 810 and 840 are inserted first into a vein in which there is comparably low blood pressure. However, transcaval passage 880 has to be handled with care because blood pressure is much higher in an artery compared to blood pressure in a vein. Furthermore, blood flow from a vein is continuously but blood flow in an artery is pulsed.

Antegrade infusion is performed that has many advantages, i.e. no forming of water divides of the lymphatic system and less forming of turbulences. The formation of thrombus may be prevented by antegrade infusion.

The arrangement shown in FIG. 8 may be used for patients with lung problems. Mobility of the patient is possible because no cannulas in femoral veins or arteries are used. The arrangement shown in FIG. 8 may be named pECLA (percutaneous left extra corporeal lung assist) transcaval transseptal.

In other embodiments it is possible to insert cannula 810 through left internal jugular vein IJV/left subclavian vein to common femoral artery CFA as described above and cannula 840 through right internal jugular vein IJV into right atrium RA.

In another embodiment a pump is connected in series with carbon dioxide removal device CO2R8. This allows to remove more carbon dioxide from the blood, for instance more than 30 percent compared to the content on the inlet of the carbon dioxide removal device CO2R8.

In a further embodiment an oxygenator is used instead of carbon dioxide removal device CO2R8 and preferably a pump is connected in series with the oxygenator device. The oxygenator device enriches the oxygen content in the blood and decreases the carbon dioxide content at the same time.

### C) LUNG PERFUSION

An isolation of the lung L is reached together with heart assist of heart H at the same moment for the circuitries that use percutaneous in-vivo lung perfusion (pIVLP). Thus, isolated perfusion and/or treatment of lung diseases is enabled, especially antegrade and/or retrograde, preferably also with switching between antegrade and retrograde or between retrograde and antegrade. However, if only a part of the lung is treated, the other part may function normal. There may be a lobe dedicated treatment or treatment of only a part of a lobe. This may allow to treat the lung L without heart H assist/support and or without lung support, e.g. without external blood oxygenation and/or without external carbon dioxide (CO₂) removal. Alternatively, partially or full heart H assist and/or lung L assist may be used even if only a part of the lung is treated, for instance.

FIG. 9 illustrates an extra corporeal lung perfusion circular blood flow circuitry 906 comprising two single lumen cannulas 910 and 940, a pump P9 and a further device D9. Single lumen cannula 910 carries a cage arrangement 916 near at least one inlet port that is arranged in left atrium LA. Second single lumen cannula 940 has at least one outlet port within pulmonary artery PA. Circuitry 906 is a more theoretical embodiment because in addition a heart assist would be necessary. However, there are many ways to realize such a heart assist. One possibility is described below with reference to FIG. 10.

Cannula 910 is inserted through the left internal jugular vein IJV, superior vena cava SVC, right atrium RA, trans-septal, i.e. through the atrial septum AS between right atrium RA and left atrium LA, into left atrium LA. A guide wire (not shown) may be used to guide cannula 910 to its final position. Alternatively, cannula 910 may be inserted through the right subclavian vein. Almost the whole blood that enters left atrium LA through the left and right pair of pulmonary veins PV may be taken in by cannula 910, see arrow 960, using a membrane 919 that is explained in more detail below.

The second single lumen cannula 940 is inserted through the right internal jugular vein IJV, superior vena cava SVC, right atrium RA, right ventricle RV, through pulmonary valve PVa into pulmonary artery PA. A guide wire (not shown) may be used to guide cannula 940 to its final position. Alternatively, cannula 940 may be inserted through the right subclavian vein and then along the same way as described above. Almost the whole blood that comes out of cannula 940 is injected into pulmonary artery PA, see arrow 970, using a membrane 949 that is explained in more detail below. Device D9 may be an injection device that injects a medicament or a treatment substance, for instance for treating lung cancer.

An optional inlet tip 914 may be mounted on distal end 912 of cannula 910. Inlet tip 914 may comprise a plurality of inlet holes 915 in its side wall. Additionally, there may be a hole within the distal end of inlet tip 914. The sum of the cross section areas of the holes of tip 914 may be greater than the inner cross section area of cannula 910 at its distal end 912, for instance greater than twice the area or the triple of the area. This means that blood can be removed even if one or more of inlet holes 915 in inlet tip 914 is or are clogged.

However, in other embodiments no inlet tip 914 is used. Thus, there is only one inlet hole at distal end 912 of cannula 910. This single inlet hole would be surrounded by cage arrangement 916. Using a cannula without a separate tip allows high flow rates of a fluid that is drained into the cannula 910. The cage arrangement 916 prevents that a wall of left atrium LA is sucked into the hole of cannula 910.

Cage arrangement 916 is one possible example. Other possible examples are described below with reference to FIGS. 16 to 20. Cage arrangement 916 may comprise for instance between 6 to 12 flexible wires, beams or bars. There may be for instance 8 cage wires 918 that span a sphere. The sphere prevents that the side wall of the left atrium LA covers one of inlet holes 915 of tip 914. Furthermore, cage arrangement 916 fixes distal end 912 of cannula 910 to the atrial septum AS. Thus it is not possible that cannula 910 slides back through the atrial septum into right atrium RA.

Membrane 919 may cover only one half of cage arrangement 916, e.g. a half that is defined by two cage wires 918 that are arranged opposite to each other or nearly opposite. Examples of membranes that may be used on cage arrangement 916 are described below with reference to FIGS. 16 to 20.

Further to FIG. 9, a tube 920 is connected to a proximal end of cannula 910 and to an inlet of pump P9. An outlet of pump P9 may be connected to an inlet of device D9. A tube 930 is connected to an outlet of device D9 and to the proximal end of cannula 940. Device D9 may be used for instance for injecting a drug or medicament or treatment substance into the lung of the patient.

Tubes 920, 930 may be made of a flexible material or of a more rigid material. The circuitry 906 may further include one or more blood filter units or units for dialysis of blood.

Cannula 940 may comprise an optional outlet tip 950 that may have the same structure as inlet tip 914 of cannula 910. This means that outlet tip 950 may comprise a plurality of outlet holes 952 in its side wall and/or on its distal end. Additionally, cannula 940 may have an optional cage arrangement 946 on its distal end 942.

However, in other embodiments no outlet tip 950 is used. Thus, there is only one inlet hole at distal end 942 of cannula 940. This single inlet hole may be surrounded by cage arrangement 946.

Cage arrangement 946 is one possible example. Other possible examples are described below with reference to FIGS. 16 to 20. Cage arrangement 946 may comprise for instance between 6 to 12 flexible wires, beams or bars. There may be for instance 8 cage wires 948 that span a sphere. The sphere and also the expelled blood prevent that the side wall of pulmonary artery PA covers one of outlet holes 952 of optional outlet tip 950. Furthermore, cage arrangement 946 fixes distal end 942 of cannula 940 within pulmonary artery PA.

Membrane 949 may cover only one half of cage arrangement 946, e.g. a half that is between the distal end of cannula 940 and the mid of cage wires 948. Examples of membranes that may be used on cage arrangement 946 are described below with reference to FIG. 16.

No extra care has to be taken because both cannulas 910 and 940 are inserted into veins in which there is comparably low blood pressure compared to the blood pressure in arteries. Antegrade infusion is performed into pulmonary artery PA that has many advantages because it corresponds to the natural direction of blood flow in lung L of the patient.

The arrangement shown in FIG. 9 may be used for patients with lung problems. Mobility of the patient is possible because no cannulas are used in femoral veins or arteries. The arrangement shown in FIG. 9 may be named pIVLP (percutaneous in vivo lung perfusion).

In other embodiments it is possible to insert cannula 910 through right internal jugular vein IJV/right subclavian vein to left atrium LA as described above and cannula 940 through left internal jugular vein IJV/left subclavian vein to left atrium LA.

Alternatively, device D9 may be a CO₂ (carbon dioxide) removal device, an oxygenator. Furthermore, the pumping direction may be reversed, i.e. from antegrade to retrograde.

During treatment of the lung L it is possible that the patient inhales a medicament or treatment substance in order to promote the treatment by the substance or medicament that flows through the vessels of the lung L and through the tissue of the alveoli. The fluid flow within circuitry 906 may comprise blood as a carrier substance. Alternatively other carrier substances may be used, for instance based on saline and/or on water.

FIG. 10 illustrates an extra corporeal retrograde lung perfusion circular blood flow circuitry 1006 comprising two dual lumen cannulas 1010 and 1040, two pumps P10a, P10b and an oxygenator device OXY10. Dual lumen cannula 1010 carries a cage arrangement 1016 near at least one inlet port that is arranged in pulmonary artery PA and an inlet portion 1090 that is arranged in right atrium RA. Second dual lumen cannula 1040 has at least one outlet port within ascending aorta aAO and an outlet portion 1084 in left atrium LA. The circuitry 1006 allows for instance the removal of thrombus from the lung L of patient. Alternatively, a chemotherapy of lung may be performed, a stem cell treatment or cleaning of the lung.

Dual lumen cannula 1010 is endovascularly inserted through right internal jugular vein IJV, superior vena cava SVC, right atrium RA, right ventricle RV, through pulmonary valve PVa into pulmonary artery PA. A guide wire (not shown) may be used to guide cannula 1010 to its final position. Alternatively, cannula 1010 may be inserted through the right subclavian vein and then along the same way as described above. Almost the whole blood that comes out of pulmonary artery PA is extracted into inner lumen of dual lumen cannula 1010, see arrow 1060, by using a membrane 1019 that is explained in more detail below. Other possibilities for insertion of a dual lumen cannula 1010 will be explained below, e.g. first insertion of outer lumen and then insertion of inner lumen.

Dual lumen cannula 1040 is endovascularly inserted through left internal jugular vein IJV, superior vena cava SVC, right atrium RA, trans-septal, i.e. through the septum between right atrium RA and left atrium LA, into left atrium LA. A guide wire (not shown) may be used to guide cannula 1040 to its final position. Alternatively, cannula 1040 may be inserted through the right subclavian vein. Almost the whole blood that exits the distal tip of the inner lumen of cannula 1040 is injected into ascending aorta aAO, see arrow 1070, by using a membrane 1049 that is explained in more detail below. Other possibilities for insertion of a dual lumen cannula 1040 will be explained below, e.g. first insertion of outer lumen and then insertion of inner lumen.

An optional inlet tip 1014 may be mounted on distal end 1012 of cannula 1010. Inlet tip 1014 may comprise a plurality of inlet holes 1015 in its side wall. Additionally, there may be a hole within the distal end of inlet tip 1014. The sum of the cross section areas of the holes of tip 1014 may be greater than the inner cross section area of cannula 1010 at its distal end 1012, for instance greater than twice the area or the triple of the area. This means that blood can be removed even if one or more of inlet holes 1015 in inlet tip 1014 is or are clogged.

However, in other embodiments no inlet tip 1014 is used. Thus, there is only one inlet hole at distal end 1012 of cannula 1010, i.e. at the proximal end of the cage arrangement. This single inlet hole would be surrounded by cage arrangement 1016. A single inlet hole may allow higher flow rates compared to inlet tip 1014 that comprises lateral inlet holes.

Cage arrangement 1016 is one possible example. Other possible examples are described below with reference to FIGS. 16 to 20. Cage arrangement 1016 may comprise for instance between 6 to 12 flexible wires, beams or bars. There may be for instance 8 cage wires 1018 that span a sphere. The sphere prevents that the side wall of left atrium LA covers one of inlet holes 1015 of tip 1014 or the single end hole if no inlet tip 1014 is used. Furthermore, cage arrangement 1016 fixes distal end 1012 of cannula 910 to pulmonary artery PA. Thus it is not possible that cannula 1010 slides back through pulmonary valve PVa into left ventricle LV.

Membrane 1019 may cover only one half of cage arrangement 1016, e.g. a half that is between distal end 1012 of cannula 1010 and the mid of cage wires 1018. Examples of membranes that may be used on cage arrangement 1016 are described below with reference to FIG. 16.

Inlet portion 1090 may comprise a plurality of inlet holes that extend through the side wall of outer lumen of cannula 1010. Blood is extracted by suction from right atrium RA into outer lumen of cannula 1010, preferably all blood or nearly all (for instance more than 90 percent of volume) blood that comes into right atrium RA, see arrow 1092. An optional cage arrangement may be arranged at inlet portion 1090.

Further to FIG. 10, a tube 1020a is connected to a proximal end of inner lumen of cannula 1010 and to an inlet of pump P10a. An outlet of pump P10a may be connected to a proximal end of outer lumen of cannula 1040 using a tube 1030a.

A tube 1020b is connected to a proximal end of outer lumen of cannula 1010 and to an inlet of pump P10b. An outlet of pump P10b may be connected to oxygenator device OXY10. A tube 1030b is connected to an outlet of oxygenator device OXY10 and to the proximal end of inner lumen of cannula 1040. It is also possible to exchange the sequence of pump P10b and oxygenator device OXY10.

Pumps P10a, P10b may be peristaltic pumps, centrifugal pumps, membrane pumps or other kind of pumps. Oxygenator device OXY10 enriches blood with oxygen that comes out of right atrium RA and/or right ventricle RV (see inlet portion 1098 that is described in more detail below) and is then injected into ascending aorta aAO. Thus, the function of the lung is fulfilled by oxygenator device OXY10 during treatment of lung L and the right heart H is supported.

Tubes 1020a, 1020b, 1030a, 1030b may be made of a flexible material or of a more rigid material. The circuitry 1006 may further include one or more blood filter units or units for dialysis of blood. Furthermore, a device may be used within circuitry 1006 for instance for injecting a drug or medicament and/or a treatment substance into the lung L of the patient.

Cannula 1040 may comprise an optional outlet tip 1050 that may have the same structure as inlet tip 1014 of cannula 1010. This means that outlet tip 1050 may comprise a plurality of outlet holes 1052 in its side wall and/or on its distal end. Additionally, cannula 1040 may have an optional cage arrangement 1046 on its distal end 1042. If there is no outlet tip 1050 a single end-hole may be used at the distal end of cannula 1040, i.e. at the proximal end of cage arrangement 1046.

Cage arrangement 1046 is one possible example. Other possible examples are described below with reference to FIGS. 16 to 20. Cage arrangement 1046 may comprise for instance between 6 to 12 flexible wires, beams or bars. There may be for instance 8 cage wires 1048 that span a sphere. The sphere and also the expelled blood prevent that the side wall of the ascending aorta aAO covers one of outlet holes 1052 of optional outlet tip 1050. The sphere prevents that the injected blood damages the walls of the aorta AO, i.e. the “sand blasting effect” is prevented or mitigated even if no outlet tip 1050 is used. Furthermore, cage arrangement 1046 fixes distal end 1042 of cannula 1040 within ascending aorta aAO.

Membrane 1049 may cover only one half of cage arrangement 1046, e.g. a half that is between the distal end of cannula 1040 and the mid of cage wires 1048. Examples of membranes that may be used on cage arrangement 1048 are described below with reference to FIG. 16.

Outlet portion 1084 of cannula 1040 may comprises a plurality of outlet holes 1085 that extend through the sidewall of the outer lumen of cannula 1040. Blood is expelled through outlet portion 1084 into the pairs of left and right pulmonary veins PV, see arrow 1075. Blood flow to left ventricle LV is thereby prevented by using membrane 1089. Outlet portion 1084 may be surrounded by an optional cage arrangement 1086. Moreover, the sand blasting effect is prevented or mitigated. Furthermore, cage arrangement 1086 fixes outlet portion 1086 of cannula 1040 within left atrium LA.

Cage arrangement 1086 is one possible example. Other possible examples are described below with reference to FIGS. 16 to 20. Cage arrangement 1086 may comprise for instance between 6 to 12 flexible wires, beams or bars. There may be for instance 8 cage wires 1088 that span a sphere. The sphere and also the expelled blood prevent that the side wall of left atrium LA covers one of outlet holes 1085. Furthermore, cage arrangement 1086 fixes outlet portion 1086 of cannula 1040 within left atrium LA.

Membrane 1089 may cover only one half of cage arrangement 1086, e.g. a half that is defined by two cage wires 1088 that are arranged opposite to each other or nearly opposite. Examples of membranes that may be used on cage arrangement 1086 are described below with reference to FIG. 17.

In summary, the following blood or other fluid flows are established within circuitry 1006:
a) from pulmonary artery PA through inner lumen of cannula 1010 via pump P10a through outer lumen of cannula 1040 to left atrium LA, i.e. lung perfusion, and
b) from right atrium RA and/or right ventricle RV through outer lumen of cannula 1010 via pump P10b and OXY10 through inner lumen of cannula 1040 to ascending aorta aAO, i.e. external enrichment of blood with oxygen.

Flow a) is closed via right and left pulmonary veins PV, tissue of the lungs and right and left pulmonary arteries, and pulmonary artery PA. Flow b) is closed via arteries of the body, for instance common femoral artery CFA, tissues of the body and the veins of the body, for instance common femoral vein CFV.

No extra care has to be taken because both cannulas 1010 and 1040 are inserted into veins in which there is comparably low blood pressure compared to blood pressure in arteries.

The arrangement shown in FIG. 10 may be used for patients with lung problems. Mobility of the patient is possible because no cannulas in femoral veins or arteries are used. The arrangement shown in FIG. 10 may be named pIVLP (percutaneous in vivo lung perfusion) retrograde.

In other embodiments it is possible to insert cannula 1010 through internal jugular vein IJV/left subclavian vein to pulmonary artery PA as described above and cannula 1040 through right internal jugular vein IJV/right subclavian vein to ascending aorta aAO.

An optional inlet portion 1098 may be arranged on a part of the outer lumen of cannula 1010 that is within right ventricle RV if cannula 1010 is put in place. Thus, it is possible to extract more blood from the right side of heart H using inlet portions 1090 and 1098 during retrograde lung perfusion. An optional cage arrangement may be arranged around inlet portion 1098.

During treatment of the lung L it is possible that the patient inhales a medicament or treatment substance in order to promote the treatment by the substance or medicament that flows through the vessels of the lung L and through the tissue of the alveoli. The fluid flow within the part of circuitry 1006 which comprises pump P10a may comprise blood as a carrier substance. Alternatively other carrier substances may be used, for instance based on saline and/or on water. The treatment substance may also be injected into the part of circuitry 1006 that comprises pump 10a. Furthermore, an adsorber/filter unit ADS and/or an oxygenator OXY and/or a carbon dioxide removal unit may be arranged within the part of circuitry 1006 that comprises pump 10a.

Furthermore, FIG. 10 illustrates an extra corporeal antegrade lung perfusion circular blood flow circuitry 1006 comprising two dual lumen cannulas 1010 and 1040, two pumps P10a, P10b and an oxygenator device OXY10. The proposed antegrade lung perfusion uses the arrangement 1006 of cannulas 1010 and 1040 as described above for retrograde lung perfusion. Reference is made to the description above in order to avoid unnecessary repetition. However, only the differences will be described. The main difference is that the direction of fluid flow in pump P10a is in the opposite direction now, see arrows 1094 and 1095. The direction of the blood flow or other fluid flow within the veins and arteries of the lung L is now antegrade.

Arrow 1096 shows that fluid is expelled from the distal end 1012 of cannula 1010 into pulmonary artery PA. Holes 1015 are outlet holes for antegrade lung perfusion and optional tip 1014 is an optional outlet tip antegrade lung perfusion. However, alternatively, a single end-hole may be used. Membrane 1014 directs fluid flow into pulmonary artery PA completely or almost completely. Furthermore, membrane 1019 may have a valve function allowing blood/fluid flow from right ventricle into pulmonary artery but not in the inverse direction.

Arrow 1097 shows that blood or other fluid that comes out of pulmonary veins PV is extracted by suction into outer lumen of cannula 1040. Holes 1085 are inlet holes for antegrade lung perfusion and portion 1084 is an inlet portion for antegrade lung perfusion. Membrane 1089 directs fluid flow from pulmonary veins PV completely or almost completely into outer lumen of cannula 1040.

In summary, the following flows are established for antegrade lung perfusions within circuitry 1006:
a) from left atrium LA through outer lumen of cannula 1040 via pump P10a through inner lumen of cannula 1010 to pulmonary artery PA, i.e. lung perfusion, and
b) from right atrium RA and/or right ventricle RV through outer lumen of cannula 1010 via pump P10b and OXY10 through inner lumen of cannula 1040 to ascending aorta aAO, i.e. external enrichment of blood with oxygen.

Flow a) is closed via pulmonary artery PA, right pulmonary artery rPA/left pulmonary artery lPA, tissue of the lung L and right/left pulmonary veins PV. Flow b) is closed via arteries of the body, for instance common femoral artery CFA, tissues of the body and the veins of the body, for instance common femoral vein CFV.

Even for antegrade lung perfusion, no extra care has to be taken because both cannulas 1010 and 1040 are inserted into veins in which blood pressure is comparably low compared to blood pressure in arteries.

The arrangement shown in FIG. 10 may be used for patients with lung problems. Mobility of the patient is possible because no cannulas in femoral veins or arteries are used. The arrangement for antegrade lung perfusion shown in FIG. 10 may be named pIVLP (percutaneous in vivo lung perfusion) antegrade.

Also for antegrade lung perfusion, it is possible to insert cannula 1010 through left internal jugular vein IJV/left subclavian vein to pulmonary artery PA as described above and cannula 1040 through right internal jugular vein IJV/right subclavian vein to ascending aorta aAO.

An optional inlet portion 1098 may be arranged on a part of the outer lumen of cannula 1010 that is within right ventricle RV if cannula 1010 is put in place. Thus, it is possible to extract more blood from the right side of heart H using inlet portions 1090 and 1098 during antegrade lung perfusion.

During treatment of the lung L it is possible that the patient inhales a medicament or treatment substance in order to promote the treatment by the substance or medicament that flows through the vessels of the lung L and through the tissue of the alveoli. The fluid flow within the part of circuitry 1006 which comprises pump P10a may comprise blood as a carrier substance. Alternatively other carrier substances may be used, for instance based on saline and/or on water. The treatment substance may also be injected into the part of circuitry 1006 that comprises pump 10a. Furthermore, an adsorber/filter unit ADS and/or an oxygenator OXY and/or a carbon dioxide removal unit may be arranged within the part of circuitry 1006 that comprises pump 10a.

Furthermore, it is possible to switch fluid flow direction between antegrade and retrograde, starting with antegrade fluid flow in lung L vessels or with retrograde fluid flow whichever is appropriate. Switching may be repeated during one treatment as often as necessary. Switching may ease the removal of at least one thrombus, especially of a blood thrombus.

Moreover, FIG. 10 illustrates an extra corporeal lobe dedicated antegrade lung perfusion circular blood flow circuitry 1006 comprising two dual lumen cannulas 1010 and 1040, two pumps P10a, P10b and an oxygenator device OXY10. The proposed lobe dedicated antegrade lung perfusion uses arrangement 1006 of cannulas 1010 and 1040 as described above for retrograde lung perfusion. Reference is made to the description above in order to avoid unnecessary repetition. However, only the differences will be described. One main difference is that the direction of blood/fluid flow in pump P10a is opposite to the direction mentioned above, see arrows 1094 and 1095. The other direction of fluid flow results in a change of the direction of the blood flow or other fluid flow within the veins and arteries of the lung.

A further difference is that cannula 1010 would have a longer portion between inlet portion 1090 and distal end 1012 enabling an arrangement of a cage arrangement 1016a within left pulmonary artery lPA as shown in FIG. 10. Cage arrangement 1016a is adapted to the diameter of left pulmonary artery lPA, i.e. it would be smaller than cage arrangement 1016. The other features of cage arrangement 1016a would be similar to the corresponding features of cage arrangement 1016 and would have an appropriate reduction in size, i.e. cage wires, membrane, optional outlet tip etc.

However, it is also possible to use an inflatable balloon instead of cage arrangement 1016a, see description of FIGS. 26 and 27 below.

The membrane of cage arrangement 1016a directs fluid flow that is expelled through inner lumen of cannula 1040 completely or almost completely into left pulmonary artery lPA. Furthermore, this membrane may have a valve function allowing blood flow from pulmonary artery PA also into left pulmonary artery lPA but not in the inverse direction.

Arrow 1097 shows that blood or other fluid that comes out of pulmonary veins PV is extracted by suction into outer lumen of cannula 1040 that is unchanged. Holes 1085 are inlet holes for antegrade lung perfusion and portion 1084 is an inlet portion for antegrade lung perfusion. Membrane 1089 directs fluid flow from pulmonary veins PV completely or almost completely into outer lumen of cannula 1040. Right pulmonary veins rPV will expel the fluid that is injected by cage arrangement 1016 and left pulmonary veins lPV will expel normal blood flow.

In summary, the following flows are established for dedicated antegrade lung perfusions within modified circuitry 1006:
a) from left atrium LA through outer lumen of cannula 1040 via pump P10a through inner lumen of cannula 1010 to pulmonary artery PA, i.e. lung perfusion, and
b) from right atrium RA and/or right ventricle RV through outer lumen of cannula 1010 via pump P10b and OXY10 through inner lumen of cannula 1040 to ascending aorta aAO, i.e. external enrichment of blood with oxygen.
c) normal blood flow from right ventricle RV through pulmonary artery PA, through right pulmonary artery rPA, tissue of lung L back via right pulmonary vein rPV into left atrium LA.

Flow a) is closed via left pulmonary artery lPA, tissue of left lung lobe of lung L and left pulmonary veins lPV. Flow b) is closed via arteries of the body, for instance common femoral artery CFA, tissues of the body and the veins of the body, for instance common femoral vein CFV.

Even for lobe dedicated antegrade lung perfusion, no extra care has to be taken because both cannulas 1010 and 1040 are inserted into veins in which there is comparably low blood pressure compared to blood pressure within arteries.

The modified arrangement shown in FIG. 10 may be used for patients with lung problems. Mobility of the patient is possible because no cannulas in femoral veins or arteries are used. The arrangement for dedicated lobe antegrade lung perfusion shown in FIG. 10 may be named pIVLP (percutaneous in vivo lung perfusion) antegrade lobe dedicated.

Also for dedicated lobe antegrade lung perfusion, it is possible to insert cannula 1010 through left internal jugular vein IJV/left subclavian vein to pulmonary artery PA as described above and cannula 1040 through right internal jugular vein IJV/right subclavian vein to ascending aorta aAO.

In another embodiment the right lobe of lung L may be flushed in the same way as described above for the left lobe of lung L. In this case, cage arrangement 1016a of cannula 1010 having a longer portion between inlet portion 1090 and distal end 1012 than shown in FIG. 10 would be arranged within right pulmonary artery rPA. Left pulmonary artery lPA would be filled with normal blood flow coming from right ventricle RV.

Furthermore, treatment of both lobes of lung L is possible is possible sequentially, e.g. treating the left lobe first and then the right lobe or vice versa. Several changes of the lobes that are treated are possible as well. The afterload that arises within heart H may be reduced in this way. Further positive effects may be possible as well. Detrimental effects may be limited to only one lobe. After a period of recreation the other lobe may be treated. Moreover, there may be disease that require only the treatment of one lobe of lung L, for instance a thrombus in only one of the lobes of lung L.

An optional inlet portion 1098 may be arranged on a part of the outer lumen of cannula 1010 that is within right ventricle RV if cannula 1010 is put in place. Thus, it is possible to extract more blood from the right side of heart H using inlet portions 1090 and 1098 during dedicated lobe antegrade lung perfusion.

During dedicated treatment of only one lobe of the lung L it is possible that the patient inhales a medicament or treatment substance in order to promote the treatment by the substance or medicament that flows through the vessels of the lung L and through the tissue of the alveoli. The fluid flow within the part of circuitry 1006 which comprises pump P10a may comprise blood as a carrier substance. Alternatively other carrier substances may be used, for instance based on saline and/or on water. The treatment substance may also be injected into the part of circuitry 1006 that comprises pump 10a. Furthermore, an adsorber/filter unit ADS and/or an oxygenator OXY and/or a carbon dioxide removal unit may be arranged within the part of circuitry 1006 that comprises pump 10a.

A dedicated retrograde treatment of the lobes of lung L seems feasible if further measures are taken, for instance usage of at least one split tip cannula, for instance within the left pulmonary veins lPV or within the right pulmonary veins rPV, preferably comprising at least two border elements, preferably expandable border elements, see FIGS. 26 and 27.

### D) RIGHT VENTRICLE ASSIST

FIG. 11 illustrates a right ventricle assist circuitry 1106 with one inlet stage or with multi inlet stages. Circuitry 1006 comprises one dual lumen cannula 1110 and one pump P11. Dual lumen cannula 1110 carries a cage arrangement 1146 near at least one outlet port that is arranged in pulmonary artery PA and at least one inlet portion 1190 that is arranged in right atrium RA. A second optional inlet portion 1198 may be arranged within right ventricle when cannula 1110 is arranged in place as shown in FIG. 11. It is also possible to use only inlet portion 1198 in right ventricle RV without having inlet portion 1190. Circuitry 1006 may be used for right ventricle assist.

Dual lumen cannula 1110 is inserted through left internal jugular vein IJV, superior vena cava SVC, right atrium RA, right ventricle RV, through pulmonary valve PVa into pulmonary artery PA. A guide wire (not shown) may be used to guide cannula 1110 to its final position. Alternatively, cannula 1010 may be inserted through the left subclavian vein and then along the same way as described above.

Almost the whole blood that comes out of inner lumen of cannula 1110 is injected into pulmonary artery PA, see arrow 1170, by using a membrane 1149 that is explained in more detail below. Other possibilities for insertion of a dual lumen cannula 1110 will be explained below, e.g. first insertion of outer lumen and then insertion of inner lumen.

Inlet portion 1190 may comprise a plurality of inlet holes that extend through the side wall of the outer lumen of cannula 1110. Blood is extracted by suction from right atrium RA into outer lumen of cannula 1110, preferably all blood or nearly all (for instance more than 90 percent of volume) blood that comes into right atrium RA, see arrow 1160. Optional inlet portion 1198 may comprise a plurality of inlet holes that extend through the side wall of the outer lumen of cannula 1110. Blood is extracted by suction from right ventricle RV into outer lumen of cannula 1110, preferably all blood or nearly all (for instance more than 90 percent of volume) blood that comes into left ventricle LV, see arrow 1199.

With reference further to FIG. 11, a tube 1120a is connected to a proximal end of outer lumen of cannula 1110 and to an inlet of pump P11. An outlet of pump P11 may be connected to a proximal end of inner lumen of cannula 1110 using a tube 1030. Pump P11 may be peristaltic pump or a centrifugal pump.

Tubes 1120, 1130 may be made of a flexible material or of a more rigid material. The circuitry 1106 may further include one or more blood filter units or units for dialysis of blood.

Cannula 1140 may comprise an optional outlet tip 1150. Outlet tip 1150 may comprise a plurality of outlet holes 1152 in its side wall and/or on its distal end. Additionally, cannula 1110 may have an optional cage arrangement 1146 on its distal end 1142.

Cage arrangement 1146 is one possible example. Other possible examples are described below with reference to FIGS. 16 to 20. Cage arrangement 1146 may comprise for instance between 6 to 12 flexible wires, beams or bars. There may be for instance 8 cage wires 1148 that span a sphere. The sphere and also the expelled blood prevent that the side wall of the pulmonary artery PA covers one of outlet holes 1152 of optional outlet tip 1150. Furthermore, cage arrangement 1146 fixes distal end 1142 of cannula 1140 within pulmonary artery PA.

Membrane 1149 covers only one half of cage arrangement 1146, e.g. a half that is between distal end 1142 of cannula 1140 and mid of cage wires 1148. Examples of membranes that may be used on cage arrangement 1148 are described below with reference to FIG. 16. Membrane 1149 directs blood into pulmonary artery PA and prevents that blood flows back into right ventricle RV. Membrane 1178 may have a valve function, e.g. if remaining blood comes from right ventricle RV it can pass between membrane 1149 and sidewalls of pulmonary artery PA.

The following blood flow that is established within circuitry 1106 is from right atrium RA and/or right ventricle RV through outer lumen of cannula 1110 via pump P11 back through inner lumen of cannula 1110 to pulmonary artery. This flow is closed via right and left pulmonary veins PV, arteries of body 100, for instance common femoral artery CFA, tissue of body 100 and veins of body 100, for instance common femoral vein CFV.

The arrangement shown in FIG. 11 may be used for patients without lung problems. Mobility of the patient is possible because no cannulas in femoral veins or arteries are used. The arrangement for shown in FIG. 11 may be named pRVAD (percutaneous right ventricle assist device) multi lumen.

It is possible to insert cannula 1110 through right internal jugular vein IJV/right subclavian vein to pulmonary artery PA as described above.

Other applications of the proposed cage arrangements and/or dual lumen cannulas than these shown in FIGS. 1 to 11 are possible as well. All cannulas shown may be used with or without cages.

The cannula systems CS1 to CS3 that are described with reference to FIGS. 12 to 14 are further examples for dual lumen cannula systems shown in FIGS. 2, 3, 10 and 11. FIG. 12 illustrates a cannula system CS1 having an inner cannula I1 and an outer cannula O1 that are arranged coaxially relative to each other with the inner cannula I1 arranged inside the outer cannula O1. Outer cannula O1 is named as a first cannula in the claims. Inner cannula I1 is named as a second cannula in the claims. The outer cannula/first cannula O1 to O3 may have a minimum inner diameter or width. The inner cannula/second cannula I1 to I3 may have a maximum outer diameter or width, wherein the minimum inner diameter or width of the outer cannula/first cannula O1 to O3 is greater than the maximum outer diameter or width inner cannula/second cannula I1 to I3.

Outer cannula O1 is or may be inserted into body 100 first, i.e. preferably before the inner cannula I1 will be inserted. Only after the insertion of the outer cannula O1 into body 100, preferably after the insertion of outer cannula O1 is completed, i.e. the outer cannula O1 has reached its destination position, inner cannula I1 is inserted into outer cannula O1 and then further beyond the distal end of outer cannula O1.

Both cannulas O1 and I1 are bendable up to a specific degree, i.e. they are bendable in radial directions. However, the diameter of cannulas O1 and I1 may not be variable in the sense that the area of the diameter cross section may be increased or decreased essentially.

Outer cannula O1 may have a circular or oval cross section along its entire length. A port P1a of outer cannula O1 may be arranged at a proximal P end of a sidewall of outer cannula O1. The proximal P end of outer cannula O1 may comprise a proximal surface, for instance a flat surface, that may have an opening OP1. Opening OP1 may be arranged on the longitudinal axis A of outer cannula O1.

Inner cannula I1 may also have a circular or oval cross section along its entire length. A port P1b of inner cannula I1 may be arranged at a proximal P end of inner cannula I1. Inner cannula I1 may be inserted through opening OP1 into outer cannula O1. Thereby, the inner cannula I1 may be arranged on the longitudinal axis A of outer cannula O1. At least one mounting portion MP1 or mounting elements may be arranged on an outer surface of inner cannula I1, e.g. protruding radially outward, and/or on an inner surface of outer cannula O1, e.g. protruding radially inward. Mounting portions MP1 may center inner cannula I1 within outer cannula O1.

A sealing element S1 may be used to seal cannula system CS1 proximally. Sealing element S1 may be arranged within opening OP1 or at another appropriate location. Sealing element S1 may be an O-ring in the simplest case. Alternatively, a multi-flap valve or a membrane may be used.

An optional fixation element FE1 may be arranged completely outside of outer cannula O1. Fixation element FE1 may have a first state in which axial movement M1 of inner cannula I1 relative to outer cannula O1 is possible or allowed and a second state that blocks such axial movement. Fixation element FE1 may operate automatically or semi-automatically or may be operated manually. Thus, fixation element FE1 may block axial movement if a predetermined length of inner cannula I1 is introduced into outer cannula O2. Alternatively, blocking may be performed manually at several positions of inner cannula I1 within outer cannula O1. It may be possible to bring fixation element FE1 back to the first state after it is in the blocking state.

Alternatively, fixation element FE1 may be arranged partly or completely within outer cannula O1. If it is completely within outer cannula O1 manual access to fixation element FE1 may be possible by operating elements. Alternatively, no manual access may be possible, i.e. fixation element FE1 may be operated in an automatic or semi-automatic mode depending for instance on the overlapping length of both cannulas I1 and O1.

FIG. 13 illustrates a cannula system CS2 having an inner (second) cannula I2 that is arranged loosely within an outer (first) cannula O2. Outer cannula O2 is named as a first cannula in the claims. Inner cannula I2 is named as a second cannula in the claims.

Outer cannula O2 is inserted into body 100 first, i.e. preferably before the inner cannula I2 will be inserted. Only after the insertion of the outer cannula O2 into body 100, preferably after the insertion of outer cannula O2 is completed, i.e. the outer cannula O2 has reached its destination position, inner cannula I2 is inserted into outer cannula O2 and then further beyond the distal end of outer cannula O2.

Both cannulas O2 and I2 are bendable up to a specific degree, i.e. they are bendable in radial directions. However, the diameter of cannulas O2 and I2 may not be variable in the sense that the area of the diameter cross section may be increased or decreased essentially.

Outer cannula O2 may have a circular or oval cross section along its entire length. A port P2a of outer cannula O2 may be arranged at a proximal P end of outer cannula O2 that may be arranged on the longitudinal axis of outer cannula O2. The sidewall of outer cannula O2 may have an opening OP2 at its proximal P end. Opening OP2 may face laterally and or transversally relative to longitudinal axis A of outer cannula O2.

Inner cannula I2 may also have a circular or oval cross section along its entire length. A port P2b of inner cannula I2 may be arranged at a proximal P end of inner cannula I1. Inner cannula I2 may be inserted through opening OP2 into outer cannula O2. Thereby, the inner cannula I1 may be arranged loosely radially to longitudinal axis A of outer cannula O1. A mounting portion may not be necessary.

A sealing element S2 may be used to seal cannula system CS2 proximally. Sealing element S2 may be arranged within opening OP2 or at another appropriate location. Sealing element S2 may be an O-ring in the simplest case. Alternatively, a multi-flap valve or a membrane may be used.

An optional fixation element FE2 may be arranged completely outside of outer cannula O2. Fixation element FE2 may have a first state in which an axial movement M2 of inner cannula I2 relative to outer cannula O2 is possible or allowed and a second state that blocks such axial movement. Fixation element FE2 may operate automatically or semi-automatically or may be operated manually. Thus fixation element FE2 may block axial movement if a predetermined length of inner cannula I2 is introduced or inserted into outer cannula O2. Alternatively, blocking may be performed manually at several positions of inner cannula I2 within outer cannula O2. It may be possible to bring fixation element FE2 back to the first state after it is in the blocking state.

Alternatively, fixation element FE2 may be arranged partly or completely within outer cannula O2. If it is completely within outer cannula O2 manual access to fixation element FE2 may be possible by operating elements. Alternatively, no manual access may be possible, i.e. fixation element FE2 may be operated in an automatic or semi-automatic mode depending for instance on the overlapping length of both cannulas I2 and O2.

FIG. 14 illustrates a cross section of another cannula system CS3 that comprises an outer cannula O3 and an inner cannula I3. Outer cannula O3 is named as a first cannula in the claims. Inner cannula I3 is named as a second cannula in the claims.

Outer cannula O3 has a circular inner cross section, preferably along its whole length. Alternatively, outer cannula O3 may have an oval or elliptic inner cross section, preferably along its whole length.

Inner cannula I3 has an outer cross section that is complementary to the inner cross section of outer cannula O3 and that leaves a lumen (first lumen in the claims) for the transport a fluid through outer cannula O3. If outer cannula O3 has an oval inner cross section, the outer cross section of inner cannula may be also oval or elliptic minus a part that is used for fluid transport in outer cannula O3.

The fluid may be blood or may comprise blood, for instance blood enhanced with a medicament or drug. Alternatively other fluids than blood may be used.

Inner cannula I3 may have a flat outer surface that is arranged for instance along the longitudinal axis A of outer cannula O3. Alternatively, this flat surface of inner cannula I3 may be arranged on a side of the longitudinal axis A of outer cannula O3 on which the first lumen of the outer cannula O3 for fluid transport is located, see line L3. In a further alternative, the flat surface of inner cannula I3 may be arranged on a side of the longitudinal axis A of outer cannula O3 that is opposite to the side that comprises the main part of the first lumen of the outer cannula O3 for fluid transport, see line L4.

No mounting elements are necessary in cannula system CS3. However, it is possible to use mounting elements that position or fix the inner cannula I3 radially relative to outer cannula O3. Positioning would be easier than in cannula system CS1 because the complementary shapes of inner cannula I3 and outer cannula O3 may be used to enhance a specific positioning of inner cannula I3 within outer cannula O3.

FIG. 15 illustrates an embodiment of a dual lumen cannula system 1500 comprising at least one pre bended outer cannula 1508 and an inner cannula that is not shown. Cannula system 1500 is adapted for a stepwise insertion of the cannula 1508 and of the other cannula, i.e. first outer cannula 1508 and then inner cannula. Dual lumen system 1500 may comprise:

a locking mechanism 1502 that may lock an introducer that is used for introducing outer cannula 1508. FIG. 21 shows an example in which an introducer is inserted into a cannula system. The introducer may be locked by force fitting or by another appropriate mechanism.

an adapter portion 1504 that may be used to connect locking mechanism removably to cannula system 1500,

a handle portion 1506 that may also be connected removably to cannula system 1500 and that is used to ease introducing of outer cannula 1508 or inner cannula into body 100 of a patient. Handle portion 1506 may be compressible by a medical clamp or by forceps.

There may be a pre-bended kink K or bend that is between a long straight portion of cannula 1508 of system 1500 and a shorter straight portion. Cannula 1508 may have a straight insertable length L10 up to kink K and a short straight portion of cannula system 1500 having an insertable length L20 between kink K and the distal end. Kink K may also be positioned on other positions than the position shown in FIG. 15, e.g. more distally D or more proximally P. Cannula system 1500, especially cannula 1508, is or may be flexible, i.e. it is possible to bend each portion and of course to bring the whole cannula system 1500 in a straight shape. However, without external forces, kink K will bring cannula 1508 of system 1500 in the shape that is shown in FIG. 15 again. The inner cannula of cannula system 1500 may not have a kink K but may be straight. Alternatively, the inner cannula of cannula system 1500 may have a kink K. The cannula comprising or having the bend may be used for endovascular jugular insertion into the left atrium or into the pulmonary artery.

Although, cannula 1508 is shown having no diameter variable arrangement DVA or cage arrangement on the tip there may be such a diameter variable arrangement DVA1. Cannula 1508 may comprise only one end-hole that may be closed by a closure element that allows passage of the inner cannula but not of blood into cannula 1508 through the end-hole or vice versa. The lateral or side holes of cannula 1508 may be placed within the right atrium RA of the heart H whereas the cage arrangement may be placed within the left atrium LA of the heart H. Insertion of the inner cannula may further be promoted if at least one wire of diameter variable arrangement DVA1 is omitted.

In an alternative embodiment a cage arrangement or diameter variable arrangement DVA2 is used and the distal tip with lateral holes is omitted. There may be a membrane connected to diameter variable arrangement DVA2 that has an opening which faces laterally, see cage arrangement 1086 in FIG. 10 and in the variants of FIG. 10. Cannula 1508 may have only one end-hole in this case, preferably an end-hole that is not closed by a valve and/or membrane. Introduction of the inner cannula of cannula system 1500 may be easier compared to the case in which a distal tip having lateral holes is include within diameter variable arrangement DVA2. However, in a further alternative, a distal tip with lateral holes and/or with an end hole may be used. Insertion of the inner cannula may further be promoted if at least one wire of diameter variable arrangement DVA2 is omitted.

Cannula 1508 may be used as a delivery cannula or as a drainage cannula.

Cannula system 1500 may be used for jugular access to heart H or for other purposes. Length L10 refers to the length from the proximal end of handle portion 1506 to the pre bended kink K, i.e. the length of the longer straight portion of cannula 1508. An example for length L10 is 300 mm. Other values for length L10 are also possible.

Length L20 is the length of the pre bended distal portion, i.e. measured from the pre bended kink K to the distal end of cannula 1508, and without the length of a diameter variable arrangement if present. An example for length L20 may be for instance 70 mm (millimeter). Other values for length L20 are possible as well. Preferred values for length L20 are within the range of 3 cm to 7 cm.

An angle W1 between the two straight portions of cannula system 1500 at kink K may have a value of 130 degrees. However, a value within the range of 70 degrees to 145 degrees is also possible.

Cannula system 1500 may be used for left or right jugular or for left or right subclavian access to heart H or for other purposes. Longer cannulas are necessary for left side access and or for femoral access to the heart H. Modifications may be made with regard to length L10 and or length L20. Furthermore, the kink K may be at another position and angle W1 may have another value. It may also be useful to have a second pre-bended kink.

For cannula 1508 of cannula system 1500 the following table may be valid:

| size of cannula in F/ | SL1 | SL2 | L10 + L20 |
|---|---|---|---|
| overall length in cm | in mm | in mm | in cm |
| 21 F/32 | 7.0 | 2.3 | 32 |
| 21 F/42 | 7.0 | 2.3 | 42 |
| 21 F/62 | 7.0 | 2.3 | 62 |
| 21 F/72 | 7.0 | 2.3 | 72 |
| 31 F/42 | 10.3 | 2.3 | 42 |

There may be further intermediate sizes of cannulas having for instance an outer diameter of 23 F (French), 25 F, 27 F and 29 F combined with an overall length L10 plus L20 of for instance 32 cm, 42 cm or 62 cm. The overall length L10 plus L20 is the insertable length of cannula 1408.

SL1 is the outer diameter of outer or first cannula 1508 in French. SL2 is the diameter of lateral holes in the distal tip. The numbers given in the table or given above may vary within a range of minus 10 percent to plus 10 percent. Other sizes of the cannula system 1500 are possible as well.

The tip of inner cannula of cannula system that is shown in FIG. 15 may be optional. Furthermore, a cage arrangement (diameter variable arrangement) may be mounted on distal end of inner cannula. Additionally or alternatively, a cage arrangement (diameter variable arrangement) may be mounted on distal end of outer cannula 1508. None, one or both cage arrangements (diameter variable arrangement) may be covered by a respective membrane. The membrane of outer cannula 1508 may have an opening that faces distally or laterally or proximally. The membrane of the inner cannula may have an opening that faces distally, see for instance cannula 1040 in FIG. 10.

FIGS. 16 to 21 show embodiments of cage arrangements that may be used in all of the embodiments shown in FIGS. 1 to 11. FIG. 16 illustrates a cage arrangement 1600 comprising a membrane 1650 having an opening 1652 that faces distally. The shape of cage arrangement 1600 in its expanded state may be similar to a sphere or to a ball. Alternatively, an ellipsoid or another shape may be used. Cage arrangement 1600 is mounted to a cannula 1602 that may be an outer cannula or an inner cannula of a cannula system, for instance of one of cannula systems CS1 to CS3.

Cannula 1602 may comprise an optional cannula tip 1604 having apertures 1606, 1608 arranged in the pattern that is shown. However, other arrangements of apertures 1606, 1606 may be used, especially comprising a different number of apertures 1606, 1606 is possible. If cannula tip 1604 is not used there may be a single end-hole at the distal tip of cannula 1602. Cannula 1602 may not extend or may only extend by less than 10 mm into cage arrangement 1600.

If cage arrangement 1600 is viewed from above, it comprises in a counter clock wise direction eight cage wires 1610, 1612, etc. to 1624. More or less cage wires 1610 to 1624 may also be used. Cage wire 1624 is at the rear side of cage arrangement 1600. Cage wires 1610 to 1624 have, at a given axial position, same distances, especially same angularly distances, to the neighboring cage wires 1610 to 1624. One of the cage wires 1610 to 1624 or some of the cage wires 1610 to 1624 may be omitted, for instance to allow the insertion of further cannulas and/or cage arrangements through cage arrangement 1600.

Cage arrangement 1600 may have the following portions with increasing distance from mounting portion 1630:

a mounting portion 1630 at which the cage wires 1610 to 1624 are wound around distal end of cannula 1602, for instance at least three quarters of the circumference. Mounting portion 1630 may alternatively comprise an additional mounting element on which cage wires 1610 to 1624 are mounted, for instance a mounting sleeve or a jacket. In both cases a circumferential notch in cannula 1602 may be used to prevent axial movement of cage arrangement 1600 relative to cannula 1602. Additional or alternative mounting techniques may be used, i.e. welding, soldering, glue etc.

a proximal portion 1631 in which neighboring cage wires have increasing distances with regard to each other and with increasing distance to mounting portion 1630,

a comparably short optional transition portion 1632 in which cage wires 1610 to 1624 are arranged essentially parallel relative to each other and/or the longitudinal axis of cannula 1602 as well as to the longitudinal axis of cage arrangement 1600,

a distal portion 1633 in which neighboring cage wires have decreasing distances with regard to each other and with increasing distance to mounting portion 1630, and

a cage tip portion 1635 in which the cage wires 1610 to 1624 are connected together, for instance by a plastic cap. Within tip portion 1635 cage wires 1610 to 1624 may be twisted or be arranged parallel with regard to each other.

All cage wires 1610 to 1624 may be pre-bended in the same way and/or may have the same shape memorized within the shape memory of the material of the cage wires 1610 to 1624. An example is given for cage wire 1612 that is arranged mainly within a plane that is equal to the plane of the sheet that shows FIG. 16. Cage wire 1612 comprises portions that correspond to portions 1630 to 1635 of cage arrangement 1600:

a mounting portion 1640 that comprises:
an optional circumferential portion 1638 in which the wire is shaped circular, for instance along at least three quarters of the circumference of a circle, and
an optional straight portion 1639 that may be arranged parallel to the longitudinal axis of cage arrangement 1600 and that may serve as a reference axis in the following—alternatively the longitudinal axis of cannula 1602 may be uses as a reference axis,

a proximal portion 1641 in which wire 1612 has an increasing radial distance to the reference axis with increasing distance to mounting portion 1640,

an optional transition portion 1642 in which wire 1612 has a constant radial distance to the reference axis with increasing distance to mounting portion 1640,

a distal portion 1643 in which wire 1612 has a decreasing radial distance to the reference axis with increasing distance to mounting portion 1640, and

a cage tip portion 1645 that may be covered by plastic cap and/or in which the wire 1612 is parallel to the reference axis or is spirally and/or helically wounded.

Membrane 1650 extends circumferential from proximal P end almost up to distal D end of cage arrangement 1600, i.e. portions 1631 and 1632 are covered completely and portion 1633 is covered at more than half of its axial length. An opening 1652 faces distally to distal cage tip 1654 relative to longitudinal axis of cannula 1602 or of cage arrangement 1600.

Membrane 1650 may cover only or at least the lower half or only or at least the upper half of cage arrangement 1600, see line. In the latter case the opening of membrane 1650 would be facing proximally. Membrane 1650 may cover also only the lower quarter or the lower three quarters of cage arrangement 1600. Reference may be made thereby to the axial length of cage arrangement 1600. Further, membrane 1650 may cover also only the upper quarter or the upper three quarters of cage arrangement 1600. However, cage arrangement 1600 may also be used without membrane 1600.

A separate spirally wounded wire that forms a coil may be used to form a mounting portion that is similar to mounting portion 1630. The number of windings within the coil may be in the range of 3 windings to 15 windings and/or in the range of 3 windings to 10 windings. There may be no space between adjacent or neighboring windings. Alternatively, there may be a small space between adjacent windings. The wires may have or may not have the circumferential portions, for instance 1638. The straight portions, for instance 1639, may be connected to the coil with the same axial position or with the same axial offset between angularly neighboring/adjacent wires, i.e. comparably to the arrangement that is shown in FIG. 16.

A sleeve may be used to form a mounting portion. The wires may have or may not have the circumferential portions, for instance 1638. The straight portions, for instance 1639, may be connected to the sleeve with the same axial position or with the same axial offset between angularly neighboring/adjacent wires, i.e. comparably to the arrangement that is shown in FIG. 16. The sleeve may be an inner sleeve relative to the wires or an outer sleeve. Furthermore, it is possible to use an inner sleeve and an outer sleeve with portions of the wires arranged therein between, especially straight portions that may be arranged in parallel or oblique to a longitudinal axis of the sleeve and/or of the cannula.

Other possibilities of the connection of the cage arrangement to the cannula may be used as well.

FIG. 17 illustrates a cage arrangement 1700 comprising a membrane 1750 having an opening that faces laterally. However, cage arrangement 1700 corresponds to cage arrangement 1600 except of the placement of membrane 1750. In order to avoid repetition reference is made to the description of FIG. 16 above. There are the following corresponding parts:

cage arrangement 1600, 1700,

cannula 1602, 1702,

optional cannula tip 1702, 1704,

apertures 1606, 1608, 1706, 1708,

cage wires 1610 to 1624, 1710 to 1724,

cage portions 1630 to 1635 are also valid for cage arrangement 1700,

circumferential portion 1638, 1738,

straight portions 1639, 1739,

cage wire portions 1640 to 1645 are also valid for cage arrangement 1700, and

cage tip 1654, 1754.

Features that are mentioned above for parts 1600 to 1645 apply also to the corresponding parts 1700 to 1739 and to the corresponding parts that are not indicated by reference signs in FIG. 17.

Membrane 1750 covers slightly more than half of cage arrangement 1700 and has an opening 1752 that faces laterally or transversally relative to the longitudinal axis of cannula 1702 or of cage arrangement 1700. Thus, in the expanded state of cage arrangement 1700, membrane 1750 is arranged between cage wires 1718, 1720; 1720, 1722; 1722, 1724; 1724, 1710 and 1710, 1712. Membrane 1750 may extend through all main portions of cage arrangement 1700 between these cage wires, i.e. proximal portion, optional transition portion and distal portion, see corresponding portions 1631 to 1633 in FIG. 16.

Other arrangements of membrane 1750 are possible as well each having an opening that faces laterally:

less than 90 degrees in circumferential direction, preferably more than 10 degrees or more than 45 degrees,

90 degrees or more than 90 degrees of coverage in circumferential direction, but preferably less than 110 degrees, less than 135 degrees or less than 180 degrees,

180 degrees of coverage, i.e. membrane 1750 is only arranged between cage wires 1720 to 1724 and further between cage wire 1724 and 1710 as well as between cage wire 1710 and 1712, alternatively there may be at least 180 degrees of coverage, or

270 degrees of coverage, i.e. membrane 1750 is arranged additionally between cage wires 1716 and 1718, alternatively there may be at least 270 degrees of coverage, or.

Other angles of coverage for membrane 1750 may be easily realized if more or less than eight cage wires 1710 to 1724 are used in cage arrangement 1700. However, cage arrangement 1700 may also be used without a membrane. Combinations of lateral and distal/proximal facing openings are also possible.

A separate spirally wounded wire that forms a coil may be used to form a mounting portion that is similar to mounting portion 1730. The number of windings within the coil may be in the range of 3 windings to 15 windings and/or in the range of 3 windings to 10 windings. There may be no space between adjacent or neighboring windings. Alternatively, there may be a small space between adjacent windings. The wires may have or may not have the circumferential portions, for instance 1738. The straight portions, for instance 1739, may be connected to the coil with the same axial position or with the same axial offset between angularly neighboring/adjacent wires, i.e. comparably to the arrangement that is shown in FIG. 17.

A sleeve may be used to form a mounting portion. The wires may have or may not have the circumferential portions, for instance 1738. The straight portions, for instance 1739, may be connected to the sleeve with the same axial position or with the same axial offset between angularly neighboring/adjacent wires, i.e. comparably to the arrangement that is shown in FIG. 17. The sleeve may be an inner sleeve relative to the wires or an outer sleeve. Furthermore, it is possible to use an inner sleeve and an outer sleeve with portions of the wires arranged therein between, especially straight portions that may be arranged in parallel or oblique to a longitudinal axis of the sleeve and/or of the cannula.

Other possibilities of the connection of the cage arrangement to the cannula may be used as well.

FIG. 18 illustrates a cage arrangement 1800 comprising a portion that is bended backwards, i.e. backwards bended portion 1834. The shape of cage arrangement 1800 in its expanded state may be similar to a sphere or to a ball. Alternatively, an ellipsoid or another shape may be used. Cage arrangement 1800 is mounted to a cannula 1802 that may be an outer cannula or an inner cannula of a cannula system, for instance of one of cannula systems CS1 to CS3.

Cannula 1802 may comprise an optional cannula tip 1804 having apertures 1806, 1808 arranged in the pattern that is shown. However, other arrangement of apertures 1806, 1806 may be used, especially comprising a different number of apertures 1806, 1806 is possible. If cannula tip 1804 is not used, there may be a single end-hole at the distal tip of cannula 1802. Cannula 1802 may not extend or may only extend by less than 10 mm into cage arrangement 1800.

If cage arrangement 1800 is viewed from above, it comprises in a counter clock wise direction eight cage wires 1810, 1812, etc. to 1824. More or less cage wires 1810 to 1824 may also be used. Cage wire 1810 to 1824 is at the rear side of cage arrangement 1600. Cage wires 1810 to 1824 have at a given axial position same distances to the neighboring cage wires 1810 to 1824. One of the cage wires 1810 to 1824 or some of the cage wires 1810 to 1824 may be omitted, for instance to allow the insertion of further cannulas and/or cage arrangements through cage arrangement 1800.

Cage arrangement 1800 may have the following portions with increasing distance from mounting portion 1830:

a mounting portion 1830 at which the cage wires 1810 to 1824 are wound around distal end of cannula 1802, for instance at least three quarters of the circumference. Mounting portion 1830 may alternatively comprise an additional mounting element on which cage wires 1810 to 1824 are mounted, for instance a mounting sleeve or a jacket. In both cases a circumferential notch in cannula 1802 may be used to prevent axial movement of cage arrangement 1800 relative to cannula 1802. Additional or alternative mounting techniques may be used, i.e. welding, soldering, glue etc.

a proximal portion 1831 in which neighboring cage wires have increasing distances with regard to each other and with increasing distance to mounting portion 1830,

a comparably short optional transition portion 1832 in which cage wires 1810 to 1824 are arranged essentially parallel relative to each other and/or the longitudinal axis of cannula 1802 as well as to the longitudinal axis of cage arrangement 1800, and

a distal portion 1833 in which neighboring cage wires have decreasing distances with regard to each other and with increasing distance to mounting portion 1830.

There may be a short optional radial portion that forms a plane for contact with a wall of a vessel or a chamber of the heart. The radial length of this radial portion may be in the range of 3 mm to 10 mm (millimeters). In the expanded state, wire portions within the radial portion extend only radially but not axially, i.e. the wire portions have the same axial position and extend to the extended longitudinal axis of cannula 1802.

Furthermore, cage arrangement 1800 may comprise following the distal portion 1833:

a backwards bended portion 1834 in which cage wires 1810 to 1824 change direction and in which neighboring cage wires 1810 to 1824 have decreasing distances with regard to each other and with decreasing distance to mounting portion 1830, and

a cage tip portion 1835 in which the cage wires 1810 to 1824 are connected together, for instance by a plastic cap. Within tip portion 1835 cage wires 1810 to 1824 may be twisted or be arranged parallel with regard to each other.

All cage wires 1810 to 1824 may be pre-bended in the same way and/or may have the same shape memorized within the shape memory of the material of the cage wires 1810 to 1824. An example is given for cage wire 1812 that is arranged mainly within a plane that is equal to the plane of the sheet that shows FIG. 18. Cage wire 1812 comprises portions that correspond to portions 1830 to 1835 of cage arrangement 1800:

a mounting portion 1840 that comprises:
an optional circumferential portion 1838 in which the wire is shaped circular, for instance along at least three quarters of the circumference of a circle, and
an optional straight portion 1839 that may be arranged parallel to the longitudinal axis of cage arrangement 1800 and that may serve as a reference axis in the following—alternatively the longitudinal axis of cannula 1802 may be uses as a reference axis,

a proximal portion 1841 in which wire 1812 has an increasing radial distance to the reference axis with increasing distance to mounting portion 1840,

an optional transition portion 1842 in which wire 1812 has a constant radial distance to the reference axis with increasing distance to mounting portion 1840, and

a distal portion 1843 in which wire 1812 has a decreasing radial distance to the reference axis with increasing distance to mounting portion 1840.

There may be the optional radial portion that is mentioned above. The optional radial portion may be arranged between the distal portion 1843 and the backwardly bended wire portion 1844.

Furthermore cage wire 1812 may comprise following the distal portion 1843:

a backwardly bended wire portion 1844, and

a cage tip portion 1845 that may be covered by plastic cap and/or in which the wire 1812 is parallel to the reference axis or is spirally and/or helically wounded.

In another embodiment cage arrangement 1800 may be covered at least partially by a membrane. The membrane may have an opening that faces distally or proximally, see description of FIG. 16, for instance coverage of lower three quarters. Alternatively, the membrane may have an opening that faces laterally, see description of FIG. 17, for instance coverage of at least half of the circumference or of three quarter of circumference. Combinations of lateral and distal/proximal facing openings are also possible.

Other shapes of cage arrangements with a backward bended portion are also possible, see for instance shapes similar to the shapes that are shown in FIGS. 19 and 20, e.g. cone shape or cylinder shape.

A separate spirally wounded wire that forms a coil may be used to form a mounting portion that is similar to mounting portion 1830. The number of windings within the coil may be in the range of 3 windings to 15 windings and/or in the range of 3 windings to 10 windings. There may be no space between adjacent or neighboring windings. Alternatively, there may be a small space between adjacent windings. The wires may have or may not have the circumferential portions, for instance 1838. The straight portions, for instance 1839, may be connected to the coil with the same axial position or with the same axial offset between angularly neighboring/adjacent wires, i.e. comparably to the arrangement that is shown in FIG. 18.

A sleeve may be used to form a mounting portion. The wires may have or may not have the circumferential portions, for instance 1838. The straight portions, for instance 1839, may be connected to the sleeve with the same axial position or with the same axial offset between angularly neighboring/adjacent wires, i.e. comparably to the arrangement that is shown in FIG. 18. The sleeve may be an inner sleeve relative to the wires or an outer sleeve. Furthermore, it is possible to use an inner sleeve and an outer sleeve with portions of the wires arranged therein between, especially straight portions that are arranged in parallel or oblique to a longitudinal axis of the sleeve and/or of the cannula.

Other possibilities of the connection of the cage arrangement to the cannula may be used as well.

FIG. 19 illustrates cannulas 1908, 1910 of a cannula system 1900. Cannula 1908 is an outer cannula 1908 of cannula system 1900. Cannula 1910 is an inner cannula 1910 of cannula system 1900. The length and/or outer diameters of cannulas 1908 and 1910 may be different, i.e. inner cannula 1910 may be longer and thinner than outer cannula 1908. Disregarding these differences, both cannulas 1908 and 1910 may correspond to the picture that is shown in FIG. 19. One of the cannulas 1908, 1910 or both cannulas 1908, 1910 may comprise a cage arrangement 1912.

Cannula system 1900 may be adapted for a stepwise insertion of the cannulas 1908 and 1910, i.e. first outer cannula 1908 and then inner cannula 1910. Dual lumen cannula system 1900 may comprise, preferably as separate systems for each cannula 1908 and 1910 or only one system for both cannulas 1908 and 1910:

a locking mechanism 1902 that may lock an introducer or introducer member that is used for introducing outer cannula 1908 or inner cannula 1910, especially after outer cannula 1908 has been introduced. FIG. 21 shows an example in which an introducer is inserted into a cannula system. The introducer may be locked by force fitting or by another appropriate mechanism.

an adapter portion 1904 that may be used to connect locking mechanism 1902 removably to cannula system 1900, and

a handle portion 1906 that may also be connected removably to cannula system 1900 and that is used to ease introducing of outer cannula 1908 or inner cannula 1910 into body 100 of a patient.

Inner cannula 1908 and/or outer cannula 1910 may comprise a cage arrangement 1912 having wires that are essentially arranged in parallel with regard to each other in the main portion of the cage arrangement 1912, e.g. along the entire axial length of cage arrangement 1912 or along at least 90 percent of this length. Thus cage arrangement 1912 has the shape of a cylinder.

Cage arrangement 1912 of inner cannula 1908 and/or outer cannula 1910 may have or may comprise a membrane, for instance a membrane that has an opening facing distally or laterally, see for instance FIG. 16 and FIG. 17 and corresponding descriptions.

Cannula 1908 and/or cannula 1910 may be pre-bended as described above for cannula system 1900. The tip of cannula 1908 and/or 1909 may be optional, i.e. there may be only one end-hole opening within cage arrangement 1912, for instance at its proximal end.

Cage arrangement 1912 may be used also for a single lumen cannula, preferably with or without a membrane.

FIG. 20 illustrates cannulas 2008, 2010 of a cannula system 2000 comprising a cage arrangement 2012 having a cone like shape. Cannula 1908 is an outer cannula 1908 of cannula system 1900. Cannula 2010 is an inner cannula 2010 of cannula system 2000. The length and/or outer diameters of cannulas 1908 and 1910 may be different, i.e. inner cannula 1910 may be longer and thinner than outer cannula 1908. Disregarding these differences, both cannulas 1908 and 1910 may correspond to the picture that is shown in FIG. 20. One of the cannulas 2008, 2010 or both cannulas 2008, 2010 may comprise a cage arrangement 2012.

Cannula system 2000 may be adapted or is adapted for a stepwise insertion of cannulas 2008 and 2010, i.e. first outer cannula 2008 and then inner cannula 2010. Dual lumen cannula system 2000 may comprise, preferably as separates systems for each cannula 2008 and 2010 or only one system for both cannulas 2008 and 2010:

a locking mechanism 2002 that may lock an introducer or introducer member that is used for introducing outer cannula 2008 or inner cannula 2010, especially after outer cannula 2008 has been introduced. FIG. 21 shows an example in which an introducer is inserted into a cannula system 2000. The introducer may be locked by force fitting or by another appropriate mechanism.

an adapter portion 2004 that may be used to connect locking mechanism 2002 removably to cannula system 2000, and

a handle portion 2006 that may also be connected removably to cannula system 2000 and that is used to ease introducing of outer cannula 2008 or inner cannula 2010 into body 100 of a patient.

Inner cannula 2008 and/or outer cannula 2010 may comprise a cage arrangement 2012 having wires that extend in the proximal portion of cage arrangement 2012 essentially radially outward. Within an optional short transition portion of cage arrangement 2012 the wires are parallel to each other and/or to the longitudinal axis of cannula 2008, 2010. Within a distal portion of cage arrangement 2012 the wires are arranged on a surface that would define the inclined surface of a cone. This distal portion may extend along almost the entire axial length of cage arrangement 2012 or along at least 90 percent of this length. Thus, it may be said that cage arrangement 2012 has the shape of a cone. Within the distal portion of cage arrangement 2012 the distances between neighboring wires are decreasing with increasing distance to a mounting portion of cage arrangement 2012.

Cage arrangement 2012 of inner cannula 2008 and/or outer cannula 2010 may have or may comprise a membrane, for instance a membrane that has an opening facing distally or laterally, see for instance FIGS. 7 and 8 and corresponding description.

Cannula 2008 and/or cannula 2010 may be pre-bended as described above for cannula system 2000. The tip of cannula 2008 and/or 2009 may be optional, i.e. there may be only one opening within cage arrangement 2012, for instance at its proximal end.

The cage arrangement 2012 may be used also for a single lumen cannula, preferably with or without a membrane.

FIG. 21 illustrates cannula system 2000 in a state in which an introducer 2114 stretches the cage arrangement 2012 for introducing cannula 2008 or 2010 into body 100. Introducer 2114 may also be named as mandrel. Introducer 2114 has a proximal end 2114p and a distal end 2114d. Proximal end 2114p may be clamped within locking mechanism 2002 in the position in which cage arrangement 2012 is stretched enabling the practitioner or the physician to fully concentrate on careful introduction of cannula system 2000 into body 100. Distal end 2114d may be adapted to engage with cage arrangement 2012, preferable with the distal tip and/or the distal portion of cage arrangement 2012. Distal end 2114d may be tapered. If cannula 2008, 2010 is in place, introducer 2114 is unlocked by operating locking mechanism 2002. Thereafter, introducer 2114 is pulled out of cannula 2008, 2010.

At the end of the medical treatment, introducer 2114 may be used again to stretch cage arrangement 2012 and to remove cannula 2008, 2010 out of body 100.

The diameter of introducer 2114 is adapted to have only small slit/gap between an outer surface of introducer 2114 and an inner surface of cannula 2008, 2010. The slit/gap may be smaller than 0.5 millimeter or smaller than 250 micron (micrometer). However, the slit/gap may be greater than 100 micron to allow axial movement of introducer 2114 within cannula 2008, 2010.

Alternatively, introducer 2114 may also be used for cannula system 1900 or for other cannula systems, for instance the cannula systems that are shown in FIGS. 1 to 14. Introducer 2144 may also be used for cage arrangements (diameter variable arrangements) that comprise a membrane, see for instance FIGS. 16 and 17. An adapted introducer may be used to introduce cannulas comprising cage arrangement 1800 that is shown in FIG. 18.

FIG. 22 illustrates an alternative embodiment of a circuitry 2206 wherein a cannula 2240a is pierced or punctured through a ventricle septum VS of heart H. Circuitry 2206 is extracorporeal. Circuitry 2206 may comprise or may consist of:

single lumen cannula 2240a, inserted preferably endovascular jugular,

a single lumen cannula 2240b, inserted preferably endovascular jugular,

a pump P22, and

an oxygenator OXY22.

A proximal end of cannula 2240b may be connected to an input port of oxygenator OXY22 via a flexible tube 2220. An output port of oxygenator OXY22 may be connected to an input port of pump P22, for instance via a tube 2240. An outlet port of pump P22 may be connected to a proximal end of cannula 2240a.

Cannula 2240a may carry a cage arrangement 2246 at its distal end. Cage arrangement 2246 may be placed within ascending aorta aAO. Cage arrangement 2246 may comprise a membrane, for instance a membrane having an opening that faces distally. Alternatively, cage arrangement 2246 may not have a membrane. Cannula 2240a is inserted endovascular through left jugular vein, superior vena cava SVC, right atrium RA, right ventricle RV, ventricle septum VS, left ventricle LV up to ascending aorta aAO. Especially cage arrangement 2246 may be placed within ascending aorta aAO. Only a smart part of the distal end of cannula 2240a may be located within cage arrangement 2246 and therefore also within ascending aorta aAO, for instance less than 5 mm. Thus, cage arrangement 2246 does not comprise a separate distal tip (for instance made of a different material compared to the material of cannula 2240a) that has lateral side holes and/or a distal end-hole. However, in an alternative embodiment, cage arrangement 2246 may comprise a distal tip that has lateral side holes and/or a distal end-hole.

Cannula 2240b may carry a cage arrangement 2286 at its distal end. Cage arrangement 2286 may be placed within left atrium LA preferably transseptal through the atrial septum of the heart H. Cage arrangement 2286 may comprise a membrane, for instance a membrane having an opening that faces laterally. Alternatively, cage arrangement 2286 may not have a membrane. Cannula 2240b is inserted endovascular through right jugular vein, superior vena cava SVC, atrial septum AS into left atrium LA. Especially cage arrangement 2286 may be placed within left atrium LA. Only a smart part of the distal end of cannula 2240b may be located within cage arrangement 2286 and therefore also within left atrium LA, for instance less than 5 mm. Thus, cage arrangement 2286 does not comprise a separate distal tip (for instance made of a different material compared to the material of cannula 2240b) that has lateral side holes and/or a distal end-hole. However, in an alternative embodiment, cage arrangement 2286 may comprise a distal tip that has lateral side holes and/or a distal end-hole.

Alternatively, it is possible to insert cannula 2240a through right internal jugular vein rIJV and cannula 2240b through left internal jugular vein lIJV. Guide wires and/or introducer members may be used in all cases for the insertion of cannula 2240a and 2240b.

Pump P22 drives a drainage flow that comes in through all four pulmonary (see arrows 2297) veins PV out of left atrium LA, through cannula 2240b, tube 2220, oxygenator OXY22, pump P22, tube 2240 and finally through cannula 2240a into ascending aorta aAO, see arrow 2270. Pump P22 may be operated in pulsed mode or may be a pump that generates a pulsatile blood flow, for instance a roller pump. Synchronization to the diastole and systole phases of heart pumping is possible if a sensor is used, for instance a blood pressure sensor. Alternatively, blood pump P22 may generate a continuous blood flow.

Alternatively, cannula 2240a may be a dual lumen cannula or a multi lumen cannula. Cannula 2240b may be omitted if cannula 2240a is a dual lumen cannula or a single lumen cannula. If cannula 2240a is omitted and if cannula 2240 is a dual lumen cannula the outer cannula may be used to drain blood from left ventricle LV.

The ventricle septum VS may be a preferred place for puncturing, for instance if the atrial septum may not be used. Other medical devices may be placed within atrial septum or it may have been punctured too often. There may also be a disease of the atrial septum. However, even without special reasons the ventricle septum VS may be used and not the atrial septum.

The ventricle septum VS may be used for instance in variants of the following embodiments:

in the embodiment that is shown in FIG. 1 for cannula 110,

in the embodiment that is shown in FIG. 2 for cannula 210, a separate cannula may be used with inlet portion 250 in left atrium LA,

in the embodiment that is shown in FIG. 4 for cannula 410,

in the embodiment that is shown in FIG. 5 for cannula 510,

in the embodiment that is shown in FIG. 6 for cannula 640,

in the embodiment that is shown in FIG. 8 for cannula 840,

in the embodiment that is shown in FIG. 9 for cannula 910,

in the embodiment that is shown in FIG. 10 for cannula 1040,

in the antegrade variant of the embodiment that is shown in FIG. 10 for cannula 1040,

in the antegrade lobe dedicated variant of the embodiment that is shown in FIG. 10 for cannula 1040.

It is possible to avoid two cannulas within the right ventricle RV if the main pulmonary artery PA or especially the right pulmonary artery rPA or the left pulmonary artery lPA are reached transcaval from vena cava VC, especially from superior vena cava SVC by puncturing of the vena cava VC and of the respective pulmonary artery PA, see for instance FIG. 25. The cannula that takes the transcaval “short cut” may be inserted endovascular jugular, preferably through one of the interior jugular veins, e.g. left internal jugular vein lIJV or right internal jugular vein rIJV. However, endovascular femoral access to vena cava VC and then transcaval to one of the pulmonary arteries PA is possible as well. The transcaval cannula may be a single lumen cannula.

FIG. 23 illustrates a further alternative embodiment of a circuitry 2306 wherein a dual lumen cannula 2310 is pierced or punctured through ventricle septum VS. Circuitry 2306 is extracorporeal. Circuitry 2306 may comprise or may consist of:

multi lumen or dual lumen cannula 2310, inserted preferably endovascular jugular,

a pump P23, and

an oxygenator OXY23.

A proximal end of the outer cannula of dual lumen cannula 2310 may be connected to an input port of pump P23 via a flexible tube 2320. An output port of pump P23 may be connected to an input port of oxygenator OXY23, for instance via a tube 2340. An outlet port of oxygenator OXY23 may be connected to a proximal end of the inner cannula of dual lumen cannula 2310.

The inner cannula of dual lumen cannula 2310 may carry a cage arrangement 2346 at its distal end. Cage arrangement 2346 may be placed within ascending aorta aAO. Cage arrangement 2346 may comprise a membrane, for instance a membrane having an opening that faces distally. Alternatively, cage arrangement 2346 may not have a membrane.

Cannula 2310 may be a fixed dual lumen cannula or a non-fixed dual lumen cannula. A fixed dual lumen cannula 2310 is inserted endovascular through right jugular vein or left jugular vein, superior vena cava SVC, right atrium RA, right ventricle RV, ventricle septum VS, left ventricle LV up to ascending aorta aAO. Especially cage arrangement 2346 may be placed within ascending aorta aAO. Only a smart part of the distal end of the inner cannula of cannula 2310 may be located within cage arrangement 2346 and therefore within ascending aorta aAO, for instance less than 5 mm. Thus, cage arrangement 2346 does not comprise a separate distal tip (for instance made of a different material compared to the material of the inner cannula of dual lumen cannula 2310 that has lateral side holes and/or a distal end-hole. However, in an alternative embodiment, cage arrangement 2346 may comprise a distal tip that has lateral side holes and/or a distal end-hole.

The outer cannula of dual lumen cannula 2310 may have an inlet portion 2390 comprising a group of inlet holes, for instance a number of holes within the range from 4 to 20. Inlet portion 2390 may be placed within the right atrium RA if cannula 2310 is in its final position within heart H. Additionally or alternatively, the outer cannula of dual lumen cannula 2310 may have an inlet portion 2398 comprising a group of inlet holes, for instance a number of holes within the range from 4 to 20. Inlet portion 2398 may be placed within the right ventricle RV if cannula 2310 is in its final position within heart H.

There may be an optional cage arrangement around inlet portion 2390. An outer sheet member may be used to hold this cage arrangement in its closed or non-expanded state during insertion of dual lumen cannula. An optional cage arrangement may be used around inlet portion 2398. An outer sheet member may be used to hold this cage arrangement in its closed or non-expanded state during insertion of dual lumen cannula. If a fixed dual lumen cannula is used. For a non-fixed dual lumen cannula it is possible to use an introducer member to bring the cage arrangement around inlet holes 2398 in its non-expanded state.

Alternatively, if cannula 2310 is a non-fixed dual lumen cannula it is possible to insert outer cannula first, i.e. through left internal jugular vein HIV or right internal jugular vein rIJV, through vena cava VC, right atrium RA up to left ventricle LV. After the insertion of outer cannula of dual lumen cannula 2310 inner cannula is inserted through outer cannula and then through ventricle septum VS, left ventricle and up to ascending aorta aAO.

Guide wires and/or introducer members may be used in all cases for the insertion of cannula 2310 in one step or in two single steps that are performed in sequence, i.e. first insertion of outer cannula and then insertion of inner cannula.

Pump P23 drives a drainage flow from right atrium (see arrow 2392) and/or from right ventricle RV (see arrow 2399) through outer cannula of dual lumen cannula 2310, tube 2320, pump P23, oxygenator OXY22, tube 2330 and finally through inner cannula of dual lumen cannula 2310 into ascending aorta aAO, see arrow 2370. Pump P23 may be operated in pulsed mode or may be a pump that generates a pulsatile blood flow, for instance a roller pump. Synchronization to the diastole and systole phases of heart pumping is possible if a sensor is used, for instance a blood pressure sensor. Alternatively, blood pump P23 may generate a continuous blood flow.

The ventricle septum VS may be a preferred place for puncturing, for instance if the atrial septum may not be used. Other medical devices may be placed within atrial septum or it may have been punctured too often.

The ventricle septum VS may be used for instance in instead of the embodiment that is shown in FIG. 3.

FIG. 24 illustrates an alternative embodiment of a circuitry 2406 wherein a cannula L15b may be punctured transcaval from vena cava VC to the aorta AO. Circuitry 2406 may comprise or consist of:

a single lumen cannula L15a,

single lumen cannula L15b,

a pump P15, and

an optional oxygenator OXY.

A proximal end of cannula L15a may be connected to an inlet port of pump P15, for instance via a flexible tube. An outlet port of pump P15 may be connected to the proximal end of cannula L15b, for instance via a flexible tube. An oxygenator OXY and/or a carbon dioxide removal unit and/or an adsorber/filter unit and/or another medical device may be included within circuitry 2406 at an appropriate location.

Cannula L15a may be a single lumen cannula that carries at least one expandable arrangement, for instance a cage arrangement, especially a cage arrangement as describes above. If a cage arrangement is used, a membrane may be used as well that is connected to the cage arrangement. However, at least one cage arrangement without a membrane may be used.

Cannula L15a may be inserted endovascular through left internal jugular vein lIJV or through right internal jugular vein rIJV or through another appropriate vessel. Cannula L15a is farther inserted through vena cava VC into the right atrium RA and/or into the right ventricle RV. An inlet portion comprising a group of inlet holes may be arranged within right atrium RA on cannula L15a. Alternatively or additionally, an inlet portion comprising a group of inlet holes may be arranged within right ventricle RV on cannula 15a.

Cannula L15b may be a single lumen cannula that carries an expandable arrangement, for instance a cage arrangement, especially a cage arrangement as describes above, or a balloon, see description of FIGS. 17 and 18 below. If a cage arrangement is used, a membrane may be used as well that is connected to the cage arrangement. The membrane may have an opening that faces distally with regard to the longitudinal axis of cannula L15b.

Cannula L15b may be inserted endovascular through left internal jugular vein lIJV or through right internal jugular vein rIJV or through another appropriate vessel. Cannula L15b is farther inserted through vena cava VC, especially through superior vena cava SVC, through a hole within the wall of vena cava VC, especially a hole in superior vena cava SVC, transcaval to a hole within ascending aorta aAO up to the ascending aorta aAO, where it is fixed for instance by the expandable arrangement mentioned above.

Pump P15 may drive a drainage flow from right atrium RA (see arrow) and/or from right ventricle RV through cannula L15a, pump P15 and finally through cannula L15b into ascending aorta aAO, see arrow. Pump P15 may be operated in pulsed mode or may be a pump that generates a pulsatile blood flow, for instance a roller pump. Synchronization to the diastole and systole phases of heart pumping is possible if a sensor is used, for instance a blood pressure sensor. Alternatively, blood pump P15 may generate a continuous blood flow.

Optional oxygenator OXY may increase the oxygen content of the blood extracorporeal. Thereby, carbon dioxide may be removed.

Alternatively a split tip cannula may be used that comprises both cannulas L15a and L15b.

The atrial septum AS and/or the ventricle septum VS may not be a preferred place for puncturing, for instance if other medical devices are placed within the atrial septum and/or ventricle septum or if one of these septa has or both have been punctured too often. There may also be a disease affecting one or both of the atrial septum and/or of the ventricle septum. Furthermore, the proposed transcaval shortcut from vena cava VC, preferably from superior vena cava SVC, to ascending aorta aAO may be used if the valves of heart H do not function properly any more, for instance because of a disease. However, even without special reasons the shortcut to the aorta may be chosen and not a way through one of the septa.

The following embodiments may be modified:

FIG. 2, cannula L15b may be used for inner cannula of dual lumen cannula 210, outer cannula of dual lumen cannula 210 may still be used as a single lumen cannula,

FIG. 3, cannula L15b may be used for inner cannula of dual lumen cannula 310, outer cannula of dual lumen cannula 210 may still be used as a single lumen cannula,

FIG. 10, cannula L15b may be used for inner cannula of dual lumen cannula 1040, outer cannula of dual lumen cannula 1040 may still be used as a single lumen cannula,

FIG. 10, antegrade variant, cannula L15b may be used for inner cannula of dual lumen cannula 1040, outer cannula of dual lumen cannula 1040 may still be used as a single lumen cannula,

FIG. 10, antegrade lobe-dedicated variant, cannula L15b may be used for inner cannula of dual lumen cannula 1040, outer cannula of dual lumen cannula 1040 may still be used as a single lumen cannula.

FIG. 25 illustrates a further alternative embodiment wherein a cannula 16b is punctured transcaval from vena cava VC to main pulmonary artery PA or to right pulmonary artery rPA or to left pulmonary artery lPA. Circuitry 2506 may comprise or consist of:

a single lumen cannula L16a,

single lumen cannula L16b,

a pump P16, and

an optional oxygenator OXY.

A proximal end of cannula L16a is connected to an inlet port of pump P16, for instance via a flexible tube. An outlet port of pump P16 may be connected to the proximal end of cannula L16b, for instance via a flexible tube. An oxygenator OXY and/or a carbon dioxide removal unit and/or an adsorber/filter unit and/or another medical device may be included within circuitry 2506 at an appropriate location.

Cannula L16a may be a single lumen cannula that carries at least one expandable arrangement, for instance a cage arrangement, especially a cage arrangement as describes above. If a cage arrangement is used, a membrane may be used as well that is connected to the cage arrangement. However, at least one cage arrangement without a membrane may be used.

Cannula L16a may be inserted endovascular through left internal jugular vein lIJV or through right internal jugular vein rIJV or through another appropriate vessel. Cannula L16a is farther inserted through vena cava VC into the right atrium RA and/or into the right ventricle RV. An inlet portion comprising a group of inlet holes may be arranged within right atrium RA on cannula L16a.

Alternatively or additionally, an inlet portion comprising a group of inlet holes may be arranged within right ventricle RV on cannula L16a.

Cannula L16a may be a single lumen cannula that carries an expandable arrangement, for instance a cage arrangement, especially a cage arrangement as describes above.

Cannula L16b may be inserted endovascular through left internal jugular vein lIJV or through right internal jugular vein rIJV or through another appropriate vessel. Cannula L16b is farther inserted through vena cava VC, especially through superior vena cava SVC, through a hole within the wall of vena cava VC, especially a hole in superior vena cava SVC, transcaval to a hole within main pulmonary artery PA or to right pulmonary artery rPA or to left pulmonary artery lPA up to main pulmonary artery PA or to right pulmonary artery rPA or to left pulmonary artery lPA, where it is fixed for instance by the expandable arrangement mentioned above.

Cannula L16b may be a single lumen cannula that carries an expandable arrangement, for instance a cage arrangement, especially a cage arrangement as describes above, or a balloon, see description of FIGS. 17 and 18 below. If a cage arrangement is used, a membrane may be used as well that is connected to the cage arrangement. The membrane may have an opening that faces distally with regard to the longitudinal axis of cannula L16b.

Pump P16 may drive a drainage flow from right atrium RA (see arrow) and/or from right ventricle RV through cannula L16a, pump P16 and finally through cannula L16b into main pulmonary artery PA, right pulmonary artery rPA or left pulmonary artery, see arrow. Pump P16 may be operated in pulsed mode or may be a pump that generates a pulsatile blood flow, for instance a roller pump. Synchronization to the diastole and systole phases of heart pumping is possible if a sensor is used, for instance a blood pressure sensor. Alternatively, blood pump P16 may generate a continuous blood flow.

Optional oxygenator OXY may increase the oxygen content of the blood extracorporeal. Thereby, carbon dioxide may be removed.

Alternatively a split tip cannula may be used that comprises both cannulas L16a and L16b.

The atrial septum AS and/or the ventricle septum VS may not be a preferred place for puncturing, for instance if other medical devices are placed within the atrial septum AS and/or ventricle septum VS or if one of these septum or has or both have been punctured too often. There may also be a disease affecting one or both of the atrial septum AS and/or of the ventricle septum VS. Furthermore, the proposed transcaval shortcut from vena cava, preferably from superior vena cava SVC, to main pulmonary artery PA, to right pulmonary artery rPA or to left pulmonary artery may be used if the valves of heart H do not function properly any more, for instance because of a disease. However, even without special reasons shortcut to the pulmonary artery (PA, rPA, lPA) may be chosen and not a way through one of the septa AS, VS.

The following embodiments may be modified for instance:

in the embodiment that is shown in FIG. 6, i.e. in a pECLA (percutaneous extra corporeal lung assist) circuitry 606, cannula L16b may be used for a single lumen cannula 610 that extends from vena cava VC through right atrium RA and right ventricle into main pulmonary artery PA, in the embodiment that is shown in FIG. 9 cannula L16b may be used for cannula 910, cannula L16a is not necessary,

in the embodiment that is shown in FIG. 10 cannula L16b may be used for inner cannula of dual cannula 1010, the inlet portion(s) 1090 and/or 1098 of outer cannula of cannula 1010 may still be used, cannula L16a is not necessary,

in the antegrade variant of the embodiment that is shown in FIG. 10 cannula L16b may be used for inner cannula of dual cannula 1010, the inlet portions 1090 and/or 1098 of outer cannula of cannula 1010 may still be used, cannula L16a is not necessary, and

in the antegrade lobe-dedicated variant of the embodiment that is shown in FIG. 10 cannula L16b may be used for inner cannula of dual for cannula 1010, the inlet portions 1090 and/or 1098 of outer cannula of cannula 1010 may still be used, cannula L16a is not necessary.

FIG. 26 illustrates a cannula L17 that carries an inflatable expandable arrangement Ba, for instance a balloon. Balloon Ba may have a cylindrical shape and may be connected to a distal portion of cannula L17, for instance using an adhesive.

A channel CH1 may be arranged on an outer surface of cannula L17. Channel CH1 may extend from a proximal part of cannula L17 up to balloon Ba. If a fluid is driven into channel CH1 balloon Ba inflates. If the fluid is driven out of channel CH1 then balloon Ba deflates.

Thus, balloon Ba may form a border element that is between cannula L17 and a vessel V of the blood circuit. Vessel V may be a pulmonary artery of lung L or a pulmonary vein of lung L. There may be a transport volume TrV that is used to treat lung L and that is on the distal side of inflated balloon Ba. The natural blood circuit BC may be on the proximal side of balloon Ba. Balloon Ba may isolate the natural blood circuit BC from transport volume TrV.

Transport volume TrV is directly in fluidic connection with cannula L17 through the holes in a separate distal tip Ti17, see for instance holes Ho17. Alternatively, cannula L17 may have only a single end-hole EH at its distal end.

Cannula L17 may be one of the cannulas mentioned above during the description of FIGS. 1 to 11 and 15 to 23.

Alternatively and/or additionally, channel CH1 may be arranged within cannula L17. Cannula L17 may be a single lumen cannula or a multi lumen cannula, especially the inner cannula or the outer cannula of a dual lumen cannula. A combination of an internal channel and an external channel is possible as well.

Deflated balloon Ba may not have a further protection shield during insertion of cannula L17. However, alternatively a removable sheath may be wrapped around balloon Ba during insertion of cannula L17.

FIG. 27 illustrates a split tip cannula L18 that carries two expandable arrangements on its two distal end lumens L18a and L18b. An inner lumen of cannula L18 bifurcates in distal end lumen L18a and distal end lumen L18b at a bifurcation point or bifurcation position Bi. Each of the expandable arrangements may be a balloon Ba as shown in FIG. 17 and described above. A channel CH2 may correspond to channel CH1 mentioned above. Channel CH2 may be connected to a channel CH2a that extends on distal end lumen L18a to the respective balloon and to a channel CH2b that extends on distal end lumen L18b to the respective balloon. Alternatively or additionally internal channels may be used to inflate or deflate the balloons. A combination of an internal channel and an external channel is possible as well. Furthermore, it is possible to use two separate channels CH2a1 and CH2b1 that extend from a proximal part of cannula L18 to either distal end lumen L18a or distal end lumen L18b. Separate and independent control of balloon on distal end lumen L18a and on distal end lumen L18b is possible in this variant. Introduction and fixation of cannula L18 is easier with separate control of both balloons.

Alternatively, two cage arrangements may be used on the distal end lumens L18a and L18b. Cage arrangements that are mentioned above may be used, see for instance FIGS. 1 to 11, 15 to 23. An introducer member 118 may be used that has a split tip, i.e. a bifurcation. Alternatively two separate introducer members may be used within cannula L18, one extending into distal end lumen L18a and the other extending into distal end lumen L18b.

Cannula L18 may be a single lumen cannula having a split tip. Alternatively, cannula L18 may comprise two separate lumens, one connected to lumen L18a and the other connected to lumen L18b.

Furthermore, for each embodiment of cannula L18 described above cannula L18 may not be inserted into a further cannula or may be inserted in a further dual lumen cannula, for instance in a fixed dual lumen cannula or multi lumen cannula or non-fixed cannula dual lumen cannula or multi lumen cannula.

The treatment fluid flow may be heated in order to improve the uptake of medicaments/treatment substances by the tissue of the organ and/or by the cells of the organ. If the treatment fluid comprises blood or a high percentage of blood or blood components the heating temperature may be for instance in the range between 39.0 and 44.0° C. (degrees of Celsius), preferably to between 40.0 and 42.5° C.

However, due to the isolation of the transport volume from the body fluid and/or due to the local treatment even higher temperatures may be used, especially if the fluid flow through the transport volume does not contain or comprise blood or blood components or only a lower percentage of blood per volume. Therefore, also temperatures above 42.5° C. may be used, for instance above normal blood temperature, above 43° C., above 44° C., above 45° C. or even above 50° C. This may improve the uptake of medicaments/treatment substances further.

The term “normal body temperature (also known as normothermia or euthermia)”, as used herein, may refer to the typical temperature found in an individual. In humans, the normal body temperature is 37° C. This value is, however, only an average. The normal body temperature may be slightly higher or lower. A number of factors can influence the body temperature, including age, sex, time of day, and activity level. In babies and children, for example, the average body temperature ranges from 36.6° C. to 37.2° C. Among adults, the average body temperature ranges from 36.1° C. to 37.2° C. The normal human body temperature range is, thus, typically stated as being between 36.1° C. and 37.5° C., e.g. 36.1, 36.2, 36.3, 36.4, 36.5, 36.6, 36.7, 36.8, 36.9, 37.0 37.1, 37.2, 37.3, 37.4, or 37.5° C., in humans.

Furthermore, it is possible to use in all embodiments that are mentioned above an inner surface of the lumen portion and/or inner lumen that comprises a spirally and/or helically surface structure. The spirally and/or helically surface structure may have the effect that the fluid flow within the cannula is rotated as it moves through the cannula. Turbulences may be reduced thereby and/or it may be possible to reach much higher flow rates compared to cannulas that have a smooth inner surface, i.e. that do not have spirally and/or helical surface structures on their inner surfaces. However, it is of course possible to use cannulas without a spirally and/or helical surface features, if for instance lower flow rates are necessary. The spirally turned flow and/or the rotated flow may prevent clotting of blood cells if the fluid flow comprises blood, especially in slow flow rate conditions. However, there may also be advantages if the fluid flow does not contain blood. The spiral flow may be a laminar spiral flow.

There may be an embodiment in which a single lumen cannula or a dual or multi lumen cannula is used (fixed or non-fixed) wherein the single lumen cannula or the inner cannula of the dual or multi lumen cannula may have a split tip. Each distal tip of the split tip cannula may be associated with or may be carry an expandable arrangement, for instance a balloon or a cage, especially with a cage that carries a membrane. The distal parts of the split tip cannula may be inserted into the left pulmonary veins whereby the right pulmonary veins may be left open. Alternatively, the distal parts of the split tip cannula may be inserted into the right pulmonary veins whereby the left pulmonary veins may be left open.

Furthermore, there may be an embodiment in which a single lumen cannula or a dual cannula is used (fixed or non-fixed) wherein the single lumen cannula or the inner cannula of the dual lumen or multi lumen cannula may have a cage arrangement on its distal end. The cage arrangement may carry a membrane. The membrane may define an opening that faces laterally. If the cannula is inserted into the body, the opening of the membrane may face laterally in the direction of both right pulmonary veins. Both left pulmonary veins may remain open, i.e. blood may pass the outside of the membrane thus not entering the cannula that carries the cage arrangement with the membrane.

The following feature combinations may be relevant:

a) All embodiments that relate to a cage arrangement or to another expandable arrangement may be used to reach a stable and/or secure positioning or fixation of the cannula in the chambers of the heart (left atrium LA, right atrium RA, left ventricle LV, right ventricle RV) or vessels of the blood circuit. The cage arrangement or to another expandable arrangement may allow a better design of the cannula, especially of the distal tip, for instance only one end-hole may be used instead of multi-hole distal parts. This may result in better or optimal flow characteristics, for instance less shear stress and/or less turbulences and/or less shear rates and/or less recirculation and/or more homogeneous velocity profile, etc.

The cannula may comprise at least one end hole or a single end-hole through which at least 25 volume percent, at least 50 volume percent, at least 75 volume percent or at least 90 volume percent or all of the flow flows into or out of the cannula, all volumes measured for instance for 3.5 l per minute or for 5 l per minute. The given volume percent of flow through the at least one end-hole may be measured for instance for a flow through the cannula of 3.5 l per minute or for 5 l per minute. Thus, a significant portion of the flow flows through the at least one end hole. Additionally, the cannula may be a “tip-less” cannula which extends not or only at most 3 mm (millimeter) within the inner volume of the cage in the expanded state. Thus, the fixation of the cage may extend only up to the distal end of the cannula or up to a location which is equal to 3 mm or at most 3 mm away from the distal end of the cannula.

In another embodiment at least one side hole may be present in the cannula in which at least 25 volume percent, at least 50 volume percent, at least 75 volume percent or at least 90 volume percent or all of the flow flows into or out of at least one end-hole of the cannula and/or in the case of a tip-less cannula having an axial oriented inlet or outlet. The at least one side hole may have at least one other function in addition to the function of flow transport into or out of the cannula. The flow transport through the at least one side-hole may be equal to or less than 50 volume percent of the overall flow through the cannula or equal to or less than 25 volume percent of the overall flow through the cannula. The given volume percent of flow through the at least one side hole may be measured for instance for a flow through the cannula of 3.5 l per minute or for 5 l per minute.

b) All embodiments may be used with fix or non-fixed dual lumen cannulas or multi lumen cannulas. If non-fixed (may be inserted into each other) cannulas are used, it is easier to position or implant the cannulas stepwise along a curved introduction path because less friction may be involved, especially at the puncture site, and the insertion may be less traumatic.

c) All embodiments may be used for endovascular access and for in-vivo lung isolation which may allow an isolated perfusion of lung L or of parts of lung L, for instance in a closed circuit of fluid flow that may be isolated from the body blood circuit.

d) If within the cage a cannula tip is used which has side-holes it is possible to have at least one end-hole or to have no end-holes.

The combination of at least two arbitrarily selected or of all feature combinations a), b), c) and d) may give the best result.

Moreover, the cannula, for instance the delivery cannula, may be inserted endovascular jugular and may be punctured from superior vena cava SVC or from right atrium RA transcaval to ascending aorta aAO. Alternatively, the cannula, for instance the delivery cannula, may be inserted endovascular femoral through inferior vena cava IVC into the right atrium RA and may be punctured from superior vena cava SVC or from right atrium RA transcaval to ascending aorta aAO.

Moreover, the delivery cannula or the drainage cannula may be inserted endovascular jugular and may be punctured from superior vena cava SVC or from right atrium RA transcaval to the main pulmonary artery PA or to the right pulmonary artery rPA or in special cases to the left pulmonary artery lPA. Alternatively, the delivery cannula or the drainage cannula may be inserted endovascular femoral through inferior vena cava IVC into the right atrium RA and may be punctured from superior vena cava SVC or from right atrium RA transcaval to the main pulmonary artery PA or to the right pulmonary artery rPA or in special cases to the left pulmonary artery lPA.

In all embodiments with a cage arrangement it is also possible to use another material than a metal, for instance a natural and/or biological material, especially cellulose, for instance cellulose that is treated to increase the hardness. Compatibility with body 100 and/or with blood may be improved thereby.

FIG. 28 illustrates a blood circulating system 2800 comprising a dual lumen cannula CA28 and a membrane pump MP. In the example, a pRVAD (percutaneous right ventricle assisted device) is illustrated.

The dual lumen cannula CA28 comprises:

an outer cannula O28, and

an inner cannula I28.

Outer cannula O28 may comprise a group of side holes SH on its distal end. If outer cannula O28 extends up to the right ventricle RV there may be a group of side holes SH within the right atrium RA and a group of side holes SH within the right ventricle RV of heart H. Each group of side holes SH may comprise at least one side hole preferably a number of side holes SH within the range of 2 or 3 side holes to 20 side holes SH.

The inner cannula 128 may comprise only one end-hole or at least one end-hole but no side holes. In an alternative embodiment the inner cannula 128 may comprise side holes SH and/or no end-hole EH or at least one additional end-hole EH.

The outer cannula O28 may be inserted first into a body of a patient. Thereafter, the inner cannula I28 may be inserted within the outer cannula O28 and then further forward into the body of the patient.

In the embodiment that is illustrated in FIG. 28 the outer cannula O28 may be inserted up to the right atrium RA or up to the right ventricle RV, for instance via vena cava VC, especially via superior vena cava SVC or via inferior vena cava IVC.

The inner cannula I28 may be inserted into the outer cannula O28 up to the right atrium RA or up to the right ventricle RV depending on where the end of the outer cannula O28 is located and then further into the main pulmonary artery PA, into the right pulmonary artery rPA or into the left pulmonary artery lPA.

Cannula CA28 may have a coupling port P28a which is fluidly connected with outer cannula O28 and a coupling port P28b which is fluidly connected with. The coupling ports P28a and P28b are adapted to be coupled to a blood pump, preferably to the membrane pump MP. Optional flexible conduits may be used to connect ports P28a and P28b with the membrane pump MP which is located outside of the body of a patient in the embodiment.

Membrane pump MP may comprise:

an inlet port PI28, e.g. a blood input port,

an outlet port PO28, e.g. a blood output port,

an auxiliary port PM28 for an auxiliary fluid, especially a gaseous fluid, e.g. helium or air,

a flexible membrane M, for instance comprising polycarbonate, poly(methyl methacrylate) PMMA, silicone, or of another appropriate material, and

a case which holds the parts of the membrane pump MP.

There may be one way valves V28a and V28b within inlet port PI28 and within outlet port PO28. Alternatively, it is possible to arrange the valves V28a and V28b within the connection between ports P28a and PI29 and within the connection between ports P28b and port PO28 or at other appropriate locations. The valves V28a, V28b may be single disc valves or bi-leaflet valves or other appropriate valves. The bi-leaflet design may consists of or may comprise two semicircular leaflets which pivot on hinges. Bi-leaflet valves may have the best central flow—e.g. the leaflets open completely, allowing very little resistance to blood flow.

Auxiliary port PM29 may be connected to an IAB (intra-aortic balloon) console which may already be present in many hospitals. The IAB console may include control unit which follows an ECG (Electrocardiography) signal of the patient. Thus, blood is pumped pulsatile which is the natural way of blood pumping. Pulsatile pumping may have advantageous if compared with continuous pumping, especially with regard to less clotting of the blood. Alternatively, another appropriate device may be used instead of IAB console at auxiliary port PM29. However, pulsatile operation mode may be used also for other driving devices than the IAB console.

This means that at least one variable volume reservoir VVR that has an aspiration phase or an aspiration operating phase for drawing fluid into the variable volume reservoir (but out of the port P28a of outer cannula O28) and that has an expulsion phase or an expulsion operating phase for pressing the fluid out of the variable volume reservoir VVR (e.g. delivery blood into port P28b of inner cannula I28).

Thus, the following blood flow may be provided:

blood may be sucked into side holes SH from right atrium RA and/or right ventricle RV, see arrow 28a,

the blood is then transported through outer cannula O28 to inlet port PI28, see arrow 28a,

the membrane pump MP pumps the blood through outlet port PO28 into inner cannula I28, see arrow 28a, and

the blood flows out of end-hole EH of inner cannula I28 into a pulmonary artery PA, lPA or rPA, see arrow 28a.

In an alternative embodiment an oxygenator device and/or other blood treatment device may be included, e.g. a blood filter device, a hemolysis device and/or a carbon dioxide removal device. The additional device or the additional devices may be included between ports P28a and PI28 and/or between ports PO28 and P28b.

Advantages and technical effects:

pulsatile blood flow,

very gentle pumping for red blood cells and other particles within the blood, e.g. hardly damaged and therefore no or only less thrombosis/blood clotting,

higher pumping volume (liter per minute) may be possible compared to other pumping solutions, e.g. more than 3 liter per minute, more than 3.5 liter per minute or more than 4 liter per minute,

the technical solution is comparably simple.

Contrary, centrifugal pumps may have 5000 to 10000 and more revolutions per minute. These high rotation rates may damage the blood cells, especially the red blood cells.

There may be an expandable arrangement on the distal tips of the outer cannula O28 and/or on the distal tip of the inner cannula I28 in order to have a fixation and/or to prevent damage of neighboring tissue and/or for other purposes. In both cases, the expandable arrangement may be a cage comprising wires, see embodiments mentioned above, an inflatable balloon or another appropriate device. A cage without a membrane may be used. Alternatively, a cage with a membrane may be used as mentioned above.

A dual chamber membrane pump with 40 ml (milliliter) or more and up to 160 ml pumping volume may be used. For all embodiments, a membrane pump, for instance MP with 60 ml or more up to 160 ml pumping volume may be used, most preferred in the range between 80 ml to 120 ml.

At least one pump for driving a fluid flow may be used, for instance a membrane pump (pulsatile flow), especially comprising a flat membrane or a ring membrane or a balloon membrane. The pump volume of the pump may be preferably greater than the volume in the cannula between the distal end of the cannula and the inlet of the pump, especially a membrane pump, i.e. there may be no or only a small dead volume. This may result in no or only less clotting of blood within the cannula and/or the pump or variable volume reservoir.

A membrane pump MP may be uses for all embodiments mentioned above which use a dual lumen cannula having an inner cannula which is insertable into the outer cannula after the outer cannula has been inserted into the body of a patient.

The embodiment of FIG. 28 may be combined with all other embodiments mentioned above, especially with the embodiments of FIGS. 12 to 13.

FIG. 29 illustrates a heart assist system 2900 comprising a dual cannula and a cannula within the coronary sinus vein CSV. FIG. 29 shows an exemplary embodiment of a cannula O1, also referred to as first cannula O1.

The cannula O1 may have a kink K in a base state, i.e. no external forces are applied to the cannula O1. The cannula O1 or its cannula body is bendable or flexible. The cannula O1 comprises a cannula body, which is formed as a tube. A sidewall of the cannula body may comprise a plastic or silicone. The sidewall may comprise an internal metal scaffold or framework. The length of the cannula O1 or of the cannula body O1B may be between 20 cm and 80 cm, i.e. within the range from 20 cm to 80 cm.

The cannula body comprises, in the base state of the cannula O1, a first section S1, a second section S2 and a pre-bended intermediate section K connecting the first section S1 and the second section S2. A bending angle of the intermediate section K is, e.g., 90° (degrees). The first section S1 extends from a distal end of the cannula body to the intermediate section K and adjoins the intermediate section K. The second section S2 adjoins the intermediate section K and extends from the intermediate section K in direction to the proximal end of the cannula O1.

A length of the first section S1 is, for example, between 1 cm and 2 cm, inclusive. The intermediate section K is shorter than the first section S1. The second section S2 has, for example, a length between 5 cm and 10 cm, inclusive. Between the second section S2 and the proximal end, a third section S3 of the cannula O1 is arranged.

In the base state of the cannula O1, the second section S2 has a curvature. A curvature radius of the second section S2 is, for example, between 5 cm and 50 cm, inclusive. The curvature radius of the second section S2 is, preferably, constant over a large part of the length of the second section S2 or varies by at most 10% around an average curvature radius.

Moreover, in the base state, the first section S1 is straight. Also the third section S3 may be straight or almost straight. The length of the third section S3 may be, e.g., greater than 10 cm.

The cannula O1 is a single lumen cannula comprising only one lumen L1, along which a fluid, particularly blood, can be guided. An inner diameter of the cannula body, i.e. the diameter of the lumen L1, is, for example between 10 French and 12 French, inclusive. The outer diameter may be between 2 French and 4 French larger than the inner diameter.

In the region of the distal end of the cannula body, the cannula O1 may comprise a diameter variable arrangement DVA (not illustrated) in the form of a cage arrangement with a plurality of wires. The wires may be made of a super elastic material, e.g. stainless steel, and may each have a thickness between 0.25 mm and 0.75 mm, inclusive. The cage arrangement DVA is in an expanded state, in which its diameter is larger than the diameter of the sections S1, S2, S3 of the cannula body. The cage arrangement DVA projects beyond the distal end of the cannula body by, e.g., 1 cm.

The cage arrangement DVA may further comprises a membrane M, which is also in an expanded state when the cage arrangement is in an expanded state. The membrane M covers a proximal portion of the diameter variable arrangement DVA. An edge of the membrane M may define a distal opening that faces distally relative to the first section S1. The membrane M may have a proximal opening facing proximally with respect to the first section S1. The diameter of the proximal opening may be smaller than the one of the distal opening. The proximal opening radially surrounds the first section S1. A volume defined by the membrane M is fluidically coupled to the lumen L1 of the cannula O1 via an opening at the distal end of the first section S1. The diameter of the opening of the first section S1 at the distal end of the first section S1 is, e.g., the same as the diameter of the lumen L1. The membrane M is, e.g., made of PTFE and has, e.g., a thickness between 10 μm and 50 μm, inclusive.

The diameter variable arrangement DVA and/or the membrane M may be optional. The cannula O1 could also be used for the insertion into the left atrium via the coronary sinus vein CSV without these two elements.

An embodiment of a kit may comprise the cannula O1 and a penetration device PD in the form of a catheter. The catheter PD may comprise a needle or a wire at its distal and. The catheter PD may be inserted into a body before the cannula O1 is inserted into the body. The needle or the wire are configured for producing a hole connecting the lumen of the coronary sinus vein of a heart with the left atrium of the heart, for instance mechanically and/or by using other energies, for instance alternating current, radio frequency, etc. for instance in order to generate heat, especially heat of equal to or more than 50 degrees centigrade. For example, the catheter PD has a smaller outer diameter than the cannula body and/or is more bendable or flexible than the cannula O1. The length of the catheter PD may be approximately the length of the cannula O1. The diameter of the needle may be 5 French. The needle PD1 or wire PD1 may be configured to produce a hole with a diameter of about 5 French in the wall of the heart.

FIG. 29 shows a heart assist system 2900 comprising the cannula O1 and a cannula system S23. Cannula system S23 may be a dual cannula system as mentioned above, see for instance FIG. 10, second dual lumen cannula 1040. The cannula system S23 comprises a second cannula O2 and a third cannula O3, both of them being a single lumen cannula. The third cannula O3 is thinner than the second cannula O2 and is inserted into the second cannula O2. By way of example, an outer diameter of the cannula body of the third cannula O3 is smaller by 8 French than the inner diameter of the cannula body of the second cannula O2 (see also FIG. 10).

The second cannula O2 and the third cannula O3 of the cannula system S23 define two lumens L2, L3, wherein a first lumen L2 is defined between an outer surface of the third cannula O3 and an inner surface of the second cannula O2. A second lumen L3 is defined by the lumen of the third cannula O3 (see also FIG. 10).

The proximal ends of the first cannula O1 and of the cannula system S23 are connected to a pump P. Pump P may be a membrane pump, see for instance membrane pump MP of FIG. 28. A y-connector may be used to couple both inputs. Alternatively pump P may be another pump, for instance a centrifugal pump, a roller pump etc.

In FIG. 29, the first cannula O1 and the cannula system S23 are inserted into a heart H of a body, particularly a human body. The heart H is illustrated from its back side in order to illustrate the coronary sinus vein (SVC) clearly. The heart H is viewed from the backside. The heart H comprises the left ventricle LV, the right ventricle RV, the left atrium LA and the right atrium RA. The inferior vena cava IVC and the superior vena cava SVC are connected to the right atrium RA. The right atrium RA is connected to the right ventricle RV via the tricuspid valve TV. The right ventricle RV is connected to a pulmonary artery PA. The left atrium LA is connected to a pulmonary vein PV. The left atrium LA and the left ventricle LV are connected via the mitral valve MV. Moreover, the left ventricle LV is connected to the aorta AO. The left atrium LA and the right atrium RA are separated from each other by the atrial septum AS.

The cannula/first cannula O1 is inserted into the heart H. In particular, the first cannula O1 is guided intravascularly along the superior vena cava SVC, from there it is inserted into the coronary sinus vein CSV, is guided intravascularly along the coronary sinus vein CSV and is inserted from the coronary sinus vein CSV into the left atrium LA of the heart H, where the first cannula O1 ends. Instead of guiding through the superior vena cava SVC, the first cannula O1 may also be guided intravascularly along the inferior vena cava IVC and from there being inserted into the coronary sinus vein CSV.

The first cannula O1 is inserted into the heart H in such a way that the second section S2 of the first cannula O1 lies in and runs intravascularly along the coronary sinus vein CSV. Particularly, the curvature of the second section S2 in the base state is adapted to the curvature of the coronary sinus vein CSV so that the second section S2 of the first cannula O1 does not induce too much force or stress or pressure onto the coronary sinus vein CSV. The length of the second section is, e.g, the length of the coronary sinus vein±20%. The intermediate section K of the first cannula O1 lies in a region directly opposing the left atrium LA, is sharply bended and thus allows the first section S1 to project into the left atrium LA. For example, the first section S1 projects into the left atrium LA by at least 0.2 cm and at most 2 cm.

The cannula system S23 is inserted into the heart H and is guided intravascularly along the superior vena cava SVC into the right atrium RA. From the right atrium RA, the cannula system S23 is guided through a hole in the atrial septum AS into the left atrium LA. The second cannula O2 ends in the left atrium LA, i.e. its distal end is in the left atrium LA. The third cannula O3 runs inside the second cannula O2, i.e. in the lumen of the second cannula O2, along the entire length of the second cannula O2 and projects beyond the distal end of the second cannula P2. From the distal end of the second cannula O2, the third cannula O3 further runs inside the left atrium through the mitral valve MV into the left ventricle LV. The third cannula O3 might end in the left ventricle LV, i.e. has its distal end in the left ventricle LV. In the present case, however, the third cannula O3 is further inserted through the aortic valve AV into the aorta AO end ends in the aorta AO, e.g. in the ascending aorta AO.

The heart assist system 100 of FIG. 29 may be operated as follows: blood is sucked out from the left atrium LA via the first lumen L2 of the cannula system S23, see arrow A29a (see also FIG. 10). The under pressure necessary for this step is provided by the pump P at the distal end of the cannula system S23. For example, between 21 (Liter) blood per minute and 41 blood per minute are sucked out from the left atrium LA via the cannula system S23. The blood is guided along the first lumen L2 of the cannula system S23 to the proximal end of the cannula system S23, see arrow A29b.

As the blood flow through the first lumen L2 of the cannula system S23 is limited by the area of the first lumen L2 and the maximum acceptable suction pressure, the first cannula O1 is used to support the sucking out of blood from the left atrium LA, see arrow A29c. Blood in the left atrium LA is sucked out via the first cannula O1, is guided along the lumen L1 of the first cannula O1 to the proximal end of the first cannula O1, see arrow A29d. For example, about 1 l (Liter) blood per minute is sucked out via the first cannula O1.

The blood guided along the lumen L1 of the first cannula O1 and along the first lumen L2 of the cannula system S23 is, e.g., guided out of the body. It can then be reinserted into the body with help of the pump P, which reinserts the sucked out blood into the second lumen L3 of the cannula system S23, see arrow A29e. The blood is then guided along the second lumen L3 of the cannula system S23 to the distal end of the third cannula O3 and is there inserted into the aorta AO, see arrow A29f. In this way, the function of the heart as a pump can be supported by the pump P outside of the body.

The third cannula O3 is inserted into the second cannula O2 so that the first lumen L2 is defined between the outer surface of the third cannula O3 and the inner surface of the second cannula O2. The lumen of the third cannula O3 defines the second lumen L3 of the cannula system S23.

A variant of FIG. 29 which is not shown refers to an exemplary embodiment of the heart assist system 2900a, wherein the first cannula O1 and the cannula system S23 are inserted into the heart H of a body as shown in FIG. 29. In difference to FIG. 29, the heart assist system 2900a comprises an oxygenator OXY besides the pump P also located outside of the body. Blood, sucked out with help of the pump P, is injected into the oxygenator OXY, is enriched with oxygen or carbon dioxide is removed, and is then inserted into the second lumen L3 of the cannula system S23 through which it is guided into the aorta AO. Additionally or instead of enriching with oxygen or removing carbon dioxide, the blood could also be enriched with a drug outside of the body.

The embodiments and variants of FIG. 29 may be combined with all other embodiments mentioned above, especially with the embodiments of FIGS. 16 to 21. FIGS. 12 to 14 may be relevant for dual lumen cannula S23 (O2, O3). The usage of single end-hole (i.e. without any side-holes) may result in high flow rates and/or less wall shear stress and/or less shear rates and/or less turbulences and/or less recirculation and/or more homogeneous velocity profile of the blood transported within cannula O1 and/or S23, O2 and/or S23, O3.

However, alternatively cannula O1 and/or S23, O2 and/or S23, O3 may have a distal tip having side-holes SH in combination with a cage arrangement and/or membrane or without a cage arrangement.

FIG. 30 illustrates a pLVAD® assist system (arrangement) 700 with blood B transport from left atrium LA to aorta AO, e.g. to ascending aorta aAO or to descending aorta dAO, with a dual lumen cannula system DL-CS700. This arrangement 700 is similar to arrangement 206 which is illustrated in FIG. 2. However, another pump system is used. Again, cage arrangements may be optionally used.

Dual lumen cannula system DL-CS700 comprises:

an outer cannula CA700a, and

an inner cannula CA700b which is arranged inside of outer cannula CA700A, especially after outer cannula CA700a is inserted into the body of a patient.

Cannula CA700a comprises:

a proximal portion PP700a,

a distal portion DP700a, and

a distal opening DO700a within distal portion DP700a.

Distal opening DO700a is arranged within left atrium LA.

Cannula CA700b comprises in a first variant:

a proximal portion PP700b,

a distal portion DP700b1, and

a distal opening DO700b1 within distal portion DP700b1.

Distal opening DO700b1 is arranged within ascending aorta aAO.

Cannula CA700b comprises in a second variant:

a proximal portion PP700b,

a distal portion DP700b2, and

a distal opening DO700b2 within distal portion DP700b2.

Distal opening DO700b2 is arranged within descending aorta dAO.

Both cannulas CA700a, CA700b of dual lumen cannula system DL-CS700 are inserted through the atrial septum. Outer cannula CA700a may be inserted first. Cannula CA700a may be inserted through one of the jugular veins, through superior vena cava SVC, through right atrium RA, atrial septum AS into left atrium LA. Thereafter, inner cannula CA700b may be inserted into outer cannula CA700a to left atrium LA and then further as described above for cannula CA600b, e.g. there may be again two variants (variant 1 and variant 2).

Arrangement 700 comprises a pump arrangement Arr7a comprising only one membrane pump MP7a which is driven by an IABP (intra-aortic balloon pump) console IABP7.

Arrangement 700 comprises further:

a separated portion SP7a which is connected with proximal portion PP700a of cannula CA700a and with a connecting portion CP7a,

a separated portion SP7b which is connected with proximal portion PP700b of cannula CA700b and with connecting portion CP7a,

a separated portion SP7c which is connected with liquid flow port (reservoir port RP) of membrane pump MP7 and with connecting portion CP7a.

There may be the following one-way valves in arrangement 700:

a one-way valve V7a within separated portion SP7a, and

a one-way valve V7b within separated portion SP7b.

The function of arrangement 700 is as follows:

an arrow A700a illustrates inflow in outer cannula CA700a from left atrium LA,

an arrow A700b illustrates inflow through separated portion SP7a and through one-way valve V7a,

an arrow A700c illustrates outflow through separated portion SP7b and through one-way valve V7b into cannula CA700b,

an arrow A700d illustrates outflow through distal opening DO700b1 (variant 1), and

an arrow A700e illustrates outflow through distal opening DO700b2 (variant 2).

Alternatively, a pump arrangement Arr7b may be used instead of pump arrangement Arr7a which comprises only one membrane pump MP7. Pump arrangement Arr7b comprises:

membrane pumps MP7a and MP7b,

a connecting portion CP7b,

a separated portion SP7d which is connected to the liquid flow port of membrane pump MP7a,

a separated portion SP7e which is connected to the liquid flow port of membrane pump MP7b,

a separated portion SP7f which is connected to connecting portion SP7c, and

a connecting portion CP7c, which corresponds to connecting portion CP7b mentioned above, e.g. it is between gas ports of membrane pumps MP7a and MP7b and an IABP console IABP7a.

Connecting portion CP7b is arranged between separated portions SP7d to SP7f. Connecting portion CP7b may be a Y-connector or a T-connector or another 3 port element. Alternatively an X-connector may be used which comprises both connecting portions CP7a and CP7b.

Three way stop cocks 3WSC7a, 3WSC7b may be used in pump arrangement Arr7b in order to ease changing and/or stopping and/or removing of at least one of the membrane pump MP7a, MP7b during operation of arrangement 700 and preferably also of pump arrangement Arr7b.

FIG. 31 illustrates a pBiVAD assist system or arrangement 1100 with blood B transport from left atrium LA and right atrium RA to aorta AO, e.g. to ascending aorta aAO or to descending aorta dAO, using a dual lumen cannula system DL-CS1000 and one single lumen cannula CA1100c which is a separate cannula.

Dual lumen cannula system DL-CS1000 comprises:

an inner cannula CA1100a, and

an outer cannula CA1100b.

Inner cannula CA1100a comprises:

a proximal portion PP1100a,

a distal portion DP1100a, and

a distal opening DO1100a within distal portion DP1100a.

Distal opening DO1100a is arranged within left atrium LA.

Outer cannula CA1100b comprises:

a proximal portion PP1100b,

a distal portion DP1100b, and

a distal opening DO1100b or several distal openings DO1100b within distal portion DP1100b.

Distal opening(s) DO1100b is (are) arranged within right atrium RA.

Outer cannula CA1100b may be inserted through superior vena cava SVC into right atrium RA. Thereafter, inner cannula CA1100a may be inserted into outer cannula CA1100b and further through right atrium RA, through atrial septum AS into left atrium LA.

Cannula CA1100c may be inserted through superior vena cava SVC into right atrium RA and then transseptal through atrial septum AS, through left atrium LA, through left ventricle LV into ascending aorta aAO (variant 1) or up to descending aorta dAO (variant 2).

The function of arrangement 1100 is as follows:

an arrow A1100a illustrates inflow into inner cannula CA1100a from left atrium LA,

an arrow A1100b illustrates outflow from proximal portion PP1100a of inner cannula CA1100a, see FIGS. 31A and 31B,

an arrow A1100c illustrates inflow into distal opening(s) of outer cannula CA1100b from right atrium RA,

an arrow A1100d illustrates outflow from proximal portion PP1100b of outer cannula CA1100b, see FIGS. 31A and 31B,

an arrow A1100d illustrates inflow into proximal portion PP1100c of single lumen cannula CA1100c, see FIGS. 31A and 31B,

an arrow A1100f illustrates outflow through distal opening DO1100b1 (variant 1) of single lumen cannula CA1100c into ascending aorta aAO, and

an arrow A1100g illustrates outflow through distal opening DO1100b2 (variant 2) of single lumen cannula CA1100c into descending aorta dAO.

Two variants of pump arrangements for arrangement 1000 are illustrated in FIG. 31A and in FIG. 31B respectively.

FIG. 31A illustrates a first embodiment of a pump arrangement Arr11A for the pBiVAD assist system or arrangement 1100, 1100A of FIG. 31 including an oxygenator device OXY11A.

Arrangement 1100A comprises:

a pump arrangement Arr11A, and

the oxygenator device OXY11A.

Pump arrangement Arr11A may comprise:

a membrane pump MP11Aa and a membrane pump MP11Ab,

a connecting portion CP11Ac which connects gas ports of membrane pump MP11Aa and a membrane pump MP11Ab with an IABP console IAPB11A.

A single liquid flow port of membrane pump MP11Aa is connected to a separate portion SP11Ac. A connecting portion CP11Aa is between separated portion SP11Ac and two further separated portions SP11Aa, SP11Ab. Separated portion SP11Aa is connected with the proximal portion PP1100b of cannula CA1100b. Separated portion SP11Ab is connected with a connecting portion CP11Ad.

Connecting portion CP11Ad is furthermore fluidically connected to an input port of oxygenator device OXY11A via a portion P11A1 as well as to a separate portion SP11Ad.

A single liquid flow port (RP) of membrane pump MP11Ab is connected to a separate portion SP11Af. A connecting portion CP11Ab is between separated portion SP11Af and two further separated portions SP11Ad, SP11Ae. Separated portion SP11Ad is connected with connecting portion CP11Ad as already mentioned. Separated portion SP11Ae is connected with proximal portion PP1100a of cannula CA1100a.

The output port of oxygenator device OXY11A is connected to a portion P11A2. Portion P11A2 is fluidically connected to proximal portion PP1100c of cannula CA1100c.

There may be the following one-way valves within arrangement 1100A:

one-way valve V11Aa within separated portion SP11Aa,

one-way valve V11Ab within separated portion SP11Ab,

one-way valve V11Ac within separated portion SP11Ad, and

one-way valve V11Ad within separated portion SP11Ae.

Arrangement 1100A comprises the oxygenator device OXY11A which enhances oxygen in the liquid B. Arrangement 1100A is or may be configured to be connected to the oxygenator OXY11A device. Arrangement 1100A is or may be configured such that the outflow of both membrane pump devices MP11Aa, MP11Ab or of other pump devices flows through oxygenator device OXY11A. Arrangement 1100A comprises preferably the cannula system CS and is configured to be connected to dual lumen cannula system DL-CS1100 which comprises at least inner cannula CA1100a which is arranged inside of outer cannula CA1100b and to single lumen cannula CA1100c. Arrangement 1100A may be modified if compared with FIG. 31A and may still have the same function.

The function of arrangement 1100A may be as follows:

an arrow A11Aa illustrates inflow from outer cannula CA1100a (RA) through separated portion SP11Ae,

an arrow A11Ab illustrates flow from membrane pump MP11Ab to oxygenator device OXY11A via connecting portions CP11Ab and CP11Ad,

an arrow A11Ac illustrates inflow from inner cannula CA1100b (LA) through separated portion SP11Aa,

an arrow A11Ad illustrates flow from membrane pump MP11Aa to oxygenator device OXY11A via connecting portions CP11Aa, CP11Ad, and

an arrow A11Ae illustrates flow from output port of oxygenator device OXY11A to single lumen cannula CA1100c.

Thus, all blood B1, B2 which flows into arrangement 1100A is oxygenated and a pulsatile flow is provided in outflow of arrangement 1100A, see arrows A11Ae and A1100e.

FIG. 31B illustrates a second embodiment of a pump arrangement Arr11B for the pBiVAD® assist system or arrangement 1000, 1100B of FIG. 31 including an oxygenator device OXY11B and allowing pulsatile outflow.

Other parts of the arrangement 1100B which is illustrated in FIG. 31B correspond to parts mentioned in FIG. 31A, e.g.:

separated portions SP12Ba to SP12Bf to separated portions SP11Aa to SP11Af respectively,

connecting portions CP12Ba to C12Bc to connecting portions CP11Aa to CP11Ac,

oxygenator device OXY12B to oxygenator device OXY11A,

one-way valves V12Ba to V12Bd to one-way valves V11Aa to V11Ad respectively, and

IABP console IAPB12B to IABP11A.

There may be the following differences:

no connecting portion which corresponds to connecting portion CP11Ad,

a connecting portion CP11Be downstream of oxygenator device OXY11B,

separated portion SP11Bb bypasses oxygenator device OXY11B and is directly connected to connecting portion CP11Be,

optional one-way valves V11Be within separated portion SP11Bb, e.g. there may be two one-way valves V11Bb and V11Be within this separated portion, and/or V11Bf, within portion P11B2,

a portion P11B2 from an output port of oxygenator device to connecting portion CP11Be, and

a portion P11B3 from connecting portion CP11Be to cannula CA1100c.

Arrangement 1100B comprises oxygenator device OXY11B which enhances oxygen in liquid B. Arrangement 1100B is or may be configured to be connected to oxygenator OXY11B device. Arrangement 1100B is or may be configured such that the outflow of one pump device, for instance of membrane pump device MP11Bb, of the at least two pump devices, for instance membrane pump devices MP11Ba, MP11Bb flows through oxygenator device OXY11A but not the outflow of the other pump device, for instance membrane pump MP11Ba, of the at least two pump devices, for instance of membrane pump devices MP11Ba, MP11Bb. Arrangement 1100B may comprise cannula system CS and may be configured to be connected to dual lumen cannula system (DL-CS1100) which comprises at least one inner cannula CA1100a which is arranged inside of an outer cannula CA1100b and to a single lumen cannula CA1100c. Arrangement 1100B may be modified if compared with FIG. 31B and may still have the same function.

The function of arrangement 1100B is as follows:

an arrow A11Ba illustrates inflow from outer cannula CA1100a (RA) through separated portion SP11Be,

an arrow A11Bb illustrates flow from membrane pump MP11Bb to oxygenator device OXY11B via connecting portion CP11Bb,

an arrow A11Bc illustrates inflow from inner cannula CA1100b (LA) through separated portion SP11Ba,

an arrow A11Bf illustrates flow from membrane pump MP11Ba to connecting portion CP11Be bypassing oxygenator device OXY11B,

an arrow A11Bg illustrates flow from output port of oxygenator device OXY11A to connecting portion CP11Be, and

an arrow A11Bh illustrates flow from connecting portion CP11Be to single lumen cannula CA1100c.

Thus, only blood B1 which flows into arrangement 1100B from right atrium RA is oxygenated. Blood B2 from left atrium LA which is already oxygenated by lung L is not oxygenated within arrangement 1100B. Pulsatile flow is provided in outflow of arrangement 1100B, see arrows A11Bh and A1100e.

In all embodiments one of the following methods may be used to bring or guide a guide wire and/or a catheter around or along the acute angle within the left ventricle LV. At least one snare may be used to catch the catheter and/or the guide wire in the left ventricle LV. The methods may be performed independent whether there is jugular access or a femoral access or another access for the catheter and/or the guide wire.

Variant A (catching the catheter with the snare):

1) Introducing a catheter through the right atrium RA, the atrial septum AS (a puncturing step may be performed earlier or using the catheter, e.g. using a needle and/or RF (radio frequency) tip/wire within the catheter). The catheter may be introduced further through the hole (puncture) in the atrial septum AS through left atrium LA, through mitral valve MV into the left ventricle LV.

2) Introducing a snare from descending aorta AO through aortic valve AV into left ventricle LV. This step may be performed also before step 1.

3) Catching the catheter in the left ventricle LV using the snare.

4) Pulling the snare and the distal end of the catheter therewith to the aorta AO.

5) Introducing a guide wire through the catheter.

6) Forwarding the guide wire out of the distal end of the catheter. Slight loosening of the snare may be optionally performed thereby.

7) As the guide wire is already within the snare, pull back the snare to a region in which only the guide wire is located but not the catheter.

8) Fix the guide wire using the snare, e.g. contract the snare and/or tighten the snare.

9) Optional, externalizing for instance the distal end of the guide wire out of the body. This step is optionally, because the proximal end of the snare is already outside of the body.

10) Remove catheter, e.g. pull back the catheter.

11) Introduce cannula using the guide wire, e.g. pushing the cannula along and/or over the guide wire until it is on its final place.

Variant B (catching the guide wire with the snare):

1) Introducing a catheter through the right atrium RA, through the atrial septum AS (a puncturing step may be performed earlier or through catheter, use needle and/or RF (radio frequency) tip/wire). Introducing the catheter further through left atrium LA, mitral valve MV into the left ventricle LV.

2) Introducing a guide wire through the catheter until the distal end of the guide wire comes out of the distal end of the catheter within the left ventricle LV. The RF wire may be used also as a guide wire.

3) Introducing a snare from descending aorta AO through aortic valve AV into left ventricle LV. This step may be performed before step 1 and/or before step 2.

3) Catching the distal end of the guide wire in the left ventricle LV using the snare.

4) Fixation of the guide wire using the snare.

5) Pulling the snare and the distal end of the guide wire therewith to the aorta AO.

6) Optional, externalizing guide wire by pulling it out of the body using the snare. This step is optional as the snare is already outside of the body from where it has been introduced.

7) Remove catheter, e.g. by pulling it back along the guide wire.

8) Introduce cannula over/along the guide wire until it is on place.

The following method may also be used in all corresponding embodiments for introducing a cannula jugularly transseptally:

1) Introduce a first snare into an internal jugular vein IJV, for instance into the right jugular vein RJV or into the left jugular vein LJV.

2) Advancing the first snare to inferior vena cava IVC.

3) Introducing a catheter into a common femoral vein CFV (left or right).

4) Advancing the catheter through the first snare into an inferior vena cava IVC.

5) Advancing the catheter through the first snare into the vena cava VC in an antegrade fashion.

6) Advancing the catheter through the first snare into the right atrium RA in an antegrade fashion.

7) Advancing the catheter through the first snare and from the right atrium RA transseptally through the atrial septum into the left atrium LA in an antegrade fashion. Puncturing of atrial septum may have been performed earlier. Alternatively, the catheter is used to puncture the atrial septum, for instance using a needle or using a RF (radio frequency) wire/tip which is introduced trough the catheter.

8) Advancing the catheter through the first snare and advancing the catheter across the mitral valve MV and into the left ventricular outflow tract, e.g. the left ventricle LV.

9) Advancing a second snare in the ascending aorta AO catching and snaring a distal portion of the catheter (Variant A) within the left ventricle LV. The second snare may optionally be introduced through an artery, which may include, but is not limited to, a radial artery, a brachial artery, an axillary artery, a subclavian artery, a carotid artery, or common femoral artery, and advanced retrograde into the aorta AO and into the left ventricle LV. The second snare may be already introduced before the catheter is introduced. Alternatively, a guide wire may be inserted into the catheter until a distal end of the guide wire comes out of a distal opening of the catheter. This distal end of the guide wire is then caught and snared within the left ventricle (Variant B)

10) Pulling the catheter (Variant A) or the guide wire (Variant B) into the aorta AO in an antegrade fashion using the second snare.

11) In variant A, advancing a guide wire through the catheter and through the first snare in antegrade fashion to the ascending aorta AO and through the second snare. Snaring the distal end of the guide wire in variant A but not the catheter.

12) In both variants A and B remove the catheter with the guide wire remaining in the heart H and through the first snare after the catheter is removed.

13) Externalizing a proximal portion of the guide wire from femoral vein, through inferior vena cava IVC, through inferior vena cava SVC, into the internal jugular vein IJV and then out of the internal jugular vein IJV using the first snare, for instance left jugular vein LJV or right jugular vein RJV. In some embodiments the snare may externalize a different portion of the guide wire, for instance an intermediate portion.

14) Advancing a cannula using the guide wire and/or along and/or over the guide wire from the internal jugular vein IJV. The cannula may be any of the cannulas described in this specification or known in the art. Especially, an outer cannula may be advanced over the guide wire from the internal jugular vein IJV. An inner cannula may optionally be advanced through a port proximal of the distal end of the outer cannula. The inner cannula and the outer cannula may be positioned as described in this description, or if a single multi-lumen cannula is used, it may be positioned in a similar manner.

15) Optionally, a distal portion of the guide wire may be externalized out of the body through the artery. This step is optional because the second snare is already externalized and may form a secure anchor for the distal portion of the guide wire.

Subclavian arteries/veins or other arteries/veins may be used for introducing the snare(s) because the snares require smaller diameters, e.g. less than 10 French (1 French equal to ⅓ mm (millimeter)) or less than 8 French, e.g. more than 3 French, compared to the diameters of the cannula(s).

In the following details of a method for puncturing transseptally through the atrial septum AS of the heart H are provided. However, other methods may be used as well, for instance using a needle. A catheter and/or a wire may be used which has a distal tip which can be heated, for instance using RF (radio frequency) energy, alternating current (ac), direct current (dc) etc. Thus, e.g. a hole may be burned into the septum, e.g. the atrial septum AS, during puncturing, for instance using temperatures above 100° C. (degrees Celsius) or above 200° C., less than 1000° C. for instance.

The RF (radio frequency) may be in the range of 100 kHz (kilohertz) to 1 MHz (Megahertz) or in the range of 300 kHz to 600 kHz, for instance around 500 MHz, i.e. in the range of 450 kHz to 550 kHz, e.g. 468 kHz.

The power of the radio frequency energy may have a maximum of 50 Watt. A power range of 5 W (watt) to 100 W may be used, for instance a range of 10 W to 50 W.

A sinus current/voltage may be used for the RF. The sinus current/voltage may be continuous. Alternatively, a pulsed sinus current/voltage may be used for the RF.

All parameters or some of the parameters of the RF equipment may be adjustable by an operator who performs the puncturing, for instance dependent on the specifics of the septum, e.g. normal septum, fibrotic septum, aneurysmal septum, etc. Preferably, the power may be adjustable.

A solution of Baylis Medical (may be a trademark), Montreal, Canada may be used, for instance NRG® trans-septal needle or Supra Cross® RF Wire technology. RF generator of type RFP-100A or a further development of this model may be used. This RF generator uses for example a frequency of 468 kHz (kilohertz).

A single puncture of the septum may be performed from a jugular access or from a femoral access or from another appropriate access using the RF energy. Smaller angles may be possible for the catheter if for instance compared with a needle.

Alternatively, the RF method may be used also if two separate punctures are made in the septum. However, usage of needles is possible as well. One of the punctures using the RF method may be made through left jugular vein LJV and the other puncture of the atrial septum AS may be made through the right jugular vein RJV.

It is possible to introduce both guide wires first through the atrial septum AS. Preferably, separate holes are used for each of the guide wires. Guide wire(s) may be used which include an RF tip. Alternatively, the wire(s) having the RF tip may be pulled back and a further wire may be introduced through the catheter.

Only after both guide wires are in place, both cannulas may be introduced using a respective one of the guide wires.

Alternatively, the first puncture may be performed using RF energy or a needle. Thereafter, the first cannula for blood transfer is inserted using the first guide wire. After insertion of the first cannula, the second puncture may be made. A second guide wire or the first guide wire may be used to introduce the second cannula.

Puncturing of the atrial septum may be assisted by at least one medical imaging method, preferably by at least two medical imaging methods.

US (ultra-sonic) echo imaging may be used to visualize the movement of heart H and the location of the valves of heart H. No dangerous radiation may result from ultra-sonic imaging. An ultra-sonic transmitter may be introduced for instance via the esophagus, e.g. trans esophagus echo (TEE) may be used.

X-ray radiation preferably in combination with fluorescence (fluoroscopy), may be used in order to visualize the location of catheters (comprising for instance at least one X-ray marker, or the devises are usually radiopaque) and/or the location of guide wire(s), snares etc.

Thus, transseptal puncturing or puncturing of other tissue may be guided by TEE and by fluoroscopy or by other imaging methods. At least two different image generating methods may be used.

In all embodiments mentioned above, it is also possible to use a soft guide wire and a stiffer guide wire which does not bend so easy if compared with the soft guide wire. The following steps may be performed, preferably in combination with snaring:

1) Introduce a soft guide wire.

2) Introduce catheter using the soft wire as a guide.

3) Optionally, remove soft wire, for instance by pulling back the soft wire out of the catheter.

4) Introduce stiffer guide wire into the catheter, e.g. there may be a change of wire from soft wire to the stiffer wire.

The catheter may be removed, e.g. pulled back. Thereafter, the stiffer wire may be used to introduce a cannula or cannulas.

The RF puncturing or another thermal puncturing may be used to puncture a hole from coronary sinus vein through wall of left atrium. Thus, the arc that is formed by the coronary sinus vein does not form a hindrance during puncturing because the thermal tip, for instance the RF tip may be guided to the wall and is thereafter activated in order to burn the hole which is later used to introduce the cannula from the coronary sinus CS into the left atrium LA. At least two different image generating methods may be used to puncture the wall of the left atrium from the coronary sinus vein CSV, e.g. the puncturing may be guided by TEE and by fluoroscopy or by other imaging methods.

Thus, RF puncturing may be used at least once for puncturing through the coronary sinus vein CSV. The atrial septum AS or another septum of the heart H may also be punctured using RF energy or other thermal methods. However, the atrial septum AS may also be punctured using a needle, for instance if a femoral access is used.

Although embodiments of the present disclosure and their advantages have been described in detail, it should be understood that various changes, substitutions and alterations can be made therein without departing from the spirit and scope of the disclosure as defined by the appended claims. For example, it will be readily understood by those skilled in the art that many of the features, functions, processes and methods described herein may be varied while remaining within the scope of the present disclosure. Moreover, the scope of the present application is not intended to be limited to the particular embodiments of the system, process, manufacture, method or steps described in the present disclosure. As one of ordinary skill in the art will readily appreciate from the disclosure of the present disclosure systems, processes, manufacture, methods or steps presently existing or to be developed later that perform substantially the same function or achieve substantially the same result as the corresponding embodiments described herein may be utilized according to the present disclosure. Accordingly, the appended claims are intended to include within their scope such systems, processes, methods or steps. Further, it is possible to combine embodiments mentioned in the first part of the description with examples of the second part of the description which relates to FIGS. 1 to 31B. Moreover, it is possible to combine embodiments mentioned in the first part of the Figures, i.e. FIGS. 1 to 11, with examples of the second part of the Figures, which relates to FIGS. 12 to 31B.

1. Cannula system (CS1 to CS3, 210, 310, 1010, 1040, 1110) comprising:
a first cannula (O1 to O3) having a minimum inner diameter or width,
a second cannula (I1 to I3) having a maximum outer diameter or width, wherein the minimum inner diameter or width is greater than the maximum outer diameter or width,
wherein the second cannula (I1 to I3) is adapted to be guided into the first cannula (O1 to O3) such that a first lumen of the first cannula (O1 to O3) remains in the first cannula (O1 to O3) between an outer surface the second cannula (I1 to I3) and an inner surface of the first cannula (O1 to O3),
wherein the first lumen of the first cannula (O1 to O3) defines a first fluid conduit and a first lumen of the second cannula (I1 to I3) defines a second fluid conduit.

2. Cannula system (CS1 to CS3, 210, 310, 1010, 1040, 1110) according to claim 1, wherein the first lumen of the first cannula (O1 to O3) is limited by an outer surface of the second cannula (I1 to I3) such that fluid guidable in the first lumen of the first cannula (O1 to O3) is in physical contact with the outer surface of the second cannula (I1 to I3).

3. Cannula system (CS1 to CS3, 210, 310, 1010, 1040, 1110) according to one of the preceding claims, wherein the first cannula (O1 to O3) is flexible and bendable such that it is insertable into a vessel of the body, preferably a blood vessel,
and wherein the first cannula (O1 to O3) is flexible and bendable such that it is guidable into the internal jugular vein (IJV), the subclavian artery or the subclavian vein, thereafter into the superior vena cava (SVC) and then into the right atrium (RA) at least up to or through the atrial septum, or intravascular to the superior vena cava (SVC) and up to the right atrium (RA) or up to the right ventricle (RV).

4. Cannula system (CS1 to CS3, 210, 310, 1010, 1040, 1110) according to one of the preceding claims, wherein a portion of the first cannula (O1 to O3) through which the second cannula (I1 to I3) is guidable within the first cannula (O1 to O3) has a length of at least 20 cm or at least 40 cm, and wherein the second cannula (I1 to I3) is guidable beyond a distal end of the first cannula (O1 to O3) by at least 5 cm or by at least 10 cm,
and/or wherein the first cannula (O1 to O3) and/or the second cannula (I1 to I3) is pre-bended or both cannulas are pre-bended by an angle within the range of 60 degrees to 175 degrees or within the range of 70 to 145 degrees, preferably in order to ease an insertion through the septum of the heart, preferably through the atrial septum.

5. Cannula system (CS1 to CS3, 210, 310, 1010, 1040, 1110) according to one of the preceding claims, wherein the first cannula (O1 to O3) comprises a first coupling port (P1a, P1b), wherein the first coupling port (P1a, P1b) is adapted to be coupled or is coupled to a blood pump, and/or wherein the second cannula (I1 to I3) comprises a second coupling port (P2a, P2b), wherein the second coupling port (P2a, P2b) is adapted to be coupled or is coupled to a blood pump or to the blood pump.

6. Cannula system (CS1 to CS3, 210, 310, 1010, 1040, 1110) according to one of the preceding claims, comprising an opening (OP1, OP2) in the outer wall of the first cannula (O1 to O3) through which the second cannula (I1 to I3) is insertable into the lumen of the first cannula (O1 to O3), wherein the opening (OP2) is located within a sidewall of the first cannula (O2) or wherein the opening (OP1) is located at a proximal end of the first cannula (O1), preferably centrally with respect to a longitudinal axis (A) of the first cannula (O1 to O3),
and wherein the cannula system comprises at least one sealing element (S1, S2) that is arranged between the first cannula (O1 to O3) and the second cannula (I1 to I3) preferably arranged within the opening (OP1, OP2), and preferably comprising a retaining ring, a sealing ring, a gasket, a multi-flap valve or another self-sealing member.

7. Cannula system (CS1 to CS3, 210, 310, 1010, 1040, 1110) according to one of the preceding claims, comprising a closure element that prevents the passage of fluid through the distal end of the outer cannula beyond the closure element into the first lumen of the first cannula (O1 to O3), preferably a multi-flap valve or another self-sealing member,
and wherein preferably the closure element is adapted to be in a closed state or in an at least partially open state if the second cannula (I1 to I3) and/or an introducer (2114) is arranged within an opening of the closure element.

8. Cannula system (CS1 to CS3, 210, 310, 1010, 1040, 1110) according to one of the preceding claims, comprising a first introducer for introducing at least the first cannula (O1 to O3) and a second introducer for introducing the second cannula (I1 to I3),
and wherein the second introducer is longer than the first introducer and wherein the second introducer is thinner than the first introducer.

9. Cannula system (CS1 to CS3, 210, 310, 1010, 1040, 1110) according to one of the preceding claims, wherein the first cannula (O1) is configured such that the second cannula (I1) is arranged coaxially within the first cannula (O1) if inserted into the first cannula (O1), preferably along the whole length of an overlapping region of the first cannula (O1) and of the second cannula (I1) or along at least 90 percent of the length of the overlapping region.

10. Cannula system (CS1 to CS3, 210, 310, 1010, 1040, 1110) according to one of the preceding claims, comprising a first diameter variable arrangement mounted on the first cannula (O1 to O3), preferably a first cage arrangement (1086), and wherein the first diameter variable arrangement has an expanded state having a maximum outer diameter that is greater than the maximum outer diameter in a non-expanded state of the diameter variable arrangement, preferably greater by at least factor two or at least factor three,
and/or comprising a second diameter variable arrangement mounted on the second cannula (I1 to I3), preferably a second cage arrangement (1046),
and wherein the second diameter variable arrangement has an expanded state having a maximum outer diameter that is greater than the maximum outer diameter in a non-expanded state of the second diameter variable arrangement, preferably greater by at least factor two or by at least factor three.

11. Cannula system (CS1 to CS3, 210, 310, 1010, 1040, 1110) according to claim 10, wherein the first cage arrangement (1086) and/or the second cage arrangement (1046) comprises a plurality of cage wires (1048, 1088),
and wherein the first cage arrangement (1086) and/or the second cage arrangement (1046) comprises a proximal portion (1631), wherein in the expanded state of the cage arrangement the distance between neighboring wires (1048, 1088) in the proximal portion increases with increasing distance to a mounting portion of the wires (1048, 1088),
and wherein the first cage arrangement (1086) and/or wherein the second cage arrangement (1046) comprises a distal portion (1633), wherein in the expanded state of the cage arrangement the distance between neighboring wires (1048, 1088) in the distal portion decreases with increasing distance to the mounting portion of the wires (1048, 1088),
and wherein preferably the first cage arrangement (1086) and/or the second cage arrangement (1046) comprise following the distal portion a backwardly bended portion (1834), wherein preferably at least one or all of the plurality of wires is backward bended by more than 90 degrees, by more than 110 degrees, by more than 120 degrees, or more than 140 degrees,
and wherein preferably there is this a radial portion, having preferably a radial length in the range of 3 mm to 10 mm (millimeters), in which at least one or all of the plurality of wires extend only radially, and wherein preferably the first cage arrangement (1086) and/or the second cage arrangement (1046) comprises an optional transition portion (1632), wherein in the expanded state of the cage arrangement the distance between neighboring wires (1048, 1088) in the optional transition portion is constant with increasing distance to the mounting portion of the wires (1048, 1088).

12. Cannula system (CS1 to CS3, 210, 310, 1010, 1040, 1110) according to claim 11, wherein the proximal ends of the cage wires (1088) of the first cage arrangement (1096) on the first cannula (O1 to O3) are wound around an outer surface of the first cannula (O1 to O3),
and/or wherein proximal ends of the cage wires (1048) of the second cage arrangement (1046) on the second cannula (I1 to I3) are wound around an outer surface of the second cannula (I1 to I3), and wherein the distal ends of the cage wires (1048, 1088) are connected with each other, preferably using a connecting element and/or by twisting them together.

13. Cannula system (CS1 to CS3, 210, 310, 1010, 1040, 1110) according to one of the claims 11 to 12, wherein in the expanded state the cage wires (1048, 1088) are distributed angularly such that, in a given axial position, for two different pairs of neighboring cage wires the wires (1048, 1088) forming the pair have respectively a first distance relative to each other which is equal for both pairs and that neighboring wires (1048, 1088) forming yet another pair of neighboring wires have a second distance relative to each other that is greater than the first distance, preferably equal to or greater than twice the first distance.

14. Cannula system (CS1 to CS3, 210, 310, 1010, 1040, 1110) according to one of the claims 10 to 13, wherein the first diameter variable arrangement comprises a first membrane (1089), wherein the first membrane (1089) is folded or less stretched in the non-expanded state of the first diameter variable arrangement and wherein the first membrane (1089) is expanded in the expanded state of the first diameter variable arrangement,
and/or wherein the second diameter variable arrangement comprises a second membrane (1049), wherein the second membrane (1049) is folded or less stretched in the non-expanded state of the second diameter variable arrangement and wherein the second membrane (1049) is expanded in the expanded state of the second diameter variable arrangement.

15. Cannula system (CS1 to CS3, 210, 310, 1010, 1040, 1110) according to claim 14, wherein in the expanded state of the first diameter variable arrangement an edge of the first membrane defines an opening that faces distally relative to a longitudinal axis of the first cannula (O1 to O3),
and/or wherein in the expanded state of the second diameter variable arrangement an edge of the second membrane (1049) defines an opening that faces distally relative to the longitudinal axis of the second cannula (I1 to I3).

16. Cannula system (CS1 to CS3, 210, 310, 1010, 1040, 1110) according to claim 14, wherein in the expanded state of the first diameter variable arrangement an edge of the first membrane (1089) defines an opening that faces laterally relative to a longitudinal axis of the first cannula (O1 to O3),
and/or wherein in the expanded state of the second diameter variable arrangement an edge of the second membrane defines an opening that faces laterally relative to the longitudinal axis of the second cannula (I1 to I3).

17. Cannula system (CS1 to CS3, 210, 310, 1010, 1040, 1110) according to one of the preceding claims, comprising at least one fixation element (FE1, FE2) that is configured to prevent an axial movement of the second cannula (I1 to I3) relative to the first cannula (O1 to O3) after complete insertion of the second cannula (I1 to I3), wherein preferably the fixation element (FE1, FE2) is arranged on an outside of the first cannula (O1 to O3) and/or on an outside of the second cannula (I1 to I3).

18. Cannula system (210, 310) according to one of the preceding claims, wherein the first cannula (O1 to O3) is adapted to be inserted intravascular, preferably jugular, through the superior vena cava (SVC) into an interior region of the heart (H) and wherein the second cannula (I1 to I3) is adapted to be inserted through the first cannula (O1 to O3) into a different interior region of the heart (H) compared to the interior region where the first cannula (O1 to O3) is positioned, or into the aorta (AO),
and wherein preferably at least the second cannula (I1 to I3) is adapted to be inserted further into the left atrium (LA), the left ventricle (LV) and the aorta (AO), preferably into the ascending aorta (aAO), and wherein a diameter variable arrangement is mounted on the distal end portion (122) of the second cannula (I1 to I3).

19. Cannula system (1040) according to one of the claims 1 to 17, wherein the first cannula (O1 to O3) is adapted to be inserted intravascular through the vena cava, preferably through the superior vena cava (SVC), into the left atrium (LA),
and wherein the second cannula (I1 to I3) is adapted to be inserted through the first cannula (O1 to O3), through the left atrium (LA), through the left ventricle (LV) to the aorta (AO), preferably to the ascending aorta (aAO),
wherein a first diameter variable arrangement is mounted on the distal end portion of the first cannula (O1 to O3) and is covered with a first membrane (1089), wherein the first membrane (1089) in an expanded state of the first diameter variable arrangement preferably has an opening facing laterally relative to the first cannula (O1 to O3) and/or an edge that is essentially parallel to two cage wires (1088) of the first diameter variable arrangement,
and wherein a second diameter variable arrangement is mounted on the distal end portion (1042) of the second cannula (I1 to I3) and is covered with a second membrane (1049), wherein the second membrane (1049) in an expanded state of the second diameter variable arrangement preferably has an opening facing distally relative to the second cannula (I1 to I3) and/or an edge that is essentially transversally to cage wires (1048) of the second diameter variable arrangement.

20. Cannula system (1010, 1110) according to one of the claims 1 to 17, wherein the first cannula (O1 to O3) is adapted to be inserted intravascular into an interior region of the heart (H) and wherein the second cannula (I1 to I3) is adapted to be inserted intravascular through the heart (H) into the pulmonary artery (PA), into the left pulmonary artery (WA), into the right pulmonary artery (rPA) and/or into the lung (L),
wherein preferably the first cannula (O1 to O3) is adapted to be inserted intravascular through the vena cava, preferably through the superior vena cava (SVC), up to the right atrium (RA) or up to the right ventricle (RV),
and wherein preferably a first diameter variable arrangement is mounted on the distal end portion of the first cannula (O1 to O1),
and wherein preferably a second diameter variable arrangement is mounted on the distal end portion (1042) of the second cannula (I1 to I3), preferably covered with a membrane (1049), wherein the membrane (1049) in an expanded state of the diameter variable arrangement preferably has an opening facing distally relative to the second cannula (I1 to I3) and/or an edge that is essentially transversally to cage wires (1048) of the diameter variable arrangement.

21. Assembly of a cannula system (CS1 to CS3, 210, 310, 1010, 1040, 1110) according to one of the preceding claims and of at least one blood pump, preferably a membrane pump (MP), wherein the at least on blood pump is fluidly connected to the first cannula (O1 to O3) and to the second cannula (I1 to I3).

22. Method for cannulizing a subject, comprising:
inserting a first cannula (O1 to O3) into a body (100) of the subject,
after insertion of the first cannula (O1 to O3), guiding a second cannula (I1 to I3) through the first cannula (O1 to O3) into the body (100) of the subject,
whereby a first lumen of the first cannula (O1 to O3) is left outside of the second cannula (I1 to I3), and thereafter guiding a first fluid through the first lumen of the first cannula (O1 to O3) and guiding the first fluid or a second fluid through a first lumen of the second cannula (I1 to I3).

23. Method according to claim 22,
wherein the first cannula (O1 to O3) is inserted along a vessel of the body (100), preferably along a blood vessel,
wherein preferably the first cannula (O1 to O3) is inserted into the body (100) along a length of at least 20 cm or of at least 30 cm, especially through the right jugular vein,
wherein preferably the first cannula (O1 to O3) is inserted into the body (100) along a length of at least 35 cm or of at least 40 cm of at least 45 cm of at least 50 cm of at least 55 cm of at least 60 cm of at least 65 cm of at least 70 cm, especially through the left jugular vein,
and wherein the second cannula (I1 to I3) is inserted into the body (100) along a length that is at least 10 cm or at least 15 cm or at least 20 cm or at least 25 cm or at least 30 cm longer than the length along which the first cannula (O1 to O3) is inserted into the body (100),
and/or wherein the first cannula (O1 to O3) is bended by an angle within the range of 60 degrees to 175 degrees or within the range of 70 degrees to 145 degrees.

24. Method according to one of the claims 22 to 23, wherein the first fluid is guided into a first direction within the first lumen of the first cannula (O1 to O3) and wherein the first fluid or the second fluid is guided into the same or into an opposite direction compared to the first direction within the first lumen of the second cannula (I1 to I3),
and/or wherein the first fluid and/or the second fluid is the same fluid, preferably blood or a fluid comprising blood or components of blood.

25. Method according to one of the claims 22 to 24, wherein the second cannula (I1) is arranged coaxially within the first cannula (O1), preferably along the whole length of the first cannula (O1) or preferably at least along at least 90 percent of the length of the first cannula (O1),
and wherein preferably the first cannula (O1) has a circular or oval cross section having an outer diameter or a maximal outer diameter in the range of 7 mm or 8 mm to 10 mm or 11 mm (Millimeter), i.e. 21 F or 24 F (French) to 31 or 33 F (French), and wherein preferably the second cannula (I1) has a circular or oval cross section having an outer diameter or a maximal outer diameter that is at least 2 mm or at least 3 mm or at least 4 mm smaller than an inner diameter or a maximal inner diameter of the first cannula (O1).

26. Method according to one of the claims 22 to 24, wherein the second cannula (I2, I3) is arranged outside a central position of the first cannula (O2, O3), preferably along the whole overlapping length of the first cannula (O2, O3) and the second cannula (I2, I3) if the second cannula (I2, I3) is inserted completely into the first cannula (O2, O3) or preferably at least along at least 90 percent of the overlapping length,
and wherein preferably the first cannula (I2, I1) has a circular or oval cross section having an outer diameter or a maximal outer diameter in the range of 7 mm or 8 mm to 10 mm or 11 mm (Millimeter), i.e. 21 F or 24 F (French) to 31 F or 33 F (French).

27. Method according to one of the claims 22 to 26, wherein the second cannula (I1 to I3) is fixed against an axial movement relative to the first cannula after complete insertion of the second cannula, wherein a fixation element (FE1, FE2) is used, preferably outside of first cannula (O1 to O3) and/or of the body (100).

28. Method according to one of the claims 22 to 27, wherein the first cannula (O1 to O3) is inserted intravascular, preferably jugular, preferably through superior vena cava (SVC), into an interior region of the heart (H) and wherein the second cannula (I1 to I3) is inserted through the first cannula (O1 to O3) into an interior region of the heart (H), preferably to a different interior region of the heart (H) compared to the interior region where the first cannula (O1 to O3) is positioned, or into the aorta (AO).

29. Method according to claim 28, wherein a diameter variable arrangement is mounted on the distal end portion of the first cannula (O1 to O3),
and wherein the first cannula does not extend into the diameter variable arrangement or does maximally extend into the diameter variable arrangement by at most 10 mm or at most 5 mm or at most 3 mm.

30. Method according to claim 29, wherein only the diameter variable arrangement is arranged in the left atrium (LA) and the distal end portion of the first cannula (O1 to O3) is arranged within the right atrium (RA).

31. Method according to one of the claims 28 to 30, wherein the first cannula (O1 to O3) and/or the second cannula (I1 to I3) is punctured through the atrial septum of the heart (H) and wherein the second cannula (I1 to I3) is inserted further into the left atrium (LA), the left ventricle (LV) and the aorta (AO), preferably into the ascending aorta (aAO),
and wherein a diameter variable arrangement is mounted on the distal end portion (122) of the second cannula (I1 to I3).

32. Method according to one of the claims 22 to 27, wherein the first cannula (O1 to O3) is inserted intravascular into an interior region of the heart (H) and wherein the second cannula (I1 to I2) is inserted intravascular into the pulmonary artery (PA), into the left pulmonary artery (WA), into the right pulmonary artery (rPA) and/or into the lung (L).

33. Method according to claim 32, wherein the first cannula (O1 to O3) is inserted intravascular through the vena cava, preferably through the superior vena cava (SVC) up to the right atrium (RA) or up to the right ventricle (RV),
and wherein a diameter variable arrangement is mounted on the distal end portion of the second cannula (I1 to I3), preferably covered with a membrane (1049, 1149) that has preferably an opening that faces distally and or that has preferably an edge that is essentially transversally to cage wires (1048, 1148) of the diameter variable arrangement.

34. Method according to claim 33 wherein the first cannula (O1 to O3) comprises holes that are placed within the right atrium (RA) and within the right ventricle (RV), especially drainage holes.

35. Method according to one of the claims 22 to 34, wherein there is a first diameter variable arrangement mounted on a distal end portion of the first cannula (O1 to O3), preferably a first cage arrangement (1086) and wherein the first diameter variable arrangement has an expanded state having a maximum outer diameter that is greater than the maximum outer diameter in a stretched state, preferably at least by factor two or at least by factor three,
and/or wherein there is a second diameter variable arrangement mounted on a distal end portion of the second cannula (I1 to I3), preferably a second cage arrangement (1046), and wherein the second diameter variable arrangement has an expanded state having a maximum outer diameter that is greater than the maximum outer diameter in a stretched state, preferably at least by factor two or at least by factor three.

36. Method according to claim 35, wherein the first cage arrangement (1086) and/or the second cage arrangement (1046) comprises a plurality of wires (1048, 1088),
and/or wherein the first cage arrangement (1086) and/or the second cage arrangement (1046) comprises a proximal portion (1631), wherein within the proximal portion in the expanded state of the cage arrangement the distance between neighboring wires (1048, 1088) increases with increasing distance to a mounting portion of the wires (1048, 1088),
and wherein the first cage arrangement (1086) and/or the second cage arrangement (1046) comprises a distal portion (1633), wherein within the distal portion in the expanded state of the cage arrangement the distance between neighboring wires (1048, 1088) decreases with increasing distance to the mounting portion of the wires (1048, 1088),
and wherein preferably the first cage arrangement (1086) and/or the second cage arrangement (1046) comprises an optional transition portion (1632), wherein within the optional transition portion in the expanded state of the cage arrangement the distance between neighboring wires (1048, 1088) is constant with increasing distance to the mounting portion of the wires (1048, 1088).

37. Method according to claim 36 wherein proximal ends of the cage wires (1088) of the first cage arrangement (1086) on the first cannula (O1 to O3) are wound around an outer surface of the first cannula (O1 to O3) and/or around the distal end portion of the first cannula (O1 to O3),
and/or wherein proximal ends of the cage wires (1048) of the second cage arrangement (1046) on the second cannula (II to 13) are wound around an outer surface of the second cannula (II to 13) and/or around the distal end portion (1042) of the second cannula (I1 to I3),
and wherein the distal ends of the cage wires (1048, 1088) are connected with each other, preferably using a connecting element and/or by twisting them with each other.

38. Method according to claim 36 or 37, wherein the first cage arrangement (1086) and/or the second cage arrangement (1046) comprise following the distal portion a backwards bended portion (1834).

39. Method according to one of the claims 36 to 38, wherein the first diameter variable arrangement comprises a first membrane (1089), wherein the first membrane (1089) is folded or less stretched in the non-expanded state of the first diameter variable arrangement and wherein the first membrane (1089) is spanned in the expanded state of the first diameter variable arrangement,
and/or wherein the second diameter variable arrangement comprises a second membrane (1049), wherein the second membrane (1049) is folded or less stretched in the non-expanded state of the second diameter variable arrangement and wherein the second membrane (1049) is spanned in the expanded state of the second diameter variable arrangement,
and wherein at least one of the membranes (1049) comprises an opening facing distally and/or wherein at least one of the membranes (1089) comprises an opening facing laterally.

40. Method according to one of the claims 22 to 39, wherein the first cannula (O1 to O3) and the second cannula (I1 to I2) form a first multi lumen cannula system (CS1) and wherein a second multi lumen cannula system is used at the same time within the same body (100), preferably inserted in the same organ as the first multi lumen cannula system (CS1).

41. Method according to claim 40, wherein a first cannula of the second multi lumen cannula system is inserted into the body (100),
wherein after insertion of the first cannula of the second multi lumen cannula system a second cannula of the second multi lumen cannula system is inserted axially within the first cannula of the second multi lumen cannula system, preferably until it extends axially beyond the first cannula of the second multi lumen cannula.

42. Method according to claim 40 or 41,
wherein the distal end of the first cannula (O1 to O3) of the first multi lumen cannula system (CS1) is placed within left atrium (LA),
wherein the distal end of the second cannula of the first multi lumen cannula system (CS1) is placed within the aorta (AO), preferably within the ascending aorta (aAO),
wherein a first diameter variable arrangement is mounted on the distal end portion of the first cannula (O1 to O3) of the first multi lumen cannula system (CS1), preferably covered with a membrane (1089) that has an opening that faces laterally and/or that is essentially parallel to two of the cage wires (1088) of the diameter variable arrangement,
and wherein a second diameter variable arrangement is mounted on the distal end portion (1042) of the second cannula (I1 to I3) of the first multi lumen cannula system (CS1), preferably covered with a membrane (1049) that preferably has an opening facing distally and/or that is essentially transversally to the cage wires of the diameter variable arrangement,
wherein the distal end of the first cannula of the second multi lumen cannula system is placed within right atrium (RA) or the right ventricle (RV), and
wherein the distal end of the second cannula of the second multi lumen cannula system is placed within the pulmonary artery (PA), preferably within the left pulmonary artery (lPA) or within the right pulmonary artery (rPA),
and wherein a third diameter variable arrangement is mounted on the distal end portion of the second cannula of the second multi lumen cannula system, preferably covered with a membrane that has preferably an opening facing distally and/or that is essentially transversally to the cage wires of the diameter variable arrangement.

43. Method according to claim 42, wherein the following flows are established:
a first flow from a distal end of the second cannula of the second multi lumen cannula system through the second cannula of second multi lumen cannula out of an outlet port of the second cannula of the second multi lumen cannula system through a first circuitry, preferably comprising a pump (P10a), into an inlet port of first cannula of the first multi lumen cannula system (CS1) through the first cannula (O1 to O3) of the first multi lumen cannula system (CS1) out of distal end of the first cannula (O1 to O3) of the first multi lumen cannula system (CS1),
a second flow from a distal end of the first cannula of the second multi lumen cannula system through the first cannula of the second multi lumen cannula system through a first circuitry, preferably comprising a group of a pump (P10b) and an oxygenator device (OXY10), into an inlet port of the second cannula of the first multi lumen cannula system (CS1) through the second cannula (I1 to I3) of the first multi lumen cannula system (CS1) out of the distal end of the second cannula (I1 to I3) of the first multi lumen cannula system (CS1).

44. Method according to claim 42, wherein the following flows are established:
a first flow from a distal end of first cannula (O1 to O3) of first multi lumen cannula system (CS1) through first cannula (O1 to O3) of first multi lumen cannula system (CS1) out of an outlet port of first cannula (O1 to O3) of first multi lumen cannula system (CS1) through an first circuitry, preferably comprising a pump (P10a), into an inlet port of the second cannula of second multi lumen cannula system through the second cannula of second multi lumen cannula system out of distal end of the second cannula of the second multi lumen cannula system,
a second flow from a distal end of the first cannula of the second multi lumen cannula system through the first cannula of the second multi lumen cannula system through a first circuitry, preferably comprising a group of a pump (P10b) and an oxygenator device (OXY10), into an inlet port of the second cannula (I1 to I3) of the first multi lumen cannula system (CS1) through the second cannula (I1 to I3) of the first multi lumen cannula system (CS1) out of the distal end of the second cannula (I1 to I3) of the first multi lumen cannula system (CS1).

45. Method according to one of the claims 22 to 44, wherein at least one sealing element (S1, S2) is arranged within an opening (OP1, OP2) through which the second cannula (I1 to I3) is inserted into the first cannula (O1 to O3),
preferably a retaining ring, a sealing ring, a gasket, a multi-flap valve or another self-sealing element.

46. Method according to one of the claims 22 to 45, wherein there is a closure element that prevents the passage of fluid through the distal end of the first cannula (O1 to O3) into the first lumen of the first cannula (O1 to O3) and/or vice versa, wherein the closure element is arranged preferably on a distal end portion of the first cannula (O1 to O3), preferably a closure element that allows the passage of the second cannula (I1 to I3) and/or of a introducer,
wherein the closure element comprises at least one membrane, preferably at least one membrane comprising an aperture and/or at last one self-sealing element, e.g. a multi-flap valve.

47. Method according to one of the claims 21 to 46, wherein the first fluid and/or the second fluid is injected into the body (100) and/or taken out of the body (100) in a pulsed fluid flow or in a continuous fluid flow.

48. Method according to one of the claims 22 to 47, wherein the first cannula (O1 to O3) and/or the second cannula (I1 to I3) is pre-bended or both cannulas are pre bended by an angle within the range of 60 degrees to 175 degrees or in the range of 70 degrees to 145 degrees, preferably in order to ease an insertion through the septum of the heart, preferably through the atrial septum or the ventricle septum.

49. Method according to any one of the claims 22 to 39,
wherein the distal end of the first cannula (O1 to O3) is placed within left atrium (LA),
wherein the distal end of the second cannula is placed within the aorta (AO), preferably within the ascending aorta (aAO),
wherein preferably a first diameter variable arrangement is mounted on the distal end portion of the first cannula (O1 to O3), preferably covered with a membrane (1089) that has an opening that faces laterally and/or that is essentially parallel to two of the cage wires (1088) of the diameter variable arrangement,
and wherein preferably a second diameter variable arrangement is mounted on the distal end portion (1042) of the second cannula (I1 to I3), preferably covered with a membrane (1049) that preferably has an opening facing distally and/or that is essentially transversally to the cage wires of the diameter variable arrangement,
wherein a further cannula (CA29a) is inserted into the coronary sinus vein (CSV) and through a puncture between the coronary sinus vein (CSV) and the left atrium (LA) up to the left atrium (LA).

50. Method according to any one of the claims 22 to 39, wherein a biventricular assist arrangement (1100) is realized which is configured to drain blood (B) from the left atrium (LA) through at least one opening (DO1100a) of the second cannula (CA1100a) and to drain blood from the right atrium (RA) through at least one opening (OP1100) of the first cannula (CA1100b),
wherein preferably the second cannula (CA1100a) is inserted into the first cannula (CA1100b) within the body.

51. Method according to one of the claims 22 to 50, wherein a cannula system (CS1 to CS3, 210, 310, 1010, 1040, 1110, DL-CS700, DLCS-1100) according to one of the claims 1 to 20 or an assembly according to claim 21 is used.
